SEQUENCE LISTING (28 sequences)

SEQ ID NO: 1 - RNA, 21 nt, Homo sapiens
auuuauuuauuuauuuauuua

SEQ ID NO: 2 - RNA, 17 nt, Homo sapiens
auuuauuuauuuauuua

SEQ ID NO: 3 - RNA, 15 nt, Homo sapiens
wauuuauuuauuuaw

SEQ ID NO: 4 - RNA, 13 nt, Homo sapiens
wwauuuauuuaww

SEQ ID NO: 5 - RNA, 13 nt, Homo sapiens
wwwwauuuawwww

SEQ ID NO: 6 - DNA, 1643 bp, Homo sapiens
gcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaattgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgctt

SEQ ID NO: 7 - DNA, 756 bp, Homo sapiens
gctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttct

SEQ ID NO: 8 - DNA, 756 bp, Homo sapiens
tctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttct

SEQ ID NO: 9 - DNA, 825 bp, Homo sapiens
atcactctctttaatcactactcacattaacctcaactcctgccacaatgtacaggatgcaactcctgtcttgcattgcactaattcttgcacttgtcacaaacagtgcacctacttcaagttcgacaaagaaaacaaagaaaacacagctacaactggagcatttactgctggatttacagatgattttgaatggaattaataattacaagaatcccaaactcaccaggatgctcacatttaagttttacatgcccaagaaggccacagaactgaaacagcttcagtgtctagaagaagaactcaaacctctggaggaagtgctgaatttagctcaaagcaaaaactttcacttaagacccagggacttaatcagcaatatcaacgtaatagttctggaactaaagggatctgaaacaacattcatgtgtgaatatgcagatgagacagcaaccattgtagaatttctgaacagatggattaccttttgtcaaagcatcatctcaacactaacttgataattaagtgcttcccacttaaaacatatcaggccttctatttatttatttaaatatttaaattttatatttattgttgaatgtatggttgctacctattgtaactattattcttaatcttaaaactataaatatggatcttttatgattctttttgtaagccctaggggctctaaaatggtttaccttatttatcccaaaaatatttattattatgttgaatgttaaatatagtatctatgtagattggttagtaaaactatttaataaatttgataaatataaaaaaaaaaaacaaaaaaaaaaa

SEQ ID NO: 10 - RNA, 15 nt, Homo sapiens
  misc_feature (1)..(1): n is a, c, g, or u
nauuuauuuauuuan

SEQ ID NO: 11 - DNA, 1125 bp, Homo sapiens
ttctgccctcgagcccaccgggaacgaaagagaagctctatctcgcctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattc

SEQ ID NO: 12 - DNA, 3166 bp, Homo sapiens
aagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagttaccacctcctccccggccggcggcggacagtggacgcggcggcgagccgcgggcaggggccggagcccgcccccggaggcggggtggagggggtcggagctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagcgcgcgcgctccccagccctggcccggcctcgggccgggaggaagagtagctcgccgaggcgccgaggagagcgggccgccccacagcccgagccggagagggacgcgagccgcgcgccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctccaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacacactcgcgttgcaaggcgaggcagcttgagttaaacgaacgtacttgcagatgtgacaagccgaggcggtgagccgggcaggaggaaggagcctccctcagggtttcgggaaccagatctctctccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaagaatgtgcaaggccagggcatgggggcaaatatgacccagttttgggaacaccgacaaacccagccctggcgctgagcctctctaccccaggtcagacggacagaaagacaaatcacaggttccgggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggaggccactggcagatgtcccggcgaagagaagagacacattgttggaagaagcagcccatgacagcgccccttcctgggactcgccctcatcctcttcctgctccccttcctggggtgcagcctaaaaggacctatgtcctcacaccattgaaaccactagttctgtccccccaggaaacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattagaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaagagggtttcaatatacatctacatactatatatatattgggcaacttgtatttgtgtgtatatatatatatatatgtttatgtatatatgtgatcctgaaaaaataaacatcgctattctgttttttatatgttcaaaccaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaa

SEQ ID NO: 13 - RNA, 249 nt, Homo sapiens
ccgggcucauggacgggugaggcggcggugugcgcagacagugcuccagcgcgcgcgcuccccagcccuggcccggccucgggccgggaggaagaguagcucgccgaggcgccgaggagagcgggccgccccacagcccgagccggagagggacgcgagccgcgcgccccggucgggccuccgaaaccaugaacuuucugcugucuugggugcauuggagccuugccuugcugcucuaccuccaccaug

SEQ ID NO: 14 - DNA, 9181 bp, Homo sapiens
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 15 - RNA, 29 nt, Homo sapiens
ggcagaucugagccugggagcucucugcc

SEQ ID NO: 16 - RNA, 52 nt, Homo sapiens
uuuuuuagggaagaucuggccuuccuacaagggaaggccagggaauuuucuu

SEQ ID NO: 17 - DNA, 9413 bp, Homo sapiens
ttgggggcgacactccaccatagatcactcccctgtgaggaactactgtcttcacgcagaaagcgtctagccatggcgttagtatgagtgttgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgttaagttcccgggcggtggtcagatcgttggtggagtttacctgttgccgcgcaggggccccaggttgggtgtgcgcgcgactaggaagacttccgagcggtcgcaacctcgtggaaggcgacaacctatccccaaggctcgccggcccgagggtaggacctgggctcagcccgggtacccttggcccctctatggcaacgagggtatggggtgggcaggatggctcctgtcaccccgtggctctcggcctagttggggccccacagacccccggcgtaggtcgcgtaatttgggtaaggtcatcgatacccttacatgcggcttcgccgacctcatggggtacattccgcttgtcggcgcccccctagggggcgctgccagggccctggcacatggtgtccgggttctggaggacggcgtgaactatgcaacagggaatctgcccggttgctctttctctatcttcctcttagctttgctgtcttgtttgaccatcccagcttccgcttacgaggtgcgcaacgtgtccgggatataccatgtcacgaacgactgctccaactcaagtattgtgtatgaggcagcggacatgatcatgcacacccccgggtgcgtgccctgcgtccgggagagtaatttctcccgttgctgggtagcgctcactcccacgctcgcggccaggaacagcagcatccccaccacgacaatacgacgccacgtcgatttgctcgttggggcggctgctctctgttccgctatgtacgttggggatctctgcggatccgtttttctcgtctcccagctgttcaccttctcacctcgccggtatgagacggtacaagattgcaattgctcaatctatcccggccacgtatcaggtcaccgcatggcttgggatatgatgatgaactggtcacctacaacggccctagtggtatcgcagctactccggatcccacaagccgtcgtggacatggtggcgggggcccactggggtgtcctagcgggccttgcctactattccatggtggggaactgggctaaggtcttgattgtgatgctactctttgctggcgttgacgggcacacccacgtgacagggggaagggtagcctccagcacccagagcctcgtgtcctggctctcacaaggcccatctcagaaaatccaactcgtgaacaccaacggcagctggcacatcaacaggaccgctctgaattgcaatgactccctccaaactgggttcattgctgcgctgttctacgcacacaggttcaacgcgtccgggtgcccagagcgcatggctagctgccgccccatcgatgagttcgctcaggggtggggtcccatcactcatgatatgcctgagagctcggaccagaggccatattgctggcactacgcgcctcgaccgtgcgggatcgtgcctgcgtcgcaggtgtgtggtccagtgtattgcttcactccgagccctgttgtagtggggacgaccgatcgtttcggcgctcctacgtatagctggggggagaatgagacagacgtgctgctacttagcaacacgcggccgcctcaaggcaactggtttgggtgcacgtggatgaacagcactgggttcaccaagacgtgcgggggccctccgtgcaacatcgggggggtcggcaacaacaccttggtctgccccacggattgcttccggaagcaccccgaggccacttacacaaagtgtggctcggggccctggttgacacccaggtgcatggttgactacccatacaggctctggcactacccctgcactgttaactttaccgtctttaaggtcaggatgtatgtggggggcgtggagcacaggctcaatgctgcatgcaattggactcgaggagagcgctgtgacttggaggacagggataggtcagaactcagcccgctgctgctgtctacaacagagtggcagatactgccctgttccttcaccaccctaccggccctgtccactggcttgatccatcttcaccggaacatcgtggacgtgcaatacctgtacggtatagggtcggcagttgtctcctttgcaatcaaatgggagtatatcctgttgcttttccttcttctggcggacgcgcgcgtctgtgcctgcttgtggatgatgctgctgatagcccaggctgaggccaccttagagaacctggtggtcctcaatgcggcgtctgtggccggagcgcatggccttctctccttcctcgtgttcttctgcgccgcctggtacatcaaaggcaggctggtccctggggcggcatatgctctctatggcgtatggccgttgctcctgctcttgctggccttaccaccacgagcttatgccatggaccgagagatggctgcatcgtgcggaggcgcggtttttgtaggtctggtactcttgaccttgtcaccatactataaggtgttcctcgctaggctcatatggtggttacaatattttatcaccagagccgaggcgcacttgcaagtgtgggtcccccctctcaatgttcggggaggccgcgatgccatcatcctccttacatgcgcggtccatccagagctaatctttgacatcaccaaactcctgctcgccatactcggtccgctcatggtgctccaggctggcataactagagtgccgtactttgtacgcgctcaggggctcatccgtgcatgcatgttagtgcggaaggtcgctggaggccactatgtccaaatggccttcatgaagctggccgcgctgacaggtacgtacgtatatgaccatcttactccactgcgggattgggcccacgcgggcctacgagaccttgcggtggcagtagagcccgtcgtcttctctgacatggagactaaactcatcacctggggggcagacaccgcggcgtgtggggacatcatctcgggtctaccagtctccgcccgaagggggaaggagatacttctaggaccggccgatagttttggagagcaggggtggcggctccttgcgcctatcacggcctattcccaacaaacgcggggcctgcttggctgtatcatcactagcctcacaggtcgggacaagaaccaggtcgatggggaggttcaggtgctctccaccgcaacgcaatctttcctggcgacctgcgtcaatggcgtgtgttggaccgtctaccatggtgccggctcgaagaccctggccggcccgaagggtccaatcacccaaatgtacaccaatgtagaccaggacctcgtcggctggccggcgccccccggggcgcgctccatgacaccgtgcacctgcggcagctcggacctttacttggtcacgaggcatgctgatgtcgttccggtgcgccggcggggcgacagcagggggagcctgctttcccccaggcccatctcctacctgaagggctcctcgggtggaccactgctttgcccttcggggcacgttgtaggcatcttccgggctgctgtgtgcacccggggggttgcgaaggcggtggacttcatacccgttgagtctatggaaactaccatgcggtctccggtcttcacagacaactcatcccctccggccgtaccgcaaacattccaagtggcacatttacacgctcccactggcagcggcaagagcaccaaagtgccggctgcatatgcagcccaagggtacaaggtgctcgtcctaaacccgtccgttgccgccacattgggctttggagcgtatatgtccaaggcacatggcatcgagcctaacatcagaactggggtaaggaccatcaccacgggcggccccatcacgtactccacctattgcaagttccttgccgacggtggatgctccgggggcgcctatgacatcataatatgtgatgaatgccactcaactgactcgactaccatcttgggcatcggcacagtcctggatcaggcagagacggctggagcgcggctcgtcgtgctcgccaccgccacgcctccgggatcgatcaccgtgccacaccccaacatcgaggaagtggccctgtccaacactggagagattcccttctatggcaaagccatccccattgaggccatcaaggggggaaggcatctcatcttctgccattccaagaagaagtgtgacgagctcgccgcaaagctgacaggcctcggactcaatgctgtagcgtattaccggggtctcgatgtgtccgtcataccgactagcggagacgtcgttgtcgtggcaacagacgctctaatgacgggttttaccggcgactttgactcagtgatcgactgcaacacatgtgtcacccagacagtcgatttcagcttggatcccaccttcaccattgagacgacaacgctgccccaagacgcggtgtcgcgtgcgcagcggcgaggtaggactggcaggggcaggagtggcatctacaggtttgtgactccaggagaacggccctcaggcatgttcgactcctcggtcctgtgtgagtgctatgacgcaggctgcgcttggtatgagctcacgcccgctgagacctcggttaggttgcgggcttacctaaatacaccagggttgcccgtctgccaggaccacctagagttctgggagagcgtcttcacaggcctcacccacatagatgcccacttcttgtcccagaccaaacaggcaggagacaacctcccctacctggtagcataccaagccacagtgtgcgccagggctcaggctccacctccatcgtgggaccaaatgtggaagtgtctcatacggctaaagcccacactgcatgggccaacgcccctgctgtacaggctaggagccgttcaaaatgaggtcactctcacacaccccataaccaaatacatcatggcatgcatgtcggctgacctggaggtcgtcactagcacctgggtgctagtaggcggagtccttgcggctctggccgcgtactgcctgacgacaggcagcgtggtcattgtgggcaggatcatcttgtccgggaggccagctgttattcccgacagggaagtcctctaccaggagttcgatgagatggaagagtgtgcttcacacctcccttacatcgagcaaggaatgcagctcgccgagcaattcaaacagaaggcgctcggattgctgcaaacagccaccaagcaagcggaggctgctgctcccgtggtggagtccaagtggcgagcccttgaggtcttctgggcgaaacacatgtggaacttcatcagcgggatacagtacttggcaggcctatccactctgcctggaaaccccgcgatagcatcattgatggcttttacagcctctatcaccagcccgctcaccacccaaaataccctcctgtttaacatcttggggggatgggtggctgcccaactcgctccccccagcgctgcttcggctttcgtgggcgccggcattgccggtgcggccgttggcagcataggtctcgggaaggtacttgtggacattctggcgggctatggggcgggggtggctggcgcactcgtggcctttaaggtcatgagcggcgagatgccctccactgaggatctggttaatttactccctgccatcctttctcctggcgccctggttgtcggggtcgtgtgcgcagcaatactgcgtcggcacgtgggcccgggagagggggctgtgcagtggatgaaccggctgatagcgttcgcttcgcggggtaaccacgtctcccccacgcactatgtgcccgagagcgacgccgcggcgcgtgttactcagatcctctccagccttaccatcactcagttgctgaagaggcttcatcagtggattaatgaggactgctccacgccttgttccggctcgtggctaaaggatgtttgggactggatatgcacggtgttgagtgacttcaagacttggctccagtccaagctcctgccgcggttaccgggactccctttcctgtcatgccaacgcgggtacaagggagtctggcggggggatggcatcatgcaaaccacctgcccatgtggagcacagatcaccggacatgtcaaaaatggctccatgaggattgttgggccaaaaacctgcagcaacacgtggcatggaacattccccatcaacgcatacaccacgggcccctgcacgccctccccagcgccgaactattccagggcgctgtggcgggtggctgctgaggagtacgtggaggttacgcgggtgggggatttccactacgtgacgggcatgaccactgacaacgtgaaatgcccatgccaggttccagcccctgaatttttcacggaggtggatggagtacggttgcacaggtatgctccagtgtgcaaacctctcctacgagaggaggtcgtattccaggtcgggctcaaccagtacctggtcgggtcacagctcccatgtgagcccgaaccggatgtggcagtgctcacttccatgctcaccgacccctctcatattacagcagagacggccaagcgtaggctggccagggggtctcccccctccttggccagctcttcagctagccagttgtctgcgccttctttgaaggcgacatgtactacccatcatgactccccggacgctgacctcatcgaggccaacctcctgtggcggcaggagatgggcgggaacatcacccgtgtggagtcagaaaataaggtggtaatcctggactctttcgatccgattcgggcggtggaggatgagagggaaatatccgtcccggcggagatcctgcgaaaacccaggaagttccccccagcgttgcccatatgggcacgcccggattacaaccctccactgctagagtcctggaaggacccggactacgtccccccggtggtacacgggtgccctttgccatctaccaaggcccccccaataccacctccacggaggaagaggacggttgtcctgacagagtccaccgtgtcttctgccttggcggagctcgctactaagacctttggcagctccgggtcgtcggccgttgacagcggcacggcgactggccctcccgatcaggcctccgacgacggcgacaaaggatccgacgttgagtcgtactcctccatgccccccctcgagggagagccaggggaccccgacctcagcgacgggtcttggtctaccgtgagcggggaagctggtgaggacgtcgtctgctgctcaatgtcctatacatggacaggtgccttgatcacgccatgcgctgcggaggagagcaagttgcccatcaatccgttgagcaactctttgctgcgtcaccacagtatggtctactccacaacatctcgcagcgcaagtctgcggcagaagaaggtcacctttgacagactgcaagtcctggacgaccactaccgggacgtgctcaaggagatgaaggcgaaggcgtccacagttaaggctaggcttctatctatagaggaggcctgcaaactgaagcccccacattcggccaaatccaaatttggctacggggcgaaggacgtccggagcctatccagcagggccgtcaaccacatccgctccgtgtgggaggacttgctggaagacactgaaacaccaattgataccaccatcatggcaaaaaatgaggttttctgcgtccaaccagagaaaggaggccgcaagccagctcgccttatcgtattcccagacctgggggtacgtgtatgcgagaagatggccctttacgacgtggtctccacccttcctcaggccgtgatgggcccctcatacggattccagtactctcctgggcagcgggtcgagttcctggtgaatacctggaaatcaaagaaatgccctatgggcttctcatatgacacccgctgctttgactcaacggtcactgagaatgacatccgtactgaggaatcaatttaccaatgttgtgacttggcccccgaagccaggcaggccataaggtcgctcacagagcggctttatgtcgggggtcccctgactaattcgaaggggcagaactgcggttatcgccggtgccgcgcaagtggcgtgctgacgactagctgcggcaacaccctcacatgttacttgaaggccactgcggcctgtcgagctgcaaagctccaggactgcacgatgctcgtgaacggagacgaccttgtcgttatctgtgagagtgcgggaacccaggaggatgcggcggccctacgagccttcacggaggctatgactaggtattccgccccccccggggacccgccccaaccagaatacgacttggagctgataacgtcatgctcctccaatgtgtcggtcgcgcacgatgcatccggcaaaagggtgtactacctcacccgtgaccccaccacccccctcgcacgggctgcgtgggagacagttagacacactccagtcaactcctggctaggcaatatcatcatgtatgcgcccaccctatgggcgaggatgattctgatgactcatttcttctctatccttctagctcaggagcaacttgaaaaagccctggattgtcagatctacggggcctgttactccattgagccacttgacctacctcagatcattgaacgactccatggtcttagcgcattttcactccacagttactctccaggtgagatcaatagggtggcttcatgcctcaggaaacttggggtaccgcctttgcgagtctggagacatcgggccagaagtgtccgcgctaagctactgtcccagggggggagggctgccacttgcggcaagtacctcttcaactgggcagtaaagaccaagcttaaactcactccaatcccggctgcgtcccagctagacttgtccggctggttcgttgctggttacaacgggggagacatatatcacagcctgtctcgtgcccgaccccgttggttcatgttgtgcctactcctactttctgtaggggtaggcatctacctgctccccaaccggtgaacggggagctaaccactccaggccaataggccattcccttttttttttttc

SEQ ID NO: 18 - RNA, 328 nt, Homo sapiens
uugggggcgacacuccaccauagaucacuccccugugaggaacuacugucuucacgcagaaagcgucuagccauggcguuaguaugaguguugugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaguaguguugggucgcgaaaggccuugugguacugccugauagggugcuugcgagugccccgggaggucucguagaccgugcau

SEQ ID NO: 19 - RNA, 14 nt, Homo sapiens
auuugggcgugccc

SEQ ID NO: 20 - RNA, 27 nt, Homo sapiens
gccgaguaguguugggucgcgaaaggc

SEQ ID NO: 21 - DNA, 340 bp, Homo sapiens
atgggcggagggaagctcatcagtggggccacgagctgagtgcgtcctgtcactccactcccatgtcccttgggaaggtctgagactagggccagaggcggccctaacagggctctccctgagcttcagggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagaccactctcctccgcccatt

SEQ ID NO: 22 - DNA, 349 bp, Homo sapiens
gaggaaagtccgggctcacacagtctgagatgattgtagtgttcgtgcttgatgaaacaataaatcaaggcattaatttgacggcaatgaaatatcctaagtctttcgatatggatagagtaatttgaaagtgccacagtgacgtagcttttatagaaatataaaaggtggaacgcggtaaacccctcgagtgagcaatccaaatttggtaggagcacttgtttaacggaattcaacgtataaacgagacacacttcgcgaaatgaagtggtgtagacagatggttatcacctgagtaccagtgtgactagtgcacgtgatgagtacgatggaacagaacgcggcttat

SEQ ID NO: 23 - DNA, 377 bp, Homo sapiens
gaagctgaccagacagtcgccgcttcgtcgtcgtcctcttcgggggagacgggcggaggggaggaaagtccgggctccatagggcagggtgccaggtaacgcctgggggggaaacccacgaccagtgcaacagagagcaaaccgccgatggcccgcgcaagcgggatcaggtaagggtgaaagggtgcggtaagagcgcaccgcgcggctggtaacagtccgtggcacggtaaactccacccggagcaaggccaaataggggttcataaggtacggcccgtactgaacccgggtaggctgcttgagccagtgagcgattgctggcctagatgaatgactgtccacgacagaacccggcttatcggtcagtttcacct

SEQ ID NO: 24 - DNA, 38110 bp, Homo sapiens
ccaccggttacgatcttgccgaccatggccccacaatagggccggggagacccggcgtcagtggtgggcggcacggtcagtaacgtctgcgcaacacggggttgactgacgggcaatatcggctccatagcgtcggccgcggatacagtaaaggagcattctgtgacggaaaagacgcccgacgacgtcttcaaacttgccaaggacgagaaggtcgaatatgtcgacgtccggttctgtgacctgcctggcatcatgcagcacttcacgattccggcttcggcctttgacaagagcgtgtttgacgacggcttggcctttgacggctcgtcgattcgcgggttccagtcgatccacgaatccgacatgttgcttcttcccgatcccgagacggcgcgcatcgacccgttccgcgcggccaagacgctgaatatcaacttctttgtgcacgacccgttcaccctggagccgtactcccgcgacccgcgcaacatcgcccgcaaggccgagaactacctgatcagcactggcatcgccgacaccgcatacttcggcgccgaggccgagttctacattttcgattcggtgagcttcgactcgcgcgccaacggctccttctacgaggtggacgccatctcggggtggtggaacaccggcgcggcgaccgaggccgacggcagtcccaaccggggctacaaggtccgccacaagggcgggtatttcccagtggcccccaacgaccaatacgtcgacctgcgcgacaagatgctgaccaacctgatcaactccggcttcatcctggagaagggccaccacgaggtgggcagcggcggacaggccgagatcaactaccagttcaattcgctgctgcacgccgccgacgacatgcagttgtacaagtacatcatcaagaacaccgcctggcagaacggcaaaacggtcacgttcatgcccaagccgctgttcggcgacaacgggtccggcatgcactgtcatcagtcgctgtggaaggacggggccccgctgatgtacgacgagacgggttatgccggtctgtcggacacggcccgtcattacatcggcggcctgttacaccacgcgccgtcgctgctggccttcaccaacccgacggtgaactcctacaagcggctggttcccggttacgaggccccgatcaacctggtctatagccagcgcaaccggtcggcatgcgtgcgcatcccgatcaccggcagcaacccgaaggccaagcggctggagttccgaagccccgactcgtcgggcaacccgtatctggcgttctcggccatgctgatggcaggcctggacggtatcaagaacaagatcgagccgcaggcgcccgtcgacaaggatctctacgagctgccgccggaagaggccgcgagtatcccgcagactccgacccagctgtcagatgtgatcgaccgtctcgaggccgaccacgaatacctcaccgaaggaggggtgttcacaaacgacctgatcgagacgtggatcagtttcaagcgcgaaaacgagatcgagccggtcaacatccggccgcatccctacgaattcgcgctgtactacgacgtttaaggactcttcgcagtccgggtgtagagggagcggcgtgtcgttgccagggcgggcgtcgaggtttttcgatgggtgacggtggccggcaacggcgcgccgaccaccgctgcgaagagcccgtttaagaacgttcaaggacgtttcagccgggtgccacaacccgcttggcaatcatctcccgaccgccgagcgggttgtctttcacatgcgccgaaactcaagccacgtcgtcgcccaggcgtgtcgtcgcggccggttcaggttaagtgtcggggattcgtcgtgcgggcgggcgtccacgctgaccaacggggcagtcaactcccgaacactttgcgcactaccgcctttgcccgccgcgtcacccgtaggtagttgtccaggaattccccaccgtcgtcgtttcgccagccggccgcgaccgcgaccgcattgagctggcgcccgggtcccggcagctggtcggtgggcttgccgcgcaccaacaccagcgcgttgcgggcccgggtggcggtcagccaggcctgacggagcagctccacgtcggctgcgggaaccagatcggcggccgcgatgacatccagggattgcagcgtcgaggtgttgtgcagggcgggaacctggtgcgcatgctgtagctgcagcaactgcacggtccattcgatgtcggccagtccgccgcggcccagtttggtgtgtgtgttggggtcggcaccgcgcggcaaccgctcggactcgatacgggccttgatgcggcgaatctcgcgcaccgagtcagcggacacaccgtcgggcggataccgcgttttgtcgaccatccgtaggaatcgctgacccaactcggcatcgccggcaaccgcgtgtgcgcgtagcagggcctggatctcccatggctgtgcccactgctcgtagtatgcggcgtaggaccccagggtgcggaccagcggaccgttgcggccctcgggtcgcaaattggcgtcgagctccagcggcggatcgacgctgggtgtccccagcagcgcccgaacccgctcggcgatcgatgtcgaccatttcaccgcccgtgcatcgtcgacgccggtggccggctcacagacgaacatcacgtcggcatccgacccgtagcccaactcggcaccacccagccgacccatgccgatgaccgcgatggccgccggggcgcgatcgtcgtcgggaaggctggcccggatcatgacgtccagcgcggcctgcagcaccgccacccacaccgacgtcaacgcccggcacacctcggtgacctcgagcaggccgagcaggtccgccgaaccgatgcgggccagctctcgacgacgcagcgtgcgcgcgccggcgatggcccgctccgggtcggggtagcggctcgccgaggcgatcagcgcccgagccacggcggcgggctcggtctcgagcagcttcgggcccgcaggcccgtcctcgtactgctggatgacccgcggcgcgcgcatcaacagatccggcacatacgccgaggtacccaagacatgcatgagccgcttggccaccgcgggcttgtcccgcagcgtggccaggtaccagctttcggtggccagcgcctcactgagccgccggtaggccagcagtccgccgtcgggatcgggggcatacgacatccagtccagcagcctgggcagcagcaccgactgcacccgtccgcgccggccgctttgattgaccaacgccgacatgtgtttcaacgcggtctgcggtccctcgtagcccagcgcggccagccggcgccccgcggcctccaacgtcatgccgtgggcgatctccaacccggtcgggccgatcgattccagcagcggttgatagaagagtttggtgtgtaacttcgacacccgcacgttctgcttcttgagttcctcccgcagcaccccggccgcatcgtttcggccatcgggccggatgtgggccgcgcgcgccagccagcgcactgcctcctcgtcttcgggatcgggaagcaggtgggtgcgcttgagccgctgcaactgcagtcggtgctcgagcagcctgaggaactcatacgacgcggtcatgttcgccgcgtcctcacgcccgatgtagccgccttcgcccaacgccgccaatgcgtccaccgtggacgccacccgtaacgactcgtcgctacgggcatgaaccagctgcagtagctgtacggcgaactccacgtcgcgcaatccgccgctgccgagtttgagctcgcggccgcggacatcggcgggcaccagctgctccacccgccgccgcatggcctgcacctcgaccacaaagtcttcgcgctcgcaggctcgccacaccatcggcatcaaggcggtcaggtaacgctcgccaagttccgcgtcgccaacgactggccgtgctttcagcaacgcctgaaactcccaggtcttggcccagcgctggtagtaggcgatgtgcgactcgagcgtacggaccagctccccgttgcgcccctccggacgcagggcggcgtccacctcgaaaaaggccgccgaggccacccgcatcatctcgctggccacgcgcgcgttgcgcgggtcggagcgctcggcaacgaatatgacatcgacgtcgctgacgtagttcagttcgcgcgcaccgcacttgcccatcgcgatgaccgccaggcgcggtggcgggtgctcgccgcacacgctcgcctcggccacgcgcagcgccgccgccagagcggcgtccgcggcgtccgccaggcgtgcggccaccacggtgaatggcagcaccggttcgtcctcgaccgtcgcggccaggtcgagagcggccagcattagcacgtagtcgcggtactgggttcgcaatcggtgcacgagcgagcccggcataccctccgattcctcgacgcactcgacgaacgaccgctgcagctggtcatgggacggcagtgtgaccttgccccgcagcaatttccaggactgcggatgggcgaccaggtgatcgcccaacgccagcgacgagcccagcaccgagaacagccgcccgcgcagactgcgttcgcgcagcagagccgcgttgagctcgtcccatccggtgtctggattctccgacagccggatcaaggcgcgcagcgcggcatcggcgtccggagcgcgtgacagcgaccacagcaggtcgacgtgcgcctgatcctcgtgccgatcccaccccagctgagccagacgctcaccagcaggggggtcaactaatccgagccggccaacgctgggcaacttcggccgctgcgtggcgagtttggtcacgaccacgacggtagcgcaaagcgcgtcggcgtcggatcaaccggtagatctgggctacagcgacaggtaggtgcgcagctcgtatggcgtgacgtggctgcggtagttcgcccactccgtgcgcttgttgcgcaagaaaaagtcaaaaacgtgctcccccaaggcctccgcgacgagttcggaggcctccatggcgcgcagcgcactatccaaactggacggcaattctcggtaccccatcgctcggcgttcctcgggtgtgaggtcccatacgttgtcctcggcctgcgggcccagcacgtaacccttctctacaccccgcaatcccgcggccagcagcacggcgaatgtcagatagggattgcacgccgaatcagggctgcgtacttcgacccgccgcgacgaggtcttgtgcggcgtgtacatcggcacccgcactagggcggatcggttggcggccccccacgacgcggccgtgggcgcttcgccgccctgcaccagccgcttgtaagagttgacccactgatttgtgaccgcgctgatctcgcaagcgtgctccaggatcccggcgatgaacgatttacccacttccgacagctgcagcggatcatcagcgctgtggaacgcgttgacatcaccctcgaacaggctcatgtgggtgtgcatcgccgagcccgggtgctggccgaatggcttgggcatgaacgacgcccgggcgccctcttccagcgcgacttctttgatgacgtagcggaaggtcatcacgttgtcagccatcgacagagcgtcggcaaaccgcaggtcgatctcctgctggccgggtgcgccttcgtgatggctgaactccaccgagatgcccatgaattccagggcatcgatcgcgtggcggcgaaagttcaaggcggagtcgtgcaccgcttggtcgaaatagccggcgttgtcgaccgggacgggcaccgacccgtcctcgggtccgggcttgagcaggaagaactcgatttcgggatgcacgtagcaggagaagccgagttcgccggccttcgtcagctgccgccgcaacacgtgccgcgggtccgcccacgacggcgagccgtccggcatggtgatgtcgcaaaacatccgcgctgagtggtggtggccggaactggtggcccagggcagcacctggaaggtcgacgggtccgggtgcgccaccgtatcggattccgagacccgcgcaaagccctcgatcgaggatccgtcgaagccgatgccttcctcgaaggcgccctcgagttcggctggggcgatggcgaccgacttgaggaaaccgagcacgtctgtgaaccacagccggacgaagcggatgtcgcgttcttccagggtacgaagaacgaattccttctgtcggtccatacctcgaacagtatgcactgtctgttaaaaccgtgttaccgatgcccggccagaagcgttgcggggcggcccgcaaggggagtgcgcggtgagttcagggcgcgcaccgcagactcgtcggcggcaaggtcccgtcgagaaaatagtgcatcaccgcagagtccacacactggttgccatcgaacaccgcagtgtgttgggtgccgtcgaaggtgatcagcggtgcgcccagctggcgggccaggtctaccccggactgatacggagtggccgggtcgtgggtggtggacaccacgacgaccttgccagccccggccggcgccgcggggtgcggcgtcgacgttgccggcaccggccacagcgcgcacagatcgcggggggcggatccggtgaactgcccgtagctaaggaacggggcgacctgacggatccgttggtcggcggccacccaggccgctggatcggccggtgtgggcgcatcgacgcaccggaccgcgttgaacgcgtcctggtcgttgctgtagtgcccgtctgcatcccggccgtcatagtcgtcggcaagcaccagcaagtcgccggcgtcgctgccgcgctgcagccccagcagaccactggtcaggtacttccagcgctgagggctgtacagcgcgttgatggtgcccgtcgtcgcgtcggcgtagctcaggccacgtggatccgacgtcttacccggcttctgcaccagcgggtcaaccagggcgtggtagcggttgacccactgggccgagtcggtgcccagagggcaggccggcgagcgggcgcagtcggcggcgtagtcattgaaagcggtctgaaatcccgccatttggctgatgctttcctcgattgggctaacggctggatcgatagcgccgtcgaggaccatcgcccgcacatgagtaccgaaccgttccaggtaagcggtgcccaactcggtgccgtagctgtatccgaggtagttgatctgatcgtcacctaacgcttggcgaaccatgtccatgtcccgtgcgacggacgcggtaccgatattggccaagaagctgaagcccatccggtcaacacagtcctgggccaactgccggtagacctgttcgacgtgggtgacaccggccggactgtagtcggccatcggatcgcgccggtacgcgtcgaactcggcgtcggtgcgacaccgcaacgcaggggtcgagtggccgacccctctcgggtcgaagcccaccaggtcgaagtggcggagaatgtcggtgtcggcgatcgcgggtgccatagcggcgaccatgtcgaccgccgacgccccgggtcccccaggattgaccagcagtgctccgaatcgctgtcccgtcgcggggacgcggatcaccgccaacttcgcttgtgtcccaccgggttggtcgtagtcgacggggacggacaccgtcgcgcagcgtgcagtgcgaatttcgctggtgtcggcgatgaactcgcggcagctgttccaactctgttgcggcgccacgaccggcgcacccggggtttggccggcgccgggttcttcagtcgcgccggccaacgggggcgctgctaggggcagtccgccgagcagcaacccgaaggacagcagcgccgagctcaacggtctgcggcgccacatggccgccatcgtctcaccggcgaatacctgtgacggcgcgaaatgatcacaccttcgtttcttcgccccgctagcacttggcgccgctgggcggcgtggtgccgccgattaaatacgccgtcacgtactcgtcaatgcagctgtcgccctggaataccaccgtgtgctgggttccgtcgaaggtcagcaacgaaccgcgaagctggttcgccaggtcgaccccggccttgtacggcgtcgccgggtcatgggtggtggataccaccaccgtcggcactaggccgggcgccgagacggcatggggctgacttgtgggtggcaccggccagaacgcgcaggtgcccagcggcgcatcaccggtgaacttcccgtagctcatgaacggtgcgatctcccgggcgcggcggtcttcgtcgatgaccttgtcgcgatcggtaaccgggggctgatcgacgcaattgatcgccacccgcgcgtcaccggaattgttgtagcggccgtgcgagtcccgacgcatgtacatgtcggccagagccagcagggtgtctccgcgattgtcgaccagctccgacagcccgtcggtcaagtgttgccacagattcggtgagtacagcgccataatggtgcccacgatggcgtcgctataactcagcccgcgcggatccttcgtgcgcgccggcctgctgatcctcgggttgtccgggtcgaccaacggatcgaccaggctgtggtagacctcgacggctttggccgggtcggcgcccagcgggcagcccgcgttcttggcgcagtcggcggcatagttgttgaacgcgtcctggaagcccttggcctggcgcagctccgcctcgatgggatcggcattggggtcgacggcaccgtcgagaatcattgcccgcacccgctgcggaaattcctcggcatacgcggagccgatccgggtgccgtacgagtagcccaggtaggtcagcttgtcgtcgcccaacgccgcgcgaatggcatccaggtccttggcgacgttgaccgtcccgacatgggccagaaagttcttgcccatcttgtccacacagcgaccgacgaattgcttggtctcgttctcgatgtgcgccacaccctcccggctgtagtcaacctgcggctcggcccgcagccggtcgttgtcggcatcggagttgcaccagatcgccggccgggacgacgccaccccgcgggggtcgaacccaaccaggtcgaacctttcgtgcacccgcttcggcaatgtctggaagacgcccaaggcggcctcgataccggattcgccgggtccaccgggatttatgaccagcgaaccgatcttgtctcccgtcgccggaaagcgaatcagcgccagcgccgccacgtcaccatcggggcggtcgtagtcgaccggtacagcgagcttgccgcataacgcgccgccggggatctttacttgcgggtttgacgaccggcacggtgtccactccaccggctggcccagcttcggctccgccatacgagcgcgtcccccgaccacgcggatgcagcccacaagaaccaacgccacggcggcgagcgcggcccagatcaacagcatgcgcgcgatcttgtcgcggcgagacagcctcatgcccacaatgctgccagagcagacccgagatcctggccagcggccaccgtcggccgactaaccggccgctgccagcagtcctgccatcgccgatggcgaactcgtcggccatcccccatacgtccggtaacagatccgggcaagacaccgacccgtcgaccggatccggcacgggcgcgtcggcctcggcggtgcacaactgcgacatcaggttggcgctggcaccccgtccacgccggcatggtgcaccttggccatcgcccgagggcgatccccgatgccgtccaccccttcgacgaacccatctcccacggcggtcgccggcagcgacgcgatgtggccgcagatctccgagagttcggcccgcccgcccggcgacggcaacccgatgccgtgcaagtgacgatcgatgtgaggttcaaggttcagcgcactgctggcaagctttttccgaaaccgcggcctcgccttgatctggagtcagaacgcgtcacgcagccggtcaaaggcgtaacccatgctcgagcaaacatgcatgggctgagtggacgtttccagacacagcaactggcgtccaggccactgagccgctgcatgcgcgatggtatgccgatgggggccccgggcgcgtctgaggggaagaagtggcagactgtcagggtccgacgaacccggggaccctaacgggccacgaggatcgacccgaccaccattagggacagtgatgtctgagcagactatctatggggccaatacccccggaggctccgggccgcggaccaagatccgcacccaccacctacagagatggaaggccgacggccacaagtgggccatgctgacggcctacgactattcgacggcccggatcttcgacgaggccggcatcccggtgctgctggtcggtgattcggcggccaacgtcgtgtacggctacgacaccaccgtgccgatctccatcgacgagctgatcccgctggtccgtggcgtggtgcggggtgccccgcacgcactggtcgtcgccgacctgccgttcggcagctacgaggcggggcccaccgccgcgttggccgccgccacccggttcctcaaggacggcggcgcacatgcggtcaagctcgagggcggtgagcgggtggccgagcaaatcgcctgtctgaccgcggcgggcatcccggtgatggcacacatcggcttcaccccgcaaagcgtcaacaccttgggcggcttccgggtgcagggccgcggcgacgccgccgaacaaaccatcgccgacgcgatcgccgtcgccgaagccggagcgtttgccgtcgtgatggagatggtgcccgccgagttggccacccagatcaccggcaagcttaccattccgacggtcgggatcggcgctgggcccaactgcgacggccaggtcctggtatggcaggacatggccgggttcagcggcgccaagaccgcccgcttcgtcaaacggtatgccgatgtcggtggtgaactacgccgtgctgcaatgcaatacgcccaagaggtggccggcggggtattccccgctgacgaacacagtttctgaccaagccgaatcagcccgatgcgcgggcattgcggtggcgccctggatgccgtcgacgccggattgccggcgcggacgcgccagcgggacccatcggcgtcgcgttcgccggttgagcccggggtgagcccagacattcgatgtgcccaacaccatccgccacagcccaattgatgtggcactctatgcatgcctatccccgaccaaccaccaccgcggcgacgcatcatgaccggaggcgaagatgccagtagaggcgcccagaccagcgcgccatctggaggtcgagcgcaagttcgacgtgatcgagtcgacggtgtcgccgtcgttcgagggcatcgccgcggtggttcgcgtcgagcagtcgccgacccagcagctcgacgcggtgtacttcgacacaccgtcgcacgacctggcgcgcaaccagatcaccttgcggcgccgcaccggcggcgccgacgccggctggcatctgaagctgccggccggacccgacaagcgcaccgagatgcgagcaccgctgtccgcatcaggcgacgctgtgccggccgagttgttggatgtggtgctggcgatcgtccgcgaccagccggttcagccggtcgcgcggatcagcactcaccgcgaaagccagatcctgtacggcgccgggggcgacgcgctggcggaattctgcaacgacgacgtcaccgcatggtcggccggggcattccacgccgctggtgcagcggacaacggccctgccgaacagcagtggcgcgaatgggaactggaactggtcaccacggatgggaccgccgataccaagctactggaccggctagccaaccggctgctcgatgccggtgccgcacctgccggccacggctccaaactggcgcgggtgctcggtgcgacctctcccggtgagctgcccaacggcccgcagccgccggcggatccagtacaccgcgcggtgtccgagcaagtcgagcagctgctgctgtgggatcgggccgtgcgggccgacgcctatgacgccgtgcaccagatgcgagtgacgacccgcaagatccgcagcttgctgacggattcccaggagtcgtttggcctgaaggaaagtgcgtgggtcatcgatgaactgcgtgagctggccgatgtcctgggcgtagcccgggacgccgaggtactcggtgaccgctaccagcgcgaactggacgcgctggcgccggagctggtacgcggccgggtgcgcgagcgcctggtagacggggcgcggcggcgataccagaccgggctgcggcgatcactgatcgcattgcggtcgcagcggtacttccgtctgctcgacgctctagacgcgcttgtgtccgaacgcgcccatgccacttctggggaggaatcggcaccggtaaccatcgatgcggcctaccggcgagtccgcaaagccgcaaaagccgcaaagaccgccggcgaccaggcgggcgaccaccaccgcgacgaggcattgcacctgatccgcaagcgcgcgaagcgattacgctacaccgcggcggctactggggcggacaatgtgtcacaagaagccaaggtcatccagacgttgctaggcgatcatcaagacagcgtggtcagccgggaacatctgatccagcaggccatagccgcgaacaccgccggcgaggacaccttcacctacggtctgctctaccaacaggaagccgacttggccgagcgctgccgggagcagcttgaagccgcgctgcgcaaactcgacaaggcggtccgcaaagcacgggattgagcccgccaggggcggacgagttggcctgtaagccggattctgttccgcgccgccacagccaagctaacggcggcacggcggcgaccatccatctggacacaccgttaccgggtgcctcgagcggcctacccgcaggctcgggcgagcaaccctcaagcgcctgcgcggccgcactttcggtgcggccttcttggccttgcttcgggtggggtttgcctagccaccccggtcacccggaatgctggtgcgctcttaccgcaccgtttcacccttgccaccacgaggatggcggtctgttttctgtggcactttcccgcgagtcacctcggattgccgttagcaatcaccctgctctgtgaagtccggactttcctcgactcgacgctgaacctcgtgaatccacacaagccctacgcgagccgcggccgcccagccaactcatccgcgacgaccacgctaccccgctgggcggtgtcgcggccagtgtgaccgctggacgacacggctagtcggacagccgatccggcgggcagtccttatcgtggactggtgacacggtgggacaaacgcgtcgactccggcgactgggacgccatcgctgccgaggtcagcgagtacggtggcgcactgctacctcggctgatcacccccggcgaggccgcccggctgcgcaagctgtacgccgacgacggcctgtttcgctcgacggtcgatatggcatccaagcggtacggcgccgggcagtatcgatatttccatgccccctatcccgagtgatcgagcgtctcaagcaggcgctgtatcccaaactgctgccgatagcgcgcaactggtgggccaaactgggccgggaggcgccctggccagacagccttgatgactggttggcgagctgtcatgccgccggccaaacccgatccacagcgctgatgttgaagtacggcaccaacgactggaacgccctacaccaggatctctacggcgagttggtgtttccgctgcaggtggtgatcaacctgagcgatccggaaaccgactacaccggcggcgagttcctgcttgtcgaacagcggcctcgcgcccaatcccggggtaccgcaatgcaacttccgcagggacatggttatgtgttcacgacccgtgatcggccggtgcggactagccgtggctggtcggcatctccagtgcgccatgggctttcgactattcgttccggcgaacgctatgccatggggctgatctttcacgacgcagcctgattgcacgccatctatagatagcctgtctgattcaccaatcgcaccgacgatgccccatcggcgtagaactcggcgatgctcagcgatgccagatcaagatgcaaccgatataggacgcccgacccggcatccaacgccagccgcaacaacattttgatcggcgtgacatgtgacaccaccagcaccgtcgcgccttcgtagccaacgatgatccgatcacgtccccgccgaacccgccgcagcacgtcgtcgaagctttccccacccgggggcgtgatgctggtgtcctgcagccagcgacggtgcagctcgggatcgcgttctgcggcctccgcgaacgtcagcccctcccaggcgccgaagtcggtctcgaccaggtcgtcatcgacgaccacgtccagggccagggctctggcggcggtcaccgcggtgtcgtaagcccgctgtagcggcgaggagaccaccgcagcgatcccgccgcgccgcgccagatacccggccgccgcaccaacctggcgccaccccacctcgttcaaccccgggttgccgcgccccgaatagcggcgttgctccgacagctccgtctgcccgtggcgcaacaaaagtagtcgggtgggtgtaccgcgggcgccggtccagccgggagatgtcggtgactcggtcgcaacgattttggcaggatccgcatccgccgcagccgattgcgcggcggcgtccatcgcgtcattggccaaccggtctgcatacgtgttccgggcacgcggaacccactcgtagttgatcctgcgaaactgggacgccaacgcctgagcctggacatagagcttcagcagatccgggtgcttgaccttccaccgcccggacatctgctccaccaccagcttggagtccatcagcaccgcggcctcggtggcacctagtttcacggcgtcgtccaaaccggctatcaggccgcggtattcggcgacgttgttcgtcgcccggccgatcgcctgcttggactcggccagcacggtggagtgatcggcggtccacaccaccgcgccgtatccggccggtccgggattgccccgcgatccgccgtcggcttcgatgacaactttcactcctcaaatccttcgagccgcaacaagatcgctccgcattccgggcagcgcaccacttcatcctcggcggccgccgagatctgggccagctcgccgcggccgatctcgatccggcaggcaccacatcgatgaccttgcaaccgcccggcccctggcccgcctccggcccgctgtctttcgtagagccccgcaagctcgggatcaagtgtcgccgtcagcatgtcgcgttgcgatgaatgttggtgccgggcttggtcgatttcggcaagtgcctcgtccaaagcctgctgggcggcggccaggtcggcccgcaacgcttggagcgcccgcgactcggcggtctgttgagcctgcagctcctcgcggcgttccagcacctccagcagggcatcttccaaactggcttgacggcgttgcaagctgtcgagctcgtgctgcagatcagccaattgcttggcgtccgttgcacccgaagtgagcaacgaccggtcccggtcgccacgcttacgcaccgcatcgatctccgactcaaaacgcgacacctggccgtccaagtcctccgccgcgattcgcagggccgccatcctgtcgttggcggcgttgtgctcggcctgcacctgctggtaagccgcccgctgcggcagatgggtagcccgatgcgcgatccgggtcagctcagcatccagcttcgccaattccagtagcgaccgttgctgtgccactccggctttcatgcctgatctctcccagtttcgtgatcgaggttccacgggtcggtgcagatggtgcacacacgcaccggcagcgacgcgccgaaatgagaccgcaacacttcggcggcctggccgcaccacgggaattcgcttgcccaatgcgcgacgtcgatcagggccacttgcgaagctcggcaatgctcgtcggctggatgatgtcgcagatcggccgtaacgtacgcttgcacgtccgcggcggccacggtggcaagcaacgagtccccggcgccgccgcagaccgcgacccgcgacaccagcaggtcgggatccccggcggcgcgcacaccggtcgcagtcggcggcaacgcggcctccagacgggcaacaaaggtgcgcagcggttcgggttttggcagtctgccaatccggcctaacccgctgccgaccggcggtggtaccagcgcgaagatgtcgaatgccggctcctcgtaagggtgcgcggcgcgcatcgccgccaacacctcggcgcgcgctcgtgcgggtgcgacgacctcgacccggtcctcggccacccgttcgacggtaccgacgctgcctatggcgggcgacgccccgtcgtgcgccaggaactgcccggtacccgcgacactccagctgcagtgcgagtagtcgccgatatggccggcaccggcctcaaagaccgctgcccgcaccgcctctgagttctcgcgcggcacatagatgacccacttgtcgagatcggccgctccgggcaccgggtcgagaacggcgtcgacggtcagaccaacagcgtgtgccagcgcgtcggacacacccggcgacgccgagtcggcgttggtgtgcgcggtaaacaacgagcgaccggtccggatcaggcggtgcaccagcacaccctttggcgtgttggccgcgaccgtatcgaccccacgcagtaacaacgggtggtgcaccaatagcagtccggcctggggaacctggtccaccaccgccggcgtcgcgtccaccgcaacggtcaccgaatccaccacgtcgtcggggtcgccgcacaccagacccaccgaatcccacgactgggcaagccgcggcgggtaggcctggtccagcacgtcgatgacatcggccagccgcacactcatcggcgtcctccacgctttgcccactcggcgatcgccgccaccagcacgggccactccgggcgcaccgccgcccgcaggtaccgcgcgtccaggccgacgaaggtgtcaccgcggcgcaccgcaattcctttgctctgcaaatagtttcgtaatccgtcagcatcggcgatgttgaacagtacgaaaggggccgcaccatcgaccacctcggcacccaccgatctcagtccggccaccatctccgcgcgcagcgccgtcaaccgcaccgcatcggctgcggcagcggcgaccgcccggggggcgcagcaagcagcgatggccgtcagttgcaatgttcccaacggccagtgcgctcgctgcacggtcaaccgagccagcacgtctggcgagccgagcgcgtagcccacccgcaatccggccagcgaccacgttttcgtcaagctacggagcaccagcacatcgggcagcgagtcatcggccaacgattgcggctcgccgggaacccaatcagcgaacgcctcgtcgaccaccaggatgcgtcccggccggcgtaactcgagcagctgctcgcggaggtgcagcaccgaggtggggttggtcggattacccacgacgacaaggtcggcgtcgtcaggcacgtgcgcggtgtccagcacgaacggcggctttaggacaacatggtgcgccgtgattccggcagcgctcaaggctatggccggctcggtgaacgcgggcacgacgattgctgcccgcaccggacttaggttgtgcagcaatgcgaatccctccgccgccccgacgagcgggagcacttcgtcacgggttctgccatgacgttcagcgaccgcgtcttgcgcccggtgcacatcgtcggtgctcggatagcgggccagctccggcagcagcgcggcgagctgccggaccaaccattccgggggccggtcatggcggacgttgacggcgaagtccagcacgccgggcgcgacatcctgatcaccgtggtagcgcgccgcggcaagcgggctagtgtctagactcgccacagcgtcaaacagtagtgggccggtgtgcgggccaagaatccagagcaccgccgacgcgttgtctacgcggcgacaaccgcgacatcacaggcagctaacagggcgtcggcggtgatgatcgtcaggccaagcagctgtgcctgggcgatgagcacacggtcgaatggatgtcgatggtgatccggaagctctgcggtgcgcagtgtgtgcgtggtcaactgacagcggcgacgtgccgcagcggcgcattcgatcgggcacgtaagaagccgatggctcgggcggcgggagcttgccgaggcggtagttgatcgcgatctcccaggcactggcggccgacaagagaatgctgttgcggacgtcctgaacaatcgcccgtgtttcgttgacggcatccgcagccaaacgtgggtgtcgatgaggtagcgcttcaccggtgaaagcgttcgagcacgtcgtctgacaacggagcgtccaaatcgtcgggcacgcggtacacgccatggtcaatgcctaaccgccgagtctcatgaggatgcagcggcacaagctttgctaccggctcgccgcggcgggcaatctcaacctctgcccgccgtagacgagccgcagcagctcggacaggcgtgtcttcgcctcgtgaacgccgacccgcttcgcaggcgcccagactttcgcgtcgaccacctgctcaccaaacttcgcgatcatcgcctgataccacagcgccaacgggtagcggtttgtccaaccgcttcgtcaacgacaatgggatcgtgaccgacacgaccgcgagcgggaccaattgcccgcctcctccacgcgccgccgcacggcgcgcatcgtcgccgggtgaatcgccgcagctggtgatcttcgatctggacggcacgctgaccgactcggcgcgcggaatcgtatccagcttccgacacgcgctcaaccacatcggtgccccagtacccgaaggcgacctggccactcacatcgtcggcccgcccatgcatgagacgctgcgcgccatggggctcggcgaatccgccgaggaggcgatcgtagcctaccgggccgactacagcgcccgcggttgggcgatgaacagcttgttcgacgggatcgggccgctgctggccgacctgcgcaccgccggtgtccggctggccgtcgccacctccaaggcagagccgaccgcacggcgaatcctgcgccacttcggaattgagcagcacttcgaggtcatcgcgggcgcgagcaccgatggctcgcgaggcagcaaggtcgacgtgctggcccacgcgctcgcgcagctgcggccgctacccgagcggttggtgatggtcggcgaccgcagccacgacgtcgacggggcggccgcgcacggcatcgacacggtggtggtcggctggggctacgggcgcgccgactttatcgacaagacctccaccaccgtcgtgacgcatgccgccacgattgacgagctgagggaggcgctaggtgtctgatccgctgcacgtcacattcgtttgtacgggcaacatctgccggtcgccaatggccgagaagatgttcgcccaacagcttcgccaccgtggcctgggtgacgcggtgcgagtgaccagtgcgggcaccgggaactggcatgtaggcagttgcgccgacgagcgggcggccggggtgttgcgagcccacggctaccctaccgaccaccgggccgcacaagtcggcaccgaacacctggcggcagacctgttggtggccttggaccgcaaccacgctcggctgttgcggcagctcggcgtcgaagccgcccgggtacggatgctgcggtcattcgacccacgctcgggaacccatgcgctcgatgtcgaggatccctactatggcgatcactccgacttcgaggaggtcttcgccgtcatcgaatccgccctgcccggcctgcacgactgggtcgacgaacgtctcgcgcggaacggaccgagttgatgccccgcctagcgttcctgctgcggcccggctggctggcgttggccctggtcgtggtcgcgttcacctacctgtgctttacggtgctcgcgccgtggcagctgggcaagaatgccaaaacgtcacgagagaaccagcagatcaggtattccctcgacaccccgccggttccgctgaaaacccttctaccacagcaggattcgtcggcgccggacgcgcagtggcgccgggtgacggcaaccggacagtaccttccggacgtgcaggtgctggcccgactgcgcgtggtggagggggaccaggcgtttgaggtgttggccccattcgtggtcgacggcggaccaaccgtcctggtcgaccgtggatacgtgcggccccaggtgggctcgcacgtaccaccgatcccccgcctgccggtgcagacggtgaccatcaccgcgcggctgcgtgactccgaaccgagcgtggcgggcaaagacccattcgtcagagacggcttccagcaggtgtattcgatcaataccggacaggtcgccgcgctgaccggagtccagctggctgggtcctatctgcagttgatcgaagaccaacccggcgggctcggcgtgctcggcgttccgcatctagatcccgggccgttcctgtcctatggcatccaatggatctcgttcggcattctggcaccgatcggcttgggctatttcgcctacgccgagatccgggcgcgccgccgggaaaaagcggggtcgccaccaccggacaagccaatgacggtcgagcagaaactcgctgaccgctacggccgccggcggtaaaccaacatcacggccaataccgcagcccccgcctggaccacccgcgacagcaccacggcgcggcgcagatcggccaccttgggcgaccggccgtcgcccaaggtgggccggatctgcaactcatggtggtaccgggtgggcccacccagccgcacgtcaagcgccccagcaaacgccgcctcgacgacaccggcgttggggctgggatggcgggcggcgtcgcgccgccaggcccgtaccgcaccgcggggcgacccaccgaccaccggcgcgcagatcaccaccagcaccgccgtcgcccgtgcgccaacatagttggcccagtcatccaatcgtgctgcagcccaaccgaatcggagataacgcggcgagcggtagccgatcatcgagtccagggtgttgatggcacgatatcccagcaccgcaggcacgccgctcgaagccgcccacagcagcggcaccacctgggcgtcggcggtgttttcggccaccgactccagcgcggcacgcgtcaggcccgggccgcccagctgggccgggtcacgcccgcacagcgacggcagcagccgtcgcgccgcctcgacatcgtcgcgctccaacaggtccgatatctggcggccggtgcgcgccagcgaagttccgcccagcgctgcccaggtggccgtcgcggtggccgccacgggccaggacctgccgggtagccgctgcagtgccgcgccgagcaagcccaccgcgccgaccagcaggccgacgtgtaccgcaccggcgacccggccgtcacggtaggtgatctgctccagcttggcggccgcccgaccgaacagggccaccggatgacctcgtttggggtcgccgaacacgacgtcgagcaggcagccgatcagcacgccgacggccctggtctgccaggtcgatgcaaacactccggcagcgtcgcacacgtggtctacgctcagctatttatgacctcatacggcagctatccacgatgaagcggccagctacccgggttgccgacctgttgaacccggcggcaatgttgttgccggcagcgaatgtcatcatgcagctggcagtgccgggtgtcgggtatggcgtgctggaaagcccggtggacagcggcaacgtctacaagcatccgttcaagcgggcccggaccaccggcacctacctggcggtggcgaccatcgggacggaatccgaccgagcgctgatccggggtgccgtggacgtcgcgcaccggcaggttcggtcgacggcctcgagcccagtgtcctataacgccttcgacccgaagttgcagctgtgggtggcggcgtgtctgtaccgctacttcgtggaccagcacgagtttctgtacggcccactcgaagatgccaccgccgacgccgtctaccaagacgccaaacggttagggaccacgctgcaggtgccggaggggatgtggccgccggaccgggtcgcgttcgacgagtactggaagcgctcgcttgatgggctgcagatcgacgcgccggtgcgcgagcatcttcgcggggtggcctcggtagcgtttctcccgtggccgttgcgcgcggtggccgggccgttcaacctgtttgcgacgacgggattcttggcaccggagttccgcgcgatgatgcagctggagtggtcacaggcccagcagcgtcgcttcgagtggttactttccgtgctacggttagccgaccggctgattccgcatcgggcctggatcttcgtttaccagctttacttgtgggacatgcggtttcgcgcccgacacggccgccgaatcgtctgatagagcccggccgagtgtgagcctgacagcccgacaccggcggcgtgtgtcgcgtcgccaggttcacgctcggcgatctagagccgccgaaaacctacttctgggttgcctcccgaatcaacgtgctgatctgctcgagcagctcacgcatatcggcgcgcatcgcatccaccgcggcatacaggtcggccttggtcgccggcagctggtccgacgtcattggccgcaccggcggtgctgtctgtcgcgccgcgctgtcgctttgaaacccaggtcgctcacccacgaccacgacactgccatatccggcgccccgccgacaacgaagcacagctagccggtgggcgcggacgggatcgaaccgccgaccgctggtgtgtaaaaccagagctctaccgctgagctacgcgcccatgaccgccgcaggctacacgccttgcggccaagcacccaaaaccttaggccgtaagcgccgccagagcgtcggtccacagccgctgatcgcgaacttcacccggctgcttcatctcggcgaaccgaatgatccctgaccgatcgaccacaaaggtgccccggttagcgatgccggcctgctcgttgaagacgccgtaggcctgactgaccgcgccgtgtggccagaagtccgacaacagcggaaacgtgaatccgctctgcgtcgcccagatcttgtgagtgggtggcgggcccaccgaaatcgctagcgcggcgctgtcgtcgttctcaaactcgggcaggtgatcacgcaactggtccagctcgccctggcagatgcccgtgaacgccaacggaaagaacaccaacagcacgttctttgcaccccggtagccgcgcagggtgacaagctgctgattctggtcgcgcaacgtgaagtcaggggcggtggctccgacgttcagcatcagcgcttgccagcccgcgatttcggctgtaccaatctgctggcgctccagttgcccagattgaccgacgaggtcggcatcagcccagctgtgggcgccgcctcggcaatctcggcgggcaatacatggccgggctggccggtcttgggcgtcaccacccaaatcacaccgtcctcggcgagcgggccgatcgcatccatcagggtgtccaccaaatcgccgtcgccatcacgccaccacaacaggacgacatcgatgacctcgtcggtgtcttcatcgagcaactctcccccgcacgcttcttcgatggccgcgcggatgtcgtcgtcggtgtcttcgtcccagccccattcctggataagttggtctcgttggatgcccaatttgcgggcgtagttcgaggcgtgatccgccgcgaccaccgtggaacctccttcagtctccgcgggccatgtgcacaccgtcgcgatgggcattatcgtcgcacagccagaaccggtccacccgcccgcctcagaaggcggccacgcacattgtcaatgcctttgtcttggtgtcgttgagccgatcaacccgccggttgaattccgctgtcgacgcgtgcgcaccgatggcatttgccaccgcgcgggccgcgtcgacatatgcgttgagcgcatcccccagttgcgcggacagcgcggcgctcagactgcctgagaccgtcgaggcactgttgttgagcgcgtcgatggccggaccttcggtcggcccggtgttgcggccctgattgaacgcggccacgtaggcgttcaccttgtcgatggcgtccttgctggtggccgccagcgcgtcacacgaggtgcgaatcgccttggtcgtcagcgattgttggcgctgcgactcccggatgctcgacgtcgccgccgaagccgacaccgacgcggacaccgacgagcggtaggccggtgcgacgttggtgtcgggcatggccgtaccgtcggtgacagtggtacatccgacgatccccatcagcagcagcgcgatgcagccgagcgccagggcgcctcgcctggggagctcccccccgtgcctgcgaggcacggcgcgccatccgatgagcacggcatgtgaggttacctggtcgcagcgcgaccgcgctggccgtggtgtgtcgcgcatccgcagaaccgagcggagtgcggctatccgccgccgacgccggtgcggcacgatagggggacgaccatctaaacagcacgcaagcggaagcccgccacctacaggagtagtgcgttgaccaccgatttcgcccgccacgatctggcccaaaactcaaacagcgcaagcgaacccgaccgagttcgggtgatccgcgagggtgtggcgtcgtatttgcccgacattgatcccgaggagacctcggagtggctggagtcctttgacacgctgctgcaacgctgcggcccgtcgcgggcccgctacctgatgttgcggctgctagagcgggccggcgagcagcgggtggccatcccggcattgacgtctaccgactatgtcaacaccatcccgaccgagctggagccgtggttccccggcgacgaagacgtcgaacgtcgttatcgagcgtggatcagatggaatgcggccatcatggtgcaccgtgcgcaacgaccgggtgtgggcgtgggtggccatatctcgacctacgcgtcgtccgcggcgctctatgaggtcggtttcaaccacttcttccgcggcaagtcgcacccgggcggcggcgatcaggtgttcatccagggccacgcttccccgggaatctacgcgcgcgccttcctcgaagggcggttgaccgccgagcaactcgacggattccgccaggaacacagccatgtcggcggcgggttgccgtcctatccgcacccgcggctcatgcccgacttctgggaattccccaccgtgtcgatgggtttgggcccgctcaacgccatctaccaggcacggttcaaccactatctgcatgaccgcggtatcaaagacacctccgatcaacacgtgtggtgttttttgggcgacggcgagatggacgaacccgagagccgtgggctggcccacgtcggcgcgctggaaggcttggacaacttgaccttcgtgatcaactgcaatctgcagcgactcgacggcccggtgcgcggcaacggcaagatcatccaggagctggagtcgttcttccgcggtgccggctggaacgtcatcaaggtggtgtggggccgcgaatgggatgccctgctgcacgccgaccgcgacggtgcgctggtgaatttaatgaatacaacacccgatggcgattaccagacctataaggccaacgacggcggctacgtgcgtgaccacttcttcggccgcgacccacgcaccaaggcgctggtggagaacatgagcgaccaggatatctggaacctcaaacggggcggccacgattaccgcaaggtttacgccgcctaccgcgccgccgtcgaccacaagggacagccgacggtgatcctggccaagaccatcaaaggctacgcgctgggcaagcatttcgaaggacgcaatgccacccaccagatgaaaaaactgaccctggaagaccttaaggagtttcgtgacacgcagcggattccggtcagcgacgcccagcttgaagagaatccgtacctgccgccctactaccaccccggcctcaacgccccggagattcgttacatgctcgaccggcgccgggccctcgggggctttgttcccgagcgcaggaccaagtccaaagcgctgaccctgccgggtcgcgacatctacgcgccgctgaaaaagggctctgggcaccaggaggtggccaccaccatggcgacggtgcgcacgttcaaagaagtgttgcgcgacaagcagatcgggccgcggatagtcccgatcattcccgacgaggcccgcaccttcgggatggactcctggttcccgtcgctaaagatctataaccgcaatggccagctgtataccgcggttgacgccgacctgatgctggcctacaaggagagcgaagtcgggcagatcctgcacgagggcatcaacgaagccgggtcggtgggctcgttcatcgcggccggcacctcgtatgcgacgcacaacgaaccgatgatccccatttacatcttctactcgatgttcggcttccagcgcaccggcgatagcttctgggccgcggccgaccagatggctcgagggttcgtgctcggggccaccgccgggcgcaccaccctgaccggtgagggcctgcaacacgccgacggtcactcgttgctgctggccgccaccaacccggcggtggttgcctacgacccggccttcgcctacgaaatcgcctacatcgtggaaagcggactggccaggatgtgcggggagaacccggagaacatcttcttctacatcaccgtctacaacgagccgtacgtgcagccgccggagccggagaacttcgatcccgagggcgtgctgcggggtatctaccgctatcacgcggccaccgagcaacgcaccaacaaggcgcagatcctggcctccggggtagcgatgcccgcggcgctgcgggcagcacagatgctggccgccgagtgggatgtcgccgccgacgtgtggtcggtgaccagttggggcgagctaaaccgcgacggggtggccatcgagaccgagaagctccgccaccccgatcggccggcgggcgtgccctacgtgacgagagcgctggagaatgctcggggcccggtgatcgcggtgtcggactggatgcgcgcggtccccgagcagatccgaccgtgggtgccgggcacatacctcacgttgggcaccgacgggttcggcttttccgacactcggcccgccgctcgccgctacttcaacaccgacgccgaatcccaggtggtcgcggttttggaggcgttggcgggcgacggcgagatcgacccatcggtgccggtcgcggccgcccgccagtaccggatcgacgacgtggcggctgcgcccgagcagaccacggatcccggtcccggggcctaacgccggcgagccgaccgcctttggccgaatcttccagaaatctggcgtagcttttaggagtgaacgacaatcagttggctccagttgcccgcccgaggtcgccgctcgaactgctggacactgtgcccgattcgctgctgcggcggttgaagcagtactcgggccggctggccaccgaggcagtttcggccatgcaagaacggttgccgttcttcgccgacctagaagcgtcccagcgcgccagcgtggcgctggtggtgcagacggccgtggtcaacttcgtcgaatggatgcacgacccgcacagtgacgtcggctataccgcgcaggcattcgagctggtgccccaggatctgacgcgacggatcgcgctgcgccagaccgtggacatggtgcgggtcaccatggagttcttcgaagaagtcgtgcccctgctcgcccgttccgaagagcagttgaccgccctcacggtgggcattttgaaatacagccgcgacctggcattcaccgccgccacggcctacgccgatgcggccgaggcacgaggcacctgggacagccggatggaggccagcgtggtggacgcggtggtacgcggcgacaccggtcccgagctgctgtcccgggcggccgcgctgaattgggacaccaccgcgccggcgaccgtactggtgggaactccggcgcccggtccaaatggctccaacagcgacggcgacagcgagcgggccagccaggatgtccgcgacaccgcggctcgccacggccgcgctgcgctgaccgacgtgcacggcacctggctggtggcgatcgtctccggccagctgtcgccaaccgagaagttcctcaaagacctgctggcagcattcgccgacgccccggtggtcatcggccccacggcgcccatgctgaccgcggcgcaccgcagcgctagcgaggcgatctccgggatgaacgccgtcgccggctggcgcggagcgccgcggcccgtgctggctagggaacttttgcccgaacgcgccctgatgggcgacgcctcggcgatcgtggccctgcataccgacgtgatgcggcccctagccgatgccggaccgacgctcatcgagacgctagacgcatatctggattgtggcggcgcgattgaagcttgtgccagaaagttgttcgttcatccaaacacagtgcggtaccggctcaagcggatcaccgacttcaccgggcgcgatcccacccagccacgcgatgcctatgtccttcgggtggcggccaccgtgggtcaactcaactatccgacgccgcactgaagcatcgacagcaatgccgtgtcatagattccctcgccggtcagagggggtccagcaggggccccggaaagataccaggggcgccgtcggacggaaagtgatccagacaacaggtcgcgggacgatctcaaaaacatagcttacaggcccgttttgttggttatatacaaaaacctaagacgaggttcataatctgttacaccgcgcaaaaccgtcttcacagtgttctcttagacacgtgattgcgttgctcgcacccggacagggttcgcaaaccgagggaatgttgtcgccgtggcttcagctgcccggcgcagcggaccagatcgcggcgtggtcgaaagccgctgatctagatcttgcccggctgggcaccaccgcctcgaccgaggagatcaccgacaccgcggtcgcccagccattgatcgtcgccgcgactctgctggcccaccaggaactggcgcgccgatgcgtgctcgccggcaaggacgtcatcgtggccggccactccgtcggcgaaatcgcggcctacgcaatcgccggtgtgatagccgccgacgacgccgtcgcgctggccgccacccgcggcgccgagatggccaaggcctgcgccaccgagccgaccggcatgtctgcggtgctcggcggcgacgagaccgaggtgctgagtcgcctcgagcagctcgacttggtcccggcaaaccgcaacgccgccggccagatcgtcgctgccggccggctgaccgcgttggagaagctcgccgaagacccgccggccaaggcgcgggtgcgtgcactgggtgtcgccggagcgttccacaccgagttcatggcgcccgcacttgacggctttgcggcggccgcggccaacatcgcaaccgccgaccccaccgccacgctgctgtccaaccgcgacgggaagccggtgacatccgcggccgcggcgatggacaccctggtctcccagctcacccaaccggtgcgatgggacctgtgcaccgcgacgctgcgcgaacacacagtcacggcgatcgtggagttcccccccgcgggcacgcttagcggtatcgccaaacgcgaacttcggggggttccggcacgcgccgtcaagtcacccgcagacctggacgagctggcaaacctataaccgcggactcggccagaacaaccacatacccgtcagttcgatttgtacacaacatattacgaagggaagcatgctgtgcctgtcactcaggaagaaatcattgccggtatcgccgagatcatcgaagaggtaaccggtatcgagccgtccgagatcaccccggagaagtcgttcgtcgacgacctggacatcgactcgctgtcgatggtcgagatcgccgtgcagaccgaggacaagtacggcgtcaagatccccgacgaggacctcgccggtctgcgtaccgtcggtgacgttgtcgcctacatccagaagctcgaggaagaaaacccggaggcggctcaggcgttgcgcgcgaagattgagtcggagaaccccgatgccgttgccaacgttcaggcgaggcttgaggccgagtccaagtgagtcagccttccaccgctaatggcggtttccccagcgttgtggtgaccgccgtcacagcgacgacgtcgatctcgccggacatcgagagcacgtggaagggtctgttggccggcgagagcggcatccacgcactcgaagacgagttcgtcaccaagtgggatctagcggtcaagatcggcggtcacctcaaggatccggtcgacagccacatgggccgactcgacatgcgacgcatgtcgtacgtccagcggatgggcaagttgctgggcggacagctatgggagtccgccggcagcccggaggtcgatccagaccggttcgccgttgttgtcggcaccggtctaggtggagccgagaggattgtcgagagctacgacctgatgaatgcgggcggcccccggaaggtgtccccgctggccgttcagatgatcatgcccaacggtgccgcggcggtgatcggtctgcagcttggggcccgcgccggggtgatgaccccggtgtcggcctgttcgtcgggctcggaagcgatcgcccacgcgtggcgtcagatcgtgatgggcgacgccgacgtcgccgtctgcggcggtgtcgaaggacccatcgaggcgctgcccatcgcggcgttctccatgatgcgggccatgtcgacccgcaacgacgagcctgagcgggcctcccggccgttcgacaaggaccgcgacggctttgtgttcggcgaggccggtgcgctgatgctcatcgagacggaggagcacgccaaagcccgtggcgccaagccgttggcccgattgctgggtgccggtatcacctcggacgcctttcatatggtggcgcccgcggccgatggtgttcgtgccggtagggcgatgactcgctcgctggagctggccgggttgtcgccggcggacatcgaccacgtcaacgcgcacggcacggcgacgcctatcggcgacgccgcggaggccaacgccatccgcgtcgccggttgtgatcaggccgcggtgtacgcgccgaagtctgcgctgggccactcgatcggcgcggtcggtgcgctcgagtcggtgctcacggtgctgacgctgcgcgacggcgtcatcccgccgaccctgaactacgagacacccgatcccgagatcgaccttgacgtcgtcgccggcgaaccgcgctatggcgattaccgctacgcagtcaacaactcgttcgggttcggcggccacaatgtggcgcttgccttcgggcgttactgaagcacgacatcgcgggtcgcgaggcccgaggtgggggtccccccgcttgcgggggcgagtcggaccgatatggaaggaacgttcgcaagaccaatgacggagctggttaccgggaaagcctttccctacgtagtcgtcaccggcatcgccatgacgaccgcgctcgcgaccgacgcggagactacgtggaagttgttgctggaccgccaaagcgggatccgtacgctcgatgacccattcgtcgaggagttcgacctgccagttcgcatcggcggacatctgcttgaggaattcgaccaccagctgacgcggatcgaactgcgccggatgggatacctgcagcggatgtccaccgtgctgagccggcgcctgtgggaaaatgccggctcacccgaggtggacaccaatcgattgatggtgtccatcggcaccggcctgggttcggccgaggaactggtcttcagttacgacgatatgcgcgctcgcggaatgaaggcggtctcgccgctgaccgtgcagaagtacatgcccaacggggccgccgcggcggtcgggttggaacggcacgccaaggccggggtgatgacgccggtatcggcgtgcgcatccggcgccgaggccatcgcccgtgcgtggcagcagattgtgctgggagaggccgatgccgccatctgcggcggcgtggagaccaggatcgaagcggtgcccatcgccgggttcgctcagatgcgcatcgtgatgtccaccaacaacgacgaccccgccggtgcatgccgcccattcgacagggaccgcgacggctttgtgttcggcgagggcggcgcccttctgttgatcgagaccgaggagcacgccaaggcacgtggcgccaacatcctggcccggatcatgggcgccagcatcacctccgatggcttccacatggtggccccggaccccaacggggaacgcgccgggcatgcgattacgcgggcgattcagctggcgggcctcgcccccggcgacatcgaccacgtcaatgcgcacgccaccggcacccaggtcggcgacctggccgaaggcagggccatcaacaacgccttgggcggcaaccgaccggcggtgtacgcccccaagtctgccctcggccactcggtgggcgcggtcggcgcggtcgaatcgatcttgacggtgctcgcgttgcgcgatcaggtgatcccgccgacactgaatctggtaaacctcgatcccgagatcgatttggacgtggtggcgggtgaaccgcgaccgggcaattaccggtatgcgatcaataactcgttcggattcggcggccacaacgtggcaatcgccttcggacggtactaaaccccagcgttacgcgacaggagacctgcgatgacaatcatggcccccgaggcggttggcgagtcgctcgacccccgcgatccgctgttgcggctgagcaacttcttcgacgacggcagcgtggaattgctgcacgagcgtgaccgctccggagtgctggccgcggcgggcaccgtcaacggtgtgcgcaccatcgcgttctgcaccgacggcaccgtgatgggcggcgccatgggcgtcgaggggtgcacgcacatcgtcaacgcctacgacactgccatcgaagaccagagtcccatcgtgggcatctggcattcgggtggtgcccggctggctgaaggtgtgcgggcgctgcacgcggtaggccaggtgttcgaagccatgatccgcgcgtccggctacatcccgcagatctcggtggtcgtcggtttcgccgccggcggcgccgcctacggaccggcgttgaccgacgtcgtcgtcatggcgccggaaagccgggtgttcgtcaccgggcccgacgtggtgcgcagcgtcaccggcgaggacgtcgacatggcctcgctcggtgggccggagacccaccacaagaagtccggggtgtgccacatcgtcgccgacgacgaactcgatgcctacgaccgtgggcgccggttggtcggattgttctgccagcaggggcatttcgatcgcagcaaggccgaggccggtgacaccgacatccacgcgctgctgccggaatcctcgcgacgtgcctacgacgtgcgtccgatcgtgacggcgatcctcgatgcggacacaccgttcgacgagttccaggccaattgggcgccgtcgatggtggtcgggctgggtcggctgtcgggtcgcacggtgggtgtactggccaacaacccgctacgcctgggcggctgcctgaactccgaaagcgcagagaaggcagcgcgtttcgtgcggctgtgcgacgcgttcgggattccgctggtggtggtggtcgatgtgccgggctatctgcccggtgtcgaccaggagtggggtggcgtggtgcgccgtggcgccaagttgctgcacgcgttcggcgagtgcaccgttccgcgggtcacgctggtcacccgaaagacctacggcggggcatacattgcgatgaactcccggtcgttgaacgcgaccaaggtgttcgcctggccggacgccgaggtcgcggtgatgggcgctaaggcggccgtcggcatcctgcacaagaagaagttggccgccgctccggagcacgaacgcgaagcgctgcacgaccagttggccgccgagcatgagcgcatcgccggcggggtcgacagtgcgctggacatcggtgtggtcgacgagaagatcgacccggcgcatactcgcagcaagctcaccgaggcgctggcgcaggctccggcacggcgcggccgccacaagaacatcccgctgtagttctgaccgcgagcagacgcagaatcgcacgcgcgaggtccgcgccgtgcgattctgcgtctgctcgccagttatccccagcggtggctggtcaacgcgaggcgctcctcgcatgctcggacggtgcctaccgacgcgctaacaattctcgagaaggccggcgggttcgccaccaccgcgcaattgctcacggtcatgacccgccaacagctcgacgtccaagtgaaaaacggcggcctcgttcgcgtttggtacggggtctacgcggcacaagagccggacctgttgggccgcttggcggctctcgatgtgttcatgggggggcacgccgtcgcgtgtctgggcaccgccgccgcgttgtatggattcgacacggaaaacaccgtcgctatccatatgctcgatcccggagtaaggatgcggcccacggtcggtctgatggtccaccaacgcgtcggtgcccggctccaacgggtgtcaggtcgtctcgcgaccgcgcccgcatggactgccgtggaggtcgcacgacagttgcgccgcccgcgggcgctggccaccctcgacgccgcactacggtcaatgcgctgcgctcgcagtgaaattgaaaacgccgttgctgagcagcgaggccgccgaggcatcgtcgcggcgcgcgaactcttacccttcgccgacggacgcgcggaatcggccatggagagcgaggctcggctcgtcatgatcgaccacgggctgccgttgcccgaacttcaatacccgatacacggccacggtggtgaaatgtggcgagtcgacttcgcctggcccgacatgcgtctcgcggccgaatacgaaagcatcgagtggcacgcgggaccggcggagatgctgcgcgacaagacacgctgggccaagctccaagagctcgggtggacgattgtcccgattgtcgtcgacgatgtcagacgcgaacccggccgcctggcggcccgcatcgcccgccacctcgaccgcgcgcgtatggccggctgaccgctggtgagcagacgcagagtcgcactgcggccggcgcagtgcgactctgcgtctgctcgcgctcaacggctgaggaactccttagccacggcgactacgcgctcgcgatcccgtggcaccagaccgatccgggtccggcggtcgaggatatcgtccacatccagcgccccctcatgggtcaccgcgtattcgaactccgcccgggtcacgtcgatgccgtcggcgaccggctcggtgggccgctcacatgtggcggcggcagcgacgttggccgcctcggccccgtaccgcgccaccagcgactcgggcaatccggcgcccgatccgggggccggcccagggttcgccggtgcgccgatcagcggcaggttgcgagtgcggcacttcgcggctcgcaggtgtcgcagcgtgatggcgcgattcagcacatcctctgccatgtagcggtattccgtcagcttgccgccgaccacactgatcacgcccgacggcgattcaaaaacagcgtggtcacgcgaaacgtcggcggtgcggccctggacaccagcaccgccggtgtcgattagcggccgcaatcccgcataggcaccgatgacatccttggtgccgaccgccgtccccaatgcggtgttcaccgtatccagcaggaacgtgatctcttccgaagacggttgtggcacatcgggaatcgggccgggtgcgtcttcgtcggtcagcccgagatagatccggcccagctgctcgggcatggcgaacacgaagcggttcagctcaccggggatcggaatggtcagcgcggcagtcggattggcaaacgacttcgcgtcgaagaccagatgtgtgccgcggctggggcgtagcctcagggacgggtcgatctcacccgcccacacgcccgccgcgttgatgacggcacgcgccgacagcgcgaacgactgccgggtgcgccggtcggtcaactccaccgaagtgccggtgacattcgacgcgcccacgtaagtgaggatgcgggcgccgtgctgggccgcggtgcgcgcgacggccatgaccagccgggcgtcgtcgatcaattgcccgtcgtacgcgagcagaccaccgtcgaggccgtcccgccgaacggtgggagcaatctccaccacccgtgacgccgggattcggcgcgatcggggcaacgtcgccgccggcgtacccgctagcacccgcaaagcgtcgccggccaggaaaccggcacgcaccaacgcccgcttggtgtgacccatcgacggcaacaacgggaccagttgcggcatggcatgcacgagatgaggagcgttgcgtgtcatcaggattccgcgttcgacggcgctgcgccgggcgatgcccacgttgccgctggccagatagcgcagaccgccgtgcaccaacttcgagctccagcggctggtgccgaacgccagatcatgcttttccaccaaggccaccgtcagaccgcgggtggcagcatctaaggcaatgccaacaccggtaatgccgccgcctatcacgatgacgtcgagtgcgccaccgtcggccagtgcggtcaggtcggcggagcgacgcgccgcgttgagtgcagccgagtggggcatcagcacaaatatccgttcagtgcgtgggtaagttcggtggccagcgcggcggaatcgaggatcgaatcgacgatgtccgcggactggatggtcgactgggcgatcagcaacaccatggtcgccagtcgacgagcgtcgccggagcgcacactgcccgaccgctgcgccactgtcagccgggcggccaacccctcgatcaggacctgctggctggtgccgaggcgctcggtgatgtacaccctggccagctccgagtgcatgaccgacatgatcagatcgtcaccccgcaaccggtcggccaccgcgacaatctgctttaccaacgcttcccggtcgtccccgtcgaggggcacctcccgcagcacgtcggcgatatggctggtcagcatggacgccatgatcgaccgggtgtccggccagcgacggtatacggtcgggcggctcacgcccgcgcgccgggcgatctcggcaagtgtcacccggtccacgccgtaatcgacgacgcagctcgccgctgcccgcaggatacgaccaccggtatccgcgcggtcattactcattgacagcatgtgtaatactgtaacgcgtgactcaccgcgaggaactccttccaccgatgaaatgggacgcgtggggagatcccgccgcggccaagccactttctgatggcgtccggtcgttgctgaagcaggttgtgggcctagcggactcggagcagcccgaactcgaccccgcgcaggtgcagctgcgcccgtccgccctgtcgggggcagacca

SEQ ID NO: 25 - DNA, 2540 bp, Homo sapiens
gaaaaggtggacaagtcctattttcaagagaagatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtattgtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaactctatagtaggcatgttatgttgttcttattaccctgattgaatgtgtgatgtgaactgactttaagtaatcaggattgaattccattagcatttgctaccaagtaggaaaaaaaatgtacatggcagtgttttagttggcaatataatctttgaatttcttgatttttcagggtattagctgtattatccattttttttactgttatttaattgaaaccatagactaagaataagaagcatcatactataactgaacacaatgtgtattcatagtatactgatttaatttctaagtgtaagtgaattaatcatctggattttttattcttttcagataggcttaacaaatggagctttctgtatataaatgtggagattagagttaatctccccaatcacataatttgttttgtgtgaaaaaggaataaattgttccatgctggtggaaagatagagattgtttttagaggttggttgttgtgttttaggattctgtccattttcttgtaaagggataaacacggacgtgtgcgaaatatgtttgtaaagtgatttgccattgttgaaagcgtatttaatgatagaatactatcgagccaacatgtactgacatggaaagatgtcagagatatgttaagtgtaaaatgcaagtggcgggacactatgtatagtctgagccagatcaaagtatgtatgttgttaatatgcatagaacgagagatttggaaagatatacaccaaactgttaaatgtggtttctcttcggggagggggggattgggggaggggccccagaggggttttagaggggccttttcactttcgacttttttcattttgttctgttcggattttttataagtatgtagaccccgaagggttttatgggaactaacatcagtaacctaacccccgtgactatcctgtgctcttcctagggagctgtgttgtttcccacccaccacccttccctctgaacaaatgcctgagtgctggggcactttg

SEQ ID NO: 26 - RNA, 103 nt, Homo sapiens
agcuccuauaacaaaagucuguugcuuguguuucacauuuuggauuuccuaauauaauguucucuuuuuagaaaagguggacaaguccuauuuucaagagaag

SEQ ID NO: 27 - RNA, 28 nt, Homo sapiens
ggauuuccuaauauaauguucucuuuuu

SEQ ID NO: 28 - DNA, 1619 bp, Homo sapiens
ccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcctgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttc

(19) United States
(12) Patent Application Publication
(10) Pub. No.: US 2005/0142545 A1
(43) Pub. Date: Jun. 30, 2005

(54) Methods for identifying small molecules that bind specific rna structural motifs

(76) Inventors: Michael Morgan Conn, New York, NY (US); Matthew Pelligrini, Bedminster, NJ (US); Seongwoo Hwang, Edison, NJ (US); Young-Choon Moon, Belle Mead, NJ (US); Neil Almstead, Holmdel, NJ (US)

Correspondence Address: JONES DAY, 222 EAST 41ST ST, NEW YORK, NY, 10017, US

(21) Appl. No.: 10/475,026

(22) PCT Filed: Apr. 11, 2002
(86) PCT No.: PCT/US02/11758

Related U.S. Application Data
(60) Provisional application No. 60/282,966, filed on Apr. 11, 2001.

(51) Int. Cl. C12Q 1/68

(52) U.S. Cl. 435/6

(57) ABSTRACT

The present invention relates to a method for screening and identifying test compounds that bind to a preselected target ribonucleic acid (“RNA”). Direct, non-competitive binding assays are advantageously used to screen bead-based libraries of compounds for those that selectively bind to a preselected target RNA. Binding of target RNA molecules to a particular test compound is detected using any physical method that measures the altered physical property of the target RNA bound to a test compound. The structure of the test compound attached to the labeled RNA is also determined. The methods used will depend, in part, on the nature of the library screened. The methods of the present invention provide a simple, sensitive assay for high-throughput screening of libraries of compounds to identify pharmaceutical leads.

This application claims the benefit of U.S. Provisional Application No. 60/282,966, filed Apr. 11, 2001, which is incorporated herein by reference in its entirety.

### 1. INTRODUCTION

The present invention relates to a method for screening and identifying test compounds that bind to a preselected target ribonucleic acid (“RNA”). Direct, non-competitive binding assays are advantageously used to screen bead-based libraries of compounds for those that selectively bind to a preselected target RNA. Binding of target RNA molecules to a particular test compound is detected using any method that measures the altered physical property of the target RNA bound to a test compound. The methods of the present invention provide a simple, sensitive assay for high-throughput screening of libraries of compounds to identify pharmaceutical leads.

### 2. BACKGROUND OF THE INVENTION

Protein-nucleic acid interactions are involved in many cellular functions, including transcription, RNA splicing, mRNA decay, and mRNA translation. Readily accessible synthetic molecules that can bind with high affinity to specific sequences of single- or double-stranded nucleic acids have the potential to interfere with these interactions in a controllable way, making them attractive tools for molecular biology and medicine. Successful approaches for blocking function of target nucleic acids include using duplex-forming antisense oligonucleotides (Miller, 1996, Progress in Nucl. Acid Res. & Mol. Biol. 52:261-291; Ojwang & Rando, 1999, Achieving antisense inhibition by oligodeoxynucleotides containing N₇ modified 2′-deoxyguanosine using tumor necrosis factor receptor type 1, METHODS: A Companion to Methods in Enzymology 18:244-251) and peptide nucleic acids (“PNA”) (Nielsen, 1999, Current Opinion in Biotechnology 10:71-75), which bind to nucleic acids via Watson-Crick base-pairing. Triplex-forming anti-gene oligonucleotides can also be designed (Ping et al., 1997, RNA 3:850-860; Aggarwal et al., 1996, Cancer Res. 56:5156-5164; U.S. Pat. No. 5,650,316), as well as pyrrole-imidazole polyamide oligomers (Gottesfeld et al., 1997, Nature 387:202-205; White et al., 1998, Nature 391:468-471), which are specific for the major and minor grooves of a double helix, respectively.

In addition to synthetic nucleic acids (i.e., antisense, ribozymes, and triplex-forming molecules), there are examples of natural products that interfere with deoxyribonucleic acid (“DNA”) or RNA processes such as transcription or translation. For example, certain carbohydrate-based host cell factors, calicheamicin oligosaccharides, interfere with the sequence-specific binding of transcription factors to DNA and inhibit transcription in vivo (Ho et al., 1994, Proc. Natl. Acad. Sci. USA 91:9203-9207; Liu et al., 1996, Proc. Natl. Acad. Sci. USA 93:940-944). Certain classes of known antibiotics have been characterized and were found to interact with RNA. For example, the antibiotic thiostreptone binds tightly to a 60-mer from ribosomal RNA (Cundliffe et al., 1990, in The Ribosome: Structure, Function & Evolution (Schlessinger et al., eds.) American Society for Microbiology, Washington, D.C. pp. 479-490). Bacterial resistance to various antibiotics often involves methylation at specific rRNA sites (Cundliffe, 1989, Ann. Rev. Microbiol. 43:207-233). Aminoglycosidic aminocyclitol (aminoglycoside) antibiotics and peptide antibiotics are known to inhibit group I intron splicing by binding to specific regions of the RNA (von Ahsen et al., 1991, Nature (London) 353:368-370). Some of these same aminoglycosides have also been found to inhibit hammerhead ribozyme function (Stage et al., 1995, RNA 1:95-101). In addition, certain aminoglycosides and other protein synthesis inhibitors have been found to interact with specific bases in 16S rRNA (Woodcock et al., 1991, EMBO J. 10:3099-3103). An oligonucleotide analog of the 16S rRNA has also been shown to interact with certain aminoglycosides (Purohit et al., 1994, Nature 370:659-662). A molecular basis for hypersensitivity to aminoglycosides has been found to be located in a single base change in mitochondrial rRNA (Hutchin et al., 1993, Nucleic Acids Res. 21:4174-4179). Aminoglycosides have also been shown to inhibit the interaction between specific structural RNA motifs and the corresponding RNA binding protein. Zapp et al. (Cell, 1993, 74:969-978) has demonstrated that the aminoglycosides neomycin B, lividomycin A, and tobramycin can block the binding of Rev, a viral regulatory protein required for viral gene expression, to its viral recognition element in the IIB (or RRE) region of HIV RNA. This blockage appears to be the result of competitive binding of the antibiotics directly to the RRE RNA structural motif.

Single stranded sections of RNA can fold into complex tertiary structures consisting of local motifs such as loops, bulges, pseudoknots, guanosine quartets and turns (Chastain & Tinoco, 1991, Progress in Nucleic Acid Res. & Mol. Biol. 41:131-177; Chow & Bogdan, 1997, Chemical Reviews 97:1489-1514; Rando & Hogan, 1998, Biologic activity of guanosine quartet forming oligonucleotides in “Applied Antisense Oligonucleotide Technology” Stein. & Krieg (eds) John Wiley and Sons, New York, pages 335-352). Such structures can be critical to the activity of the nucleic acid and affect functions such as regulation of mRNA transcription, stability, or translation (Weeks & Crothers, 1993, Science 261:1574-1577). The dependence of these functions on the native three-dimensional structural motifs of single-stranded stretches of nucleic acids makes it difficult to identify or design synthetic agents that bind to these motifs using general, simple-to-use sequence-specific recognition rules for the formation of double- and triple-helical nucleic acids used in the design of antisense and ribozyme type molecules. Approaches to screening generally involve competitive assays designed to identify compounds that disrupt the interaction between a target RNA and a physiological, host cell factor(s) that had been previously identified to specifically interact with that particular target RNA. In general, such assays require the identification and characterization of the host cell factor(s) deemed to be required for the function of the target RNA. Both the target RNA and its preselected host cell binding partner are used in a competitive format to identify compounds that disrupt or interfere with the two components in the assay.

Citation or identification of any reference in Section 2 of this application is not an admission that such reference is available as prior art to the present invention.

### 3. SUMMARY OF THE INVENTION

The present invention relates to methods for identifying compounds that bind to preselected target elements of nucleic acids including, but not limited to, specific RNA sequences, RNA structural motifs, and/or RNA structural elements. The specific target RNA sequences, RNA structural motifs, and/or RNA structural elements are used as targets for screening small molecules and identifying those that directly bind these specific sequences, motifs, and/or structural elements. For example, methods are described in which a preselected target RNA having a detectable label is used to screen a library of test compounds, preferably under physiologic conditions. Any complexes formed between the target RNA and a member of the library are identified using methods that detect the labeled target RNA bound to a test compound. In particular, the present invention relates to methods for using a target RNA having a detectable label to screen a bead-based library of test compounds. Compounds in the bead-based library that bind to the labeled target RNA will form a bead-based detectably labeled complex, which can be separated from the unbound beads and unbound target RNA in the liquid phase by a number of physical means, including, but not limited to, flow cytometry, affinity chromatography, manual batch mode separation, suspension of beads in electric fields, and microwave of the bead-based detectably labeled complex. The detectably labeled complex can then be identified by the label on the target RNA and removed from the uncomplexed, unlabeled test compounds in the library. The structure of the test compound complexed with the labeled RNA is then ascertained by de novo structure determination of the test compounds using, for example, mass spectrometry or nuclear magnetic resonance (“NMR”). The test compounds identified are useful for any purpose to which a binding reaction may be put, for example in assay methods, diagnostic procedures, cell sorting, as inhibitors of target molecule function, as probes, as sequestering agents and the like. In addition, small organic molecules which interact specifically with target RNA molecules may be useful as lead compounds for the development of therapeutic agents.

The methods described herein for the identification of compounds that directly bind to a particular preselected target RNA are well suited for high-throughput screening. The direct binding method of the invention offers advantages over drug screening systems for competitors that inhibit the formation of naturally-occurring RNA binding protein:target RNA complexes; i.e., competitive assays. The direct binding method of the invention is rapid and can be set up to be readily performed, e.g., by a technician, making it amenable to high throughput screening. The method of the invention also eliminates the bias inherent in the competitive drug screening systems, which require the use of a preselected host cell factor that may not have physiological relevance to the activity of the target RNA. Instead, the methods of the invention are used to identify any compound that can directly bind to specific target RNA sequences, RNA structural motifs, and/or RNA structural elements, preferably under physiologic conditions. As a result, the compounds so identified can inhibit the interaction of the target RNA with any one or more of the native host cell factors (whether known or unknown) required for activity of the RNA in vivo. The present invention may be understood more fully by reference to the detailed description and examples, which are intended to illustrate non-limiting embodiments of the invention.

### 3.1. Definitions

As used herein, a “target nucleic acid” refers to RNA, DNA, or a chemically modified variant thereof. In a preferred embodiment, the target nucleic acid is RNA. A target nucleic acid also refers to tertiary structures of the nucleic acids, such as, but not limited to loops, bulges, pseudoknots, guanosine quartets and turns. A target nucleic acid also refers to RNA elements such as, but not limited to, the HIV TAR element, internal ribosome entry site, “slippery site”, instability elements, and adenylate uridylate-rich elements, which are described in Section 4.1. Non-limiting examples of target nucleic acids are presented in Section 4.1 and Section 5.

As used herein, a “library” refers to a plurality of test compounds with which a target nucleic acid molecule is contacted. A library can be a combinatorial library, e.g., a collection of test compounds synthesized using combinatorial chemistry techniques, or a collection of unique chemicals of low molecular weight (less than 1000 daltons) that each occupy a unique three-dimensional space.

As used herein, a “label” or “detectable label” is a composition that is detectable, either directly or indirectly, by spectroscopic, photochemical, biochemical, immunochemical, or chemical means. For example, useful labels include radioactive isotopes (e.g., ³²P, ³⁵S, and 3H), dyes, fluorescent dyes, electron-dense reagents, enzymes and their substrates (e.g., as commonly used in enzyme-linked immunoassays, e.g., alkaline phosphatase and horse radish peroxidase), biotin, digoxigenin, or haptens and proteins for which antisera or monoclonal antibodies are available. Moreover, a label or detectable moiety can include an “affinity tag” that, when coupled with the target nucleic acid and incubated with a test compound or compound library, allows for the affinity capture of the target nucleic acid along with molecules bound to the target nucleic acid. One skilled in the art will appreciate that a affinity tag bound to the target nucleic acids has, by definition, a complimentary ligand coupled to a solid support that allows for its capture. For example, useful affinity tags and complimentary ligands include, but are not limited to, biotin-streptavidin, complimentary nucleic acid fragments (e.g., oligo dT-oligo dA, oligo T-oligo A, oligo dG-oligo dC, oligo G-oligo C), aptamer complexes, or haptens and proteins for which antisera or monoclonal antibodies are available. The label or detectable moiety is typically bound, either covalently, through a linker or chemical bound, or through ionic, van der Waals or hydrogen bonds to the molecule to be detected.

As used herein, a “dye” refers to a molecule that, when exposed to radiation, emits radiation at a level that is detectable visually or via conventional spectroscopic means. As used herein, a “visible dye” refers to a molecule having a chromophore that absorbs radiation in the visible region of the spectrum (i.e., having a wavelength of between about 400 nm and about 700 nm) such that the transmitted radiation is in the visible region and can be detected either visually or by conventional spectroscopic means. As used herein, an “ultraviolet dye” refers to a molecule having a chromophore that absorbs radiation in the ultraviolet region of the spectrum (i.e., having a wavelength of between about 30 nm and about 400 nm). As used herein, an “infrared dye” refers to a molecule having a chromophore that absorbs radiation in the infrared region of the spectrum (i.e., having a wavelength between about 700 nm and about 3,000 nm). A “chromophore” is the network of atoms of the dye that, when exposed to radiation, emits radiation at a level that is detectable visually or via conventional spectroscopic means. One of skill in the art will readily appreciate that although a dye absorbs radiation in one region of the spectrum, it may emit radiation in another region of the spectrum. For example, an ultraviolet dye may emit radiation in the visible region of the spectrum. One of skill in the art will also readily appreciate that a dye can transmit radiation or can emit radiation via fluorescence or phosphorescence.

The phrase “pharmaceutically acceptable salt(s),” as used herein includes but is not limited to salts of acidic or basic groups that may be present in test compounds identified using the methods of the present invention. Test compounds that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Test compounds that include an amino moiety may form pharmaceutically or cosmetically acceptable salts with various amino acids, in addition to the acids mentioned above. Test compounds that are acidic in nature are capable of forming base salts with various pharmacologically or cosmetically acceptable cations. Examples of such salts include alkali metal or alkaline earth metal salts and, particularly, calcium, magnesium, sodium lithium, zinc, potassium, and iron salts.

By “substantially one type of test compound,” as used herein, is meant that the assay can be performed in such a fashion that at some point, only one compound need be used in each reaction so that, if the result is indicative of a binding event occurring between the target RNA molecule and the test compound the test compound, can be easily identified.

### 4. DETAILED DESCRIPTION OF THE INVENTION

The present invention relates to methods for identifying compounds that bind to preselected target elements of nucleic acids, in particular, RNAs, including but not limited to preselected target RNA sequencing structural motifs, or structural elements. Methods are described in which a preselected target RNA having a detectable label is used to screen a library of test compounds. Any complexes formed between the target RNA and a member of the library are identified using methods that detect the labeled target RNA bound to a test compound. In particular, the present invention relates to methods for using a target RNA having a detectable label to screen a bead-based library of test compounds. Compounds in the bead-based library that bind to the labeled target RNA will form a bead-based detectably labeled complex, which can be separated from the unbound target RNA in the liquid phase by a number of physical means, such as, but not limited to, flow cytometry, affinity chromatography, manual batch mode separation, suspension of beads in electric fields, and microwave of the bead-based detectably labeled complex. The detectably labeled complex can then be identified by the label on the target RNA and removed from the uncomplexed, unlabeled test compounds in the library. The structure of the test compound attached to the labeled RNA is then ascertained by de novo structure determination of the test compounds using, for example, mass spectrometry or nuclear magnetic resonance (“NMR”).

Thus, the methods of the present invention provide a simple, sensitive assay for high-throughput screening of libraries of test compounds, in which the test compounds of the library that specifically bind a preselected target nucleic acid are easily distinguished from non-binding members of the library. The structures of the binding molecules are ascertained by de novo structure determination of the test compounds using, for example, mass spectrometry or nuclear magnetic resonance (“NMR”). The test compounds so identified are useful for any purpose to which a binding reaction may be put, for example in assay methods, diagnostic procedures, cell sorting, as inhibitors of target molecule function, as probes, as sequestering agents and lead compounds for development of therapeutics, and the like. Small organic compounds that are identified to interact specifically with the target RNA molecules are particularly attractive candidates as lead compounds for the development of therapeutic agents.

The assay of the invention reduces bias introduced by competitive binding assays which require the identification and use of a host cell factor (presumably essential for modulating RNA function) as a binding partner for the target RNA. The assays of the present invention are designed to detect any compound or agent that binds to the target RNA, preferably under physiologic conditions. Such agents can then be tested for biological activity, without establishing or guessing which host cell factor or factors is required for modulating the function and/or activity of the target RNA.

Section 4.1 describes examples of protein-RNA interactions that are important in a variety of cellular functions and several target RNA elements that can be used to identify test compounds. Compounds that inhibit these interactions by binding to the RNA and successfully competing with the natural protein or host cell factor that endogenously binds to the RNA may be important, e.g., in treating or preventing a disease or abnormal condition, such as an infection or unchecked growth. Section 4.2 describes detectable labels for target nucleic acids that are useful in the methods of the invention. Section 4.3 describes libraries of test compounds. Section 4.4 provides conditions for binding a labeled target RNA to a test compound of a library and detecting RNA binding to a test compound using the methods of the invention. Section 4.5 provides methods for separating complexes of target RNAs bound to a test compound from an unbound RNA. Section 4.6 describes methods for identifying test compounds that are bound to the target RNA. Section 4.7 describes a secondary, biological screen of test compounds identified by the methods of the invention to test the effect of the test compounds in vivo. Section 4.8 describes the use of test compounds identified by the methods of the invention for treating or preventing a disease or abnormal condition in mammals.

### 4.1. Biologically Important RNA-Host Cell Factor Interactions

Nucleic acids, and in particular RNAs, are capable of folding into complex tertiary structures that include bulges, loops, triple helices and pseudoknots, which can provide binding sites for host cell factors, such as proteins and other RNAs. RNA-protein and RNA-RNA interactions are important in a variety cellular functions, including transcription, RNA splicing, RNA stability and translation. Furthermore, the binding of such host cell factors to RNAs may alter the stability and translational efficiency of such RNAs, and according affect subsequent translation. For example, some diseases are associated with protein overproduction or decreased protein function. In this case, the identification of compounds to modulate RNA stability and translational efficiency will be useful to treat and prevent such diseases.

The methods of the present invention are useful for identifying test compounds that bind to target RNA elements in a high throughput screening assay of libraries of test compounds in solution. In particular, the methods of the present invention are useful for identifying a test compound that binds to a target RNA elements and inhibits the interaction of that RNA with one or more host cell factors in vivo. The molecules identified using the methods of the invention are useful for inhibiting the formation of a specific bound RNA:host cell factor complexes in vivo.

In some embodiments, test compounds identified by the methods of the invention are useful for increasing or decreasing the translation of messenger RNAs (“mRNAs”), e.g., protein production, by binding to one or more regulatory elements in the 5′ untranslated region, the 3′ untranslated region, or the coding region of the mRNA. Compounds that bind to mRNA can, inter alia, increase or decrease the rate of mRNA processing, alter its transport through the cell, prevent or enhance binding of the mRNA to ribosomes, suppressor proteins or enhancer proteins, or alter mRNA stability. Accordingly, compounds that increase or decrease mRNA translation can be used to treat or prevent disease. For example, diseases associated with protein overproduction, such as amyloidosis, or with the production of mutant proteins, such as Ras, can be treated or prevented by decreasing translation of the mRNA that codes for the overproduced protein, thus inhibiting production of the protein. Conversely, the symptoms of diseases associated with decreased protein function, such as hemophelia, may be treated by increasing translation of mRNA coding for the protein whose function is decreased, e.g., factor IX in some forms of hemophilia.

The methods of the invention can be used to identify compounds that bind to mRNAs coding for a variety of proteins with which the progression of diseases in mammals is associated. These mRNAs include, but are not limited to, those coding for amyloid protein and amyloid precursor protein; anti-angiogenic proteins such as angiostatin, endostatin, METH-1 and METH-2; apoptosis inhibitor proteins such as survivin, clotting factors such as Factor IX, Factor VIII, and others in the clotting cascade; collagens; cyclins and cyclin inhibitors, such as cyclin dependent kinases, cyclin D1, cyclin E, WAF1, cdk4 inhibitor, and MTS1; cystic fibrosis transmembrane conductance regulator gene (CFTR); cytokines such as IL-1, IL-2, IL-3, IL-4, IL-5, IL-6, IL-7, IL-8, IL-9, IL-10, IL-11, IL-12, IL-13, IL-14, IL-15, IL-16, IL-17 and other interleukins; hematopoetic growth factors such as erythropoietin (Epo); colony stimulating factors such as G-CSF, GM-CSF, M-CSF, SCF and thrombopoietin; growth factors such as BNDF, BMP, GGRP, EGF, FGF, GDNF, GGF, HGF, IGF-1, IGF-2, KGF, myotrophin, NGF, OSM, PDGF, somatotrophin, TGF-β, TGF-α and VEGF; antiviral cytokines such as interferons, antiviral proteins induced by interferons, TNF-α, and TNF-β; enzymes such as cathepsin K, cytochrome P-450 and other cytochromes, farnesyl transferase, glutathione-s transferases, heparanase, HMG CoA synthetase, N-acetyltransferase, phenylalanine hydroxylase, phosphodiesterase, ras carboxyl-terminal protease, telomerase and TNF converting enzyme; glycoproteins such as cadherins, e.g., N-cadherin and E-cadherin; cell adhesion molecules; selecting; transmembrane glycoproteins such as CD40; heat shock proteins; hormones such as 5-α reductase, atrial natriuretic factor, calcitonin, corticotrophin releasing factor, diuretic hormones, glucagon, gonadotropin, gonadotropin releasing hormone, growth hormone, growth hormone releasing factor, somatotropin, insulin, leptin, luteinizing hormone, luteinizing hormone releasing hormone, parathyroid hormone, thyroid hormone, and thyroid stimulating hormone; proteins involved in immune responses, including antibodies, CTLA4, hemagglutinin, MHC proteins, VLA-4, and kallikrein-kininogen-kinin system; ligands such as CD4; oncogene products such as sis, hst, protein tyrosine kinase receptors, ras, abl, mos, myc, fos, jun, H-ras, ki-ras, c-fms, bcl-2, L-myc, c-myc, gip, gsp, and HER-2; receptors such as bombesin receptor, estrogen receptor, GABA receptors, growth factor receptors including EGFR, PDGFR, FGFR, and NGFR, GTP-binding regulatory proteins, interleukin receptors, ion channel receptors, leukotriene receptor antagonists, lipoprotein receptors, opioid pain receptors, substance P receptors, retinoic acid and retinoid receptors, steroid receptors, T-cell receptors, thyroid hormone receptors, TNF receptors; tissue plasminogen activator; transmembrane receptors; transmembrane transporting systems, such as calcium pump, proton pump, Na/Ca exchanger, MRP1, MRP2, P170, LRP, and cMOAT; transferrin; and tumor suppressor gene products such as APC, brca1, brca2, DCC, MCC, MTS1, NF1, NF2, nm23, p53 and Rb. In addition to the eukaryotic genes listed above, the invention, as described, can be used to define molecules that interrupt viral, bacterial or fungal transcription or translation efficiencies and therefore form the basis for a novel anti-infectious disease therapeutic. Other target genes include, but are not limited to, those disclosed in Section 4.1 and Section 5.

The methods of the invention can be used to identify mRNA-binding test compounds for increasing or decreasing the production of a protein, thus treating or preventing a disease associated with decreasing or increasing the production of said protein, respectively. The methods of the invention may be useful for identifying test compounds for treating or preventing a disease in mammals, including cats, dogs, swine, horses, goats, sheep, cattle, primates and humans. Such diseases include, but are not limited to, amyloidosis, hemophilia, Alzheimer's disease, atherosclerosis, cancer, giantism, dwarfism, hypothyroidism, hyperthyroidism, inflammation, cystic fibrosis, autoimmune disorders, diabetes, aging, obesity, neurodegenerative disorders, and Parkinson's disease. Other diseases include, but are not limited to, those described in Section 4.1 and diseases caused by aberrant expression of the genes disclosed in Example 5. In addition to the eukaryotic genes listed above, the invention, as described, can be used to define molecules that interrupt viral, bacterial or fungal transcription or translation efficiencies and therefore form the basis for a novel anti-infectious disease therapeutic.

In other embodiments, test compounds identified by the methods of the invention are useful for preventing the interaction of an RNA, such as a transfer RNA (“tRNA”), an enzymatic RNA or a ribosomal RNA (“rRNA”), with a protein or with another RNA, thus preventing, e.g., assembly of an in vivo protein-RNA or RNA-RNA complex that is essential for the viability of a cell. The term “enzymatic RNA,” as used herein, refers to RNA molecules that are either self-splicing, or that form an enzyme by virtue of their association with one or more proteins, e.g., as in RNase P, telomerase or small nuclear ribonuclear protein particles. For example, inhibition of an interaction between rRNA and one or more ribosomal proteins may inhibit the assembly of ribosomes, rendering a cell incapable of synthesizing proteins. In addition, inhibition of the interaction of precursor rRNA with ribonucleases or ribonucleoprotein complexes (such as RNase P) that process the precursor rRNA prevent maturation of the rRNA and its assembly into ribosomes. Similarly, a tRNA:tRNA synthetase complex may be inhibited by test compounds identified by the methods of the invention such that tRNA molecules do not become charged with amino acids. Such interactions include, but are not limited to, rRNA interactions with ribosomal proteins, tRNA interactions with tRNA synthetase, RNase P protein interactions with RNase P RNA, and telomerase protein interactions with telomerase RNA.

In other embodiments, test compounds identified by the methods of the invention are useful for treating or preventing a viral, bacterial, protozoan or fungal infection. For example, transcriptional up-regulation of the genes of human immunodeficiency virus type 1 (“HIV-1”) requires binding of the HIV Tat protein to the HIV trans-activation response region RNA (“TAR RNA”). HIV TAR RNA is a 59-base stem-loop structure located at the 5′-end of all nascent HIV-1 transcripts (Jones & Peterlin, 1994, Annu. Rev. Biochem. 63:717-43). Tat protein is known to interact with uracil 23 in the bulge region of the stem of TAR RNA. Thus, TAR RNA is a potential binding target for test compounds, such as small peptides and peptide analogs that bind to the bulge region of TAR RNA and inhibit formation of a Tat-TAR RNA complex involved in HIV-1 upregulation (see Hwang et al., 1999 Proc. Natl. Acad. Sci. USA 96:12997-13002). Accordingly, test compounds that bind to TAR RNA are useful as anti-HIV therapeutics (Hamy et al., 1997, Proc. Natl. Acad. Sci. USA 94:3548-3553; Hamy et al., 1998, Biochemistry 37:5086-5095; Mei et al., 1998, Biochemistry 37:14204-14212), and therefore, are useful for treating or preventing AIDS.

The methods of the invention can be used to identify test compounds to treat or prevent viral, bacterial, protozoan or fungal infections in a patient. In some embodiments, the methods of the invention are useful for identifying compounds that decrease translation of microbial genes by interacting with mRNA, as described above, or for identifying compounds that inhibit the interactions of microbial RNAs with proteins or other ligands that are essential for viability of the virus or microbe. Examples of microbial target RNAs useful in the present invention for identifying antiviral, antibacterial, anti-protozoan and anti-fungal compounds include, but are not limited to, general antiviral and anti-inflammatory targets such as mRNAs of INFα, INFγ, RNAse L, RNAse L inhibitor protein, PKR, tumor necrosis factor, interleukins 1-15, and IMP dehydrogenase; internal ribosome entry sites; HIV-1 CT rich domain and RNase H mRNA; HCV internal ribosome entry site (required to direct translation of HCV mRNA), and the 3′-untranslated tail of HCV genomes; rotavirus NSP3 binding site, which binds the protein NSP3 that is required for rotavirus mRNA translation; HBV epsilon domain; Dengue virus 5′ and 3′ untranslated regions, including IRES; INFα, INFβ and INFγ; plasmodium falciparum mRNAs; the 16S ribosomal subunit ribosomal RNA and the RNA component of RNase P of bacteria; and the RNA component of telomerase in fungi and cancer cells. Other target viral and bacterial mRNAs include, but are not limited so, those disclosed in Section 5.

One of skill in the art will appreciate that, although such target RNAs are functionally conserved in various species (e.g., from yeast to humans), they exhibit nucleotide sequence and structural diversity. Therefore, inhibition of, for example, yeast telomerase by an anti-fungal compound identified by the methods of the invention might not interfere with human telomerase and normal human cell proliferation.

Thus, the methods of the invention can be used to identify test compounds that interfere with one or more target RNA interactions with host cell factors that are important for cell growth or viability, or essential in the life cycle of a virus, a bacterium, a protozoa or a fungus. Such test compounds and/or congeners that demonstrate desirable biologic and pharmacologic activity can be administered to a patient in need thereof in order to treat or prevent a disease caused by viral, bacterial, protozoan, or fungal infections. Such diseases include, but are not limited to, HIV infection, AIDS, human T-cell leukemia, SIV infection, FIV infection, feline leukemia, hepatitis A, hepatitis B, hepatitis C, Dengue fever, malaria, rotavirus infection, severe acute gastroenteritis, diarrhea, encephalitis, hemorrhagic fever, syphilis, legionella, whooping cough, gonorrhea, sepsis, influenza, pneumonia, tinea infection, candida infection, and meningitis.

Non-limiting examples of RNA elements involved in the regulation of gene expression, i.e., mRNA stability, translational efficiency via translational initiation and ribosome assembly, etc., include the HIV TAR element, internal ribosome entry site, “slippery site”, instability elements, and adenylate uridylate-rich elements, as discussed below.

### 4.1.1. HIV TAR Element

Transcriptional up-regulation of the genes of human immunodeficiency virus type 1 (“HIV-1”) requires binding of the HIV Tat protein to the HIV trans-activation response region RNA (“TAR RNA”), a 59-base stem-loop structure located at the 5′ end of all nascent HIV-1 transcripts (Jones & Peterlin, 1994, Annu. Rev. Biochem. 63:717-43). Tat protein is known to interact with uracil 23 in the bulge region of the stem of TAR RNA. Thus, TAR RNA is a useful binding target for test compounds, such as small peptides and peptide analogs that bind to the bulge region of TAR RNA and inhibit formation of a Tat-TAR RNA complex involved in HIV-1 up-regulation (see Hwang et al., 1999 Proc. Natl. Acad. Sci. USA 96:12997-13002). Accordingly, test compounds that bind to TAR RNA can be useful as anti-HIV therapeutics (Hamy et al., 1997, Proc. Natl. Acad. Sci. USA 94:3548-3553; Hamy et al., 1998, Biochemistry 37:5086-5095; Mei et al., 1998, Biochemistry 37:14204-14212), and therefore, are useful for treating or preventing AIDS.

### 4.1.2. Internal Ribosome Entry Site (“IRES”)

Internal ribosome entry sites (“IRES”) are found in the 5′ untranslated regions (“5“UTR”) of several mRNAs, and are thought to be involved in the regulation of translational efficiency. When the IRES element is present on an mRNA downstream of a translational stop codon, it directs ribosomal re-entry (Ghattas et al., 1991, Mol. Cell. Biol. 11:5848-5959), which permits initiation of translation at the start of a second open reading frame.

As reviewed by Jang et al., a large segment of the 5′ nontranslated region, approximately 400 nucleotides in length, promotes internal entry of ribosomes independent of the non-capped 5′ end of picornavirus mRNAs (mammalian plus-strand RNA viruses whose genomes serve as mRNA). This 400 nucleotide segment (IRES), maps approximately 200 nt down-stream from the 5′ end and is highly structured. IRES elements of different picornaviruses, although functionally similar in vitro and in vivo, are not identical in sequence or structure. However, IRES elements of the genera entero- and rhinoviruses, on the one hand, and cardio- and aphthoviruses, on the other hand, reveal similarities corresponding to phylogenetic kinship. All IRES elements contain a conserved Yn-Xm-AUG unit (Y, pyrimidine; X, nucleotide) which appears essential for IRES function. The IRES elements of cardio-, entero- and aphthoviruses bind a cellular protein, p57. In the case of cardioviruses, the interaction between a specific stem-loop of the IREs is essential for translation in vitro. The IRES elements of entero- and cardioviruses also bind the cellular protein, p52, but the significance of this interaction remains to be shown. The function of p57 or p52 in cellular metabolism is unknown. Since picornaviral IRES elements function in vivo in the absence of any viral gene products, is speculated that IRES-like elements may also occur in specific cellular mRNAs releasing them from cap-dependent translation (Jang et al., 1990, Enzyme 44(1-4):292-309).

### 4.1.3. “Slippery Site”

Programmed, or directed, ribosomal frameshifting, when ribosomes shift from one translation reading frame to another and synthesize two viral proteins from a single viral mRNA, is directed by a unique site in viral mRNAs called the “slippery site.” The slippery site directs ribosomal frameshifting in the −1 or +1 direction that causes the ribosome to slip by one base in the 5′ direction thereby placing the ribosome in the new reading frame to produce a new protein.

Programmed, or directed, ribosomal frameshifting is of particular value to viruses that package their plus strands, as it eliminates the need to splice their mRNAs and reduces the risk of packaging defective genomes and regulates the ratio of viral proteins synthesized. Examples of programmed translational frameshifting (both +1 and −1 shifts) have been identified in ScV systems (Lopinski et al., 2000, Mol. Cell. Biol. 20(4):1095-103, retroviruses (Falk et al., 1993, J. Virol. 67:273-6277; Jacks & Varmus, 1985, Science 230:1237-1242; Morikawa & Bishop, 1992, Virology 186:389-397; Nam et al., 1993, J. Virol. 67:196-203); coronaviruses (Brierley et al., 1987, EMBO J. 6:3779-3785; Herold & Siddell, 1993, Nucleic Acids Res. 21:5838-5842); giardiaviruses, which are also members of the Totiviridae (Wang et al., 1993, Proc. Natl. Acad. Sci. USA 90:8595-8599); two bacterial genes (Blinkowa & Walker, 1990, Nucleic Acids Res., 18:1725-1729; Craigen & Caskey, 1986, Nature 322:273); bacteriophage genes (Condron et al., 1991, Nucleic Acids Res. 19:5607-5612); astroviruses (Marczinke et al., 1994, J. Virol. 68:5588-5595); the yeast EST3 gene (Lundblad & Morris, 1997, Curr. Biol. 7:969-976); and the rat, mouse, Xenopus, and Drosophila ornithine decarboxylase antizymes (Matsufuji et al., 1995, Cell 80:51-60); and a significant number of cellular genes (Herold & Siddell, 1993, Nucleic Acids Res. 21:5838-5842).

Drugs targeted to ribosomal frameshifting minimize the problem of virus drug resistance because this strategy targets a host cellular process rather than one introduced into the cell by the virus, which minimizes the ability of viruses to evolve drug-resistant mutants. Compounds that target the RNA elements involved in regulating programmed frameshifting should have several advantages, including (a) any selective pressure on the host cellular translational machinery to adapt to the drugs would have to occur at the host evolutionary time scale, which is on the order of millions of years, (b) ribosomal frameshifting is not used to express any host proteins, and (c) altering viral frameshifting efficiencies by modulating the activity of a host protein minimizing the likelihood that the virus will acquire resistance to such inhibition by mutations in its own genome.

### 4.1.4. Instability Elements

“Instability elements” may be defined as specific sequence elements that promote the recognition of unstable mRNAs by cellular turnover machinery. Instability elements have been found within mRNA protein coding regions as well as untranslated regions.

Altering the control of stability of normal mRNAs may lead to disease. The alteration of mRNA stability has been implicated in diseases such as, but not limited to, cancer, immune disorders, heart disease, and fibrotic disorders.

There are several examples of mutations that delete instability elements which then result in stabilization of mRNAs that may be involved in the onset of cancer. In Burkitt's lymphoma, a portion of the c-myc proto-oncogene is translocated to an Ig locus, producing a form of the c-myc mRNA that is five times more stable (see, e.g., Kapstein et al., 1996, J. Biol. Chem. 271(31):18875-84). The highly oncogenic v-fos mRNA lacks the 3′ UTR adenylate uridylate rich element (“ARE”) that is found in the more labile and weakly oncogenic c-fos mRNA (see, e.g., Schiavi et al., 1992, Biochim Biophys Acta. 1114(2-3):95-106). Differences between the benign cervical lesions brought about by nonintegrated circular human papillomavirus type 16 and its integrated form, that lacks the 3′ UTR ARE and correlates with cervical carcinomas, may be a consequence of stabilizing the E6/E7 transcripts encoding oncogenic proteins. Integration of the virus results in deletion of the ARE instability element, resulting in stabilizion of the transcripts and over-expression of the proteins (see, e.g., Jeon & Lambert, 1995, Proc. Natl. Acad. Sci. USA 92(5):1654-8). Deletion of AREs from the 3′ UTR of the IL-2 and IL-3 genes promotes increased stabilization of these mRNAs, high expression of these proteins, and leads to the formation of cancerous cells (see, e.g., Stoecklin et al., 2000, Mol. Cell. Biol. 20(11):3753-63).

Mutations in trans-acting factors involved in mRNA turnover may also promote cancer. In monocytic tumors, the lymphokine GM-CSF mRNA is specifically stabilized as a consequence of an oncogenic lesion in a trans-acting factor that controls mRNA turnover rates. Furthermore, the normally unstable IL-3 transcript is inappropriately long-lived in mast tumor cells. Similarly, the labile GM-CSF mRNA is greatly stabilized in bladder carcinoma cells. See, e.g., Bickel et al., 1990, J. Immunol. 145(3):840-5.

The immune system is regulated by a large number of regulatory molecules that either activate or inhibit the immune response. It has now been clearly demonstrated that stability of the transcripts encoding these proteins are highly regulated. Altered regulation of these molecules leads to mis-regulation of this process and can result in drastic medical consequences. For example, recent results using transgenic mice have shown that mis-regulation of the stability of the important modulator TNFα mRNA leads to diseases such as, but not limited to, rheumatoid arthritis and a Crohn's-like liver disease. See, e.g., Clark, 2000, Arthritis Res. 2(3):172-4.

Smooth muscle in the heart is modulated by the β-adrenergic receptor, which in turn responds to the sympathetic neurotransmitter norepinephrine and the adrenal hormone epinephrine. Chronic heart failure is characterized by impairment of smooth muscle cells, which results, in part, from the more rapid decay of the β-adrenergic receptor mRNA. See, e.g., Ellis & Frielle T., 1999, Biochem. Biophys. Res. Commun. 258(3):552-8.

A large number of diseases result from over-expression of collagen. For example, cirrhosis results from damage to the liver as a consequence of cancer, viral infection, or alcohol abuse. Such damage causes mis-regulation of collagen expression, leading to the formation of large collagen deposits. Recent results indicate that the sizeable increase in collagen expression is largely attributable to stabilization of its mRNA. See, e.g., Lindquist et al., 2000, Am. J. Physiol. Gastrointest. Liver Physiol. 279(3):G471-6.

### 4.1.5. Adenylate Uridylate-Rich Elements (“ARE”)

Adenylate uridylate-rich elements (“ARE”) are found in the 3′ untranslated regions (“3′ UTR”) of several mRNAs, and involved in the turnover of mRNAs, such as but not limited to transcription factors, cytokines, and lymphokines. AREs may function both as stabilizing and destabilizing elements. ARE mRNAs are classified into five groups, depending on sequence (Bakheet et al., 2001, Nucl. Acids Res. 29(1):246-254). An ongoing database at the web site http://rc.kfshrc.edu.sa/ared contains ARE-containing mRNAs and their cluster groups, which is incorporated by reference in its entirety. The ARE motifs are classified as follows:

| | | |
|---|---|---|
| Group I | (AUUUAUUUAUUUAUUUAUUUA) | SEQ ID NO: 1 |
| Cluster | | |
| Group II | (AUUUAUUUAUUUAUUUA) stretch | SEQ ID NO: 2 |
| Cluster | | |
| Group III | (WAUUUAUUUAUUUAW) stretch | SEQ ID NO: 3 |
| Cluster | | |
| Group IV | (WWAUUUAUUUAWW) stretch | SEQ ID NO: 4 |
| Cluster | | |
| Group V | (WWWWAUUUAWWWW) stretch | SEQ ID NO: 5 |
| Cluster | | |

The ARE-mRNAs were clustered into five groups containing five, four, three and two pentameric repeats, while the last group contains only one pentamer within the 13-bp ARE pattern. Functional categories were assigned whenever possible according to NCBI-COG functional annotation (Tatusov et al., 2001, Nucleic Acids Research, 29(1): 22-28), in addition to the categories: inflammation, immune response, development/differentiation, using an extensive literature search.

Group I contains many secreted proteins including GM-CSF, IL-1, IL-11, IL-12 and Gro-β that affect the growth of hematopoietic and immune cells (Witsell & Schook, 1992, Proc. Natl. Acad. Sci. USA, 89:4754-4758). Although TNFα is both a pro-inflammatory and anti-tumor protein, there is experimental evidence that it can act as a growth factor in certain leukemias and lymphomas. (Liu et al., 2000, J. Biol. Chem. 275:21086-21093).

Unlike Group I, Groups II-V contain functionally diverse gene families comprising immune response, cell cycle and proliferation, inflammation and coagulation, angiogenesis, metabolism, energy, DNA binding and transcription, nutrient transportation and ionic homeostasis, protein synthesis, cellular biogenesis, signal transduction, and apoptosis (Bakheet et al., 2001, Nucl. Acids Res. 29(1):246-254).

Several groups have described ARE-binding proteins that influence the ARE-mRNA stability. Among the well-characterized proteins are the mammalian homologs of ELAV (embryonic lethal abnormal vision) proteins including AUF1, HuR and He1-N2 (Zhang et al., 1993, Mol. Cell. Biol. 13:7652-7665; Levine et al., 1993, Mol. Cell. Biol. 13:3494-3504: Ma et al., 1996, J. Biol. Chem. 271:8144-8151). The zinc-finger protein tristetraprolin has been identified as another ARE-binding protein with destabilizing activity on TNFα, IL-3 and GM-CSF mRNAs (Stoecklin et al., 2000, Mol. Cell. Biol. 20:3753-3763; Carballo et al., 2000, Blood 95:1891-1899).

Since ARE-containing genes are clearly important in biological systems, including but not limited to a number of the early response genes that regulate cell proliferation and responses to exogenous agents, the identification of compounds that bind to one or more of the ARE clusters and potentially modulate the stability of the target RNA can potentially be of value as a therapeutic.

### 4.2. Detectably Labeled Target RNAs

Target nucleic acids, including but not limited to RNA and DNA, useful in the methods of the present invention have a label that is detectable via conventional spectroscopic means or radiographic means. Preferably, target nucleic acids are labeled with a covalently attached dye molecule. Useful dye-molecule labels include, but are not limited to, fluorescent dyes, phosphorescent dyes, ultraviolet dyes, infrared dyes, and visible dyes. Preferably, the dye is a visible dye.

Useful labels in the present invention can include, but are not limited to, spectroscopic labels such as fluorescent dyes (e.g., fluorescein and derivatives such as fluorescein isothiocyanate (FITC) and Oregon Green™, rhodamine and derivatives (e.g., Texas red, tetramethylrhodimine isothiocynate (TRITC), bora-3a,4a-diaza-s-indacene (BODIPY®) and derivatives, etc.), digoxigenin, biotin, phycoerythrin, AMCA, CyDye™, and the like), radiolabels (e.g., ³H, ¹²⁵I, ³⁵S, ¹⁴C, ³²P, ³³P, etc.), enzymes (e.g., horse radish peroxidase, alkaline phosphatase etc.), spectroscopic colorimetric labels such as colloidal gold or colored glass or plastic (e.g. polystyrene, polypropylene, latex, etc.) beads, or nanoparticles—nanoclusters of inorganic ions with defined dimension from 0.1 to 1000 nm. The label may be coupled directly or indirectly to a component of the detection assay (e.g., the detection reagent) according to methods well known in the art. A wide variety of labels may be used, with the choice of label depending on sensitivity required, ease of conjugation with the compound, stability requirements, available instrumentation, and disposal provisions.

In one embodiment, nucleic acids that are labeled at one or more specific locations are chemically synthesized using phosphoramidite or other solution or solid-phase methods. Detailed descriptions of the chemistry used to form polynucleotides by the phosphoramidite method are well known (see, e.g., Caruthers et al., U.S. Pat. Nos. 4,458,066 and 4,415,732; Caruthers et al., 1982, Genetic Engineering 4:1-17; Users Manual Model 392 and 394 Polynucleotide Synthesizers, 1990, pages 6-1 through 6-22, Applied Biosystems, Part No. 901237; Ojwang, et al., 1997, Biochemistry, 36:6033-6045). The phosphoramidite method of polynucleotide synthesis is the preferred method because of its efficient and rapid coupling and the stability of the starting materials. The synthesis is performed with the growing polynucleotide chain attached to a solid support, such that excess reagents, which are generally in the liquid phase, can be easily removed by washing, decanting, and/or filtration, thereby eliminating the need for purification steps between synthesis cycles.

The following briefly describes illustrative steps of a typical polynucleotide synthesis cycle using the phosphoramidite method. First, a solid support to which is attached a protected nucleoside monomer at its 3′ terminus is treated with acid, e.g., trichloroacetic acid, to remove the 5′-hydroxyl protecting group, freeing the hydroxyl group for a subsequent coupling reaction. After the coupling reaction is completed an activated intermediate is formed by contacting the support-bound nucleoside with a protected nucleoside phosphoramidite monomer and a weak acid, e.g., tetrazole. The weak acid protonates the nitrogen atom of the phosphoramidite forming a reactive intermediate. Nucleoside addition is generally complete within 30 seconds. Next, a capping step is performed, which terminates any polynucleotide chains that did not undergo nucleoside addition. Capping is preferably performed using acetic anhydride and 1-methylimidazole. The phosphite group of the internucleotide linkage is then converted to the more stable phosphotriester by oxidation using iodine as the preferred oxidizing agent and water as the oxygen donor. After oxidation, the hydroxyl protecting group of the newly added nucleoside is removed with a protic acid, e.g., trichloroacetic acid or dichloroacetic acid, and the cycle is repeated one or more times until chain elongation is complete. After synthesis, the polynucleotide chain is cleaved from the support using a base, e.g., ammonium hydroxide or t-butyl amine. The cleavage reaction also removes any phosphate protecting groups, e.g., cyanoethyl. Finally, the protecting groups on the exocyclic amines of the bases and any protecting groups on the dyes are removed by treating the polynucleotide solution in base at an elevated temperature, e.g., at about 55° C. Preferably the various protecting groups are removed using ammonium hydroxide or t-butyl amine.

Any of the nucleoside phosphoramidite monomers can be labeled using standard phosphoramidite chemistry methods (Hwang et al., 1999, Proc. Natl. Acad. Sci. USA 96(23):12997-13002; Ojwang et al., 1997, Biochemistry. 36:6033-6045 and references cited therein). Dye molecules useful for covalently coupling to phosphoramidites preferably comprise a primary hydroxyl group that is not part of the dye's chromophore. Illustrative dye molecules include, but are not limited to, disperse dye CAS 4439-31-0, disperse dye CAS 6054-58-6, disperse dye CAS 4392-69-2 (Sigma-Aldrich, St. Louis, Mo.), disperse red, and 1-pyrenebutanol (Molecular Probes, Eugene, Oreg.). Other dyes useful for coupling to phosphoramidites will be apparent to those of skill in the art, such as fluoroscein, cy3, and cy5 fluorescent dyes, and may be purchased from, e.g., Sigma-Aldrich, St. Louis, Mo. or Molecular Probes, Inc., Eugene, Oreg.

In another embodiment, dye-labeled target RNA molecules are synthesized enzymatically using in vitro transcription (Hwang et al., 1999, Proc. Natl. Acad. Sci. USA 96(23): 12997-13002 and references cited therein). In this embodiment, a template DNA is denatured by heating to about 90° C. and an oligonucleotide primer is annealed to the template DNA, for example by slow-cooling the mixture of the denatured template and the primer from about 90° C. to room temperature. A mixture of ribonucleoside-5′-triphosphates capable of supporting template-directed enzymatic extension of the primed template (e.g., a mixture including GTP, ATP, CTP, and UTP), including one or more dye-labeled ribonucleotides (Sigma-Aldrich, St. Louis, Mo.), is added to the primed template. Next, a polymerase enzyme is added to the mixture under conditions where the polymerase enzyme is active, which are well-known to those skilled in the art. A labeled polynucleotide is formed by the incorporation of the labeled ribonucleotides during polymerase-mediated strand synthesis.

In yet another embodiment of the invention, nucleic acid molecules are end-labeled after their synthesis. Methods for labeling the 5′-end of an oligonucleotide include but are by no means limited to: (i) periodate oxidation of a 5′-to-5′-coupled ribonucleotide, followed by reaction with an amine-reactive label (Heller & Morisson, 1985, in Rapid Detection and Identification of Infectious Agents, D. T. Kingsbury and S. Falkow, eds., pp. 245-256, Academic Press); (ii) condensation of ethylenediamine with 5′-phosphorylated polynucleotide, followed by reaction with an amine-reactive label (Morrison, European Patent Application 232 967); (iii) introduction of an aliphatic amine substituent using an aminohexyl phosphite reagent in solid-phase DNA synthesis, followed by reaction with an amine reactive label (Cardullo et al., 1988, Proc. Natl. Acad. Sci. USA 85:8790-8794); and (iv) introduction of a thiophosphate group on the 5′-end of the nucleic acid, using phosphatase treatment followed by end-labeling with ATP-S and kinase, which reacts specifically and efficiently with maleimide-labeled fluorescent dyes (Czworkowski et al., 1991, Biochem. 30:4821-4830).

A detectable label should not be incorporated into a target nucleic acid at the specific binding site at which test compounds are likely to bind, since the presence of a covalently attached label might interfere sterically or chemically with the binding of the test compounds at this site. Accordingly, if the region of the target nucleic acid that binds to a host cell factor is known, a detectable label is preferably incorporated into the nucleic acid molecule at one or more positions that are spatially or sequentially remote from the binding region.

After synthesis, the labeled target nucleic acid can be purified using standard techniques known to those skilled in the art (see Hwang et al., 1999, Proc. Natl. Acad. Sci. USA 96(23): 12997-13002 and references cited therein). Depending on the length of the target nucleic acid and the method of its synthesis, such purification techniques include, but are not limited to, reverse-phase high-performance liquid chromatography (“reverse-phase HPLC”), fast performance liquid chromatography (“FPLC”), and gel purification. After purification, the target RNA is refolded into its native conformation, preferably by heating to approximately 85-95° C. and slowly cooling to room temperature in a buffer, e.g., a buffer comprising about 50 mM Tris-HCl, pH 8 and 100 mM NaCl.

In another embodiment, the target nucleic acid can also be radiolabeled. A radiolabel, such as, but not limited to, an isotope of phosphorus, sulfur, or hydrogen, may be incorporated into a nucleotide, which is added either after or during the synthesis of the target nucleic acid. Methods for the synthesis and purification of radiolabeled nucleic acids are well known to one of skill in the art. See, e.g., Sambrook et al., 1989, in Molecular Cloning: A Laboratory Manual, pp 10.2-10.70, Cold Spring Harbor Laboratory Press, and the references cited therein, which are hereby incorporated by reference in their entireties.

In another embodiment, the target nucleic acid can be attached to an inorganic nanoparticle. A nanoparticle is a cluster of ions with controlled size from 0.1 to 1000 run comprised of metals, metal oxides, or semiconductors including, but not limited to Ag₂S, ZnS, CdS, CdTe, Au, or TiO₂. Nanoparticles have unique optical, electronic and catalytic properties relative to bulk materials which can be adjusted according to the size of the particle. Methods for the attachment of nucleic acids are well know to one of skill in the art (see, e.g., Niemeyer, 2001, Angew. Chem. Int. Ed. 40: 4129-4158, International Patent Publication WO/0218643, and the references cited therein, the disclosures of which are hereby incorporated by reference in their entireties).

### 4.3. Libraries of Small Molecules

Libraries screened using the methods of the present invention can comprise a variety of types of test compounds on solid supports. In all of the embodiments described below, all of the libraries can be synthesized on solid supports or the compounds of the library can be attached to solid supports by linkers.

In some embodiments, the test compounds are nucleic acid or peptide molecules. In a non-limiting example, peptide molecules can exist in a phage display library. In other embodiments, types of test compounds include, but are not limited to, peptide analogs including peptides comprising non-naturally occurring amino acids, e.g., D-amino acids, phosphorous analogs of amino acids, such as α-amino phosphoric acids and α-amino phosphoric acids, or amino acids having non-peptide linkages, nucleic acid analogs such as phosphorothioates and PNAs, hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose. Libraries of polypeptides or proteins can also be used.

In a preferred embodiment, the combinatorial libraries are small organic molecule libraries, such as, but not limited to, benzodiazepines, isoprenoids, thiazolidinones, metathiazanones, pyrrolidines, morpholino compounds, and diazepindiones. In another embodiment, the combinatorial libraries comprise peptoids; random bio-oligomers; benzodiazepines; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; or carbohydrate libraries. Combinatorial libraries are themselves commercially available (see, e.g., Advanced ChemTech Europe Ltd., Cambridgeshire, UK; ASINEX, Moscow Russia; BioFocus plc, Sittingbourne, UK; Bionet Research (A division of Key Organics Limited), Camelford, UK; ChemBridge Corporation, San Diego, Calif.; ChemDiv Inc, San Diego, Calif.; ChemRx Advanced Technologies, South San Francisco, Calif.; ComGenex Inc., Budapest, Hungary; Evotec OAI Ltd, Abingdon, UK; IF LAB Ltd., Kiev, Ukraine; Maybridge plc, Cornwall, UK; PharmaCore, Inc., North. Carolina; SIDDCO Inc, Tucson, Ariz.; TimTec Inc, Newark, Del.; Tripos Receptor Research Ltd, Bude, UK; Toslab, Ekaterinburg, Russia).

In one embodiment, the combinatorial compound library for the methods of the present invention may be synthesized. There is a great interest in synthetic methods directed toward the creation of large collections of small organic compounds, or libraries, which could be screened for pharmacological, biological or other activity (Dolle, 2001, J. Comb. Chem. 3:477-517; Hall et al., 2001, ibid. 3:125-150; Dolle, 2000, ibid. 2:383-433; Dolle, 1999, ibid. 1:235-282); The synthetic methods applied to create vast combinatorial libraries are performed in solution or in the solid phase, i.e., on a solid support. Solid-phase synthesis makes it easier to conduct multi-step reactions and to drive reactions to completion with high yields because excess reagents can be easily added and washed away after each reaction step. Solid-phase combinatorial synthesis also tends to improve isolation, purification and screening. However, the more traditional solution phase chemistry supports a wider variety of organic reactions than solid-phase chemistry. Methods and strategies for the synthesis of combinatorial libraries can be found in A Practical Guide to Combinatorial Chemistry, A. W. Czarnik and S. H. Dewitt, eds., American Chemical Society, 1997; The Combinatorial Index, B. A. Bunin, Academic Press, 1998; Organic Synthesis on Solid Phase, F. Z. Dörwald, Wiley-VCH, 2000; and Solid-Phase Organic Syntheses, Vol. 1, A. W. Czarnik, ed., Wiley Interscience, 2001.

Combinatorial compound libraries of the present invention may be synthesized using apparatuses described in U.S. Pat. No. 6,358,479 to Frisina et al., U.S. Pat. No. 6,190,619 to Kilcoin et al., U.S. Pat. No. 6,132,686 to Gallup et al., U.S. Pat. No. 6,126,904 to Zuellig et al., U.S. Pat. No. 6,074,613 to Harness et al., U.S. Pat. No. 6,054,100 to Stanchfield et al., and U.S. Pat. No. 5,746,982 to Saneii et al. which are hereby incorporated by reference in their entirety. These patents describe synthesis apparatuses capable of holding a plurality of reaction vessels for parallel synthesis of multiple discrete compounds or for combinatorial libraries of compounds.

In one embodiment, the combinatorial compound library can be synthesized in solution. The method disclosed in U.S. Pat. No. 6,194,612 to Boger et al., which is hereby incorporated by reference in its entirety, features compounds useful as templates for solution phase synthesis of combinatorial libraries. The template is designed to permit reaction products to be easily purified from unreacted reactants using liquid/liquid or solid/liquid extractions. The compounds produced by combinatorial synthesis using the template will preferably be small organic molecules. Some compounds in the library may mimic the effects of non-peptides or peptides. In contrast to solid phase synthesize of combinatorial compound libraries, liquid phase synthesis does not require the use of specialized protocols for monitoring the individual steps of a multistep solid phase synthesis (Egner et al., 1995, J. Org. Chem. 60:2652; Anderson et al., 1995, J. Org. Chem. 60:2650; Fitch et al., 1994, J. Org. Chem. 59:7955; Look et al., 1994, J. Org. Chem. 49:7588; Metzger et al., 1993, Angew. Chem., Int. Ed. Engl. 32:894; Youngquist et al., 1994, Rapid Commun. Mass Spect. 8:77; Chu et al., 1996, J. Am. Chem. Soc. 117:5419; Brummel et al., 1994, Science 264:399; Stevanovic etal., 1993, Bioorg. Med. Chem. Lett. 3:431).

Combinatorial compound libraries useful for the methods of the present invention can be synthesized on solid supports. In one embodiment, a split synthesis method, a protocol of separating and mixing solid supports during the synthesis, is used to synthesize a library of compounds on solid supports (see Lam et al., 1997, Chem. Rev. 97:41-448; Ohlmeyer et al., 1993, Proc. Natl. Acad. Sci. USA 90:10922-10926 and references cited therein). Each solid support in the final library has substantially one type of test compound attached to its surface. Other methods for synthesizing combinatorial libraries on solid supports, wherein one product is attached to each support, will be known to those of skill in the art (see, e.g., Nefzi et al., 1997, Chem. Rev. 97:449-472 and U.S. Pat. No. 6,087,186 to Cargill et al. which are hereby incorporated by reference in their entirety).

As used herein, the term “solid support” is not limited to a specific type of solid support. Rather a large number of supports are available and are known to one skilled in the art. Solid supports include silica gels, resins, derivatized plastic films, glass beads, cotton, plastic beads, polystyrene beads, doped polystyrene beads (as described by Fenniri et al., 2000, J. Am. Chem. Soc. 123:8151-8152), alumina gels, and polysaccharides. A suitable solid support may be selected on the basis of desired end use and suitability for various synthetic protocols. For example, for peptide synthesis, a solid support can be a resin such as p-methylbenzhydrylamine (pMBHA) resin (Peptides International, Louisville, Ky.), polystyrenes (e.g., PAM-resin obtained from Bachem Inc., Peninsula Laboratories, etc.), including chloromethylpolystyrene, hydroxymethylpolystyrene and aminomethylpolystyrene, poly(dimethylacrylamide)-grafted styrene co-divinyl-benzene (e.g., POLYHIPE resin, obtained from Aminotech, Canada), polyamide resin (obtained from Peninsula Laboratories), polystyrene resin grafted with polyethylene glycol (e.g., TENTAGEL or ARGOGEL, Bayer, Tubingen, Germany) polydimethylacrylamide resin (obtained from Milligen/Biosearch, California), or Sepharose (Pharmacia, Sweden). In another embodiment, the solid support can be a magnetic bead coated with streptavidin, such as Dynabeads Streptavidin (Dynal Biotech, Oslo, Norway).

In one embodiment, the solid phase support is suitable for in vivo use, i.e., it can serve as a carrier or support for administration of the test compound to a patient (e.g., TENTAGEL, Bayer, Tubingen, Germany). In a particular embodiment, the solid support is palatable and/or orally ingestable.

In some embodiments of the present invention, compounds can be attached to solid supports via linkers. Linkers can be integral and part of the solid support, or they may be nonintegral that are either synthesized on the solid support or attached thereto after synthesis. Linkers are useful not only for providing points of test compound attachment to the solid support, but also for allowing different groups of molecules to be cleaved from the solid support under different conditions, depending on the nature of the linker. For example, linkers can be, inter alia, electrophilically cleaved, nucleophilically cleaved, photocleavable, enzymatically cleaved, cleaved by metals, cleaved under reductive conditions or cleaved under oxidative conditions.

### 4.4. Library Screening

After a target nucleic acid, such as but not limited to RNA or DNA, is labeled and a test compound library is synthesized or purchased or both, the labeled target nucleic acid is used to screen the library to identify test compounds that bind to the nucleic acid. Screening comprises contacting a labeled target nucleic acid with an individual, or small group, of the components of the compound library. Preferably, the contacting occurs in an aqueous solution, and most preferably, under physiologic conditions. The aqueous solution preferably stabilizes the labeled target nucleic acid and prevents denaturation or degradation of the nucleic acid without interfering with binding of the test compounds. The aqueous solution can be similar to the solution in which a complex between the target RNA and its corresponding host cell factor is formed in vitro. For example, TK buffer, which is commonly used to form Tat protein-TAR RNA complexes in vitro, can be used in the methods of the invention as an aqueous solution to screen a library of test compounds for TAR RNA binding compounds.

The methods of the present invention for screening a library of test compounds preferably comprise contacting a test compound with a target nucleic acid in the presence of an aqueous solution, the aqueous solution comprising a buffer and a combination of salts, preferably approximating or mimicking physiologic conditions. The aqueous solution optionally further comprises non-specific nucleic acids, such as, but not limited to, DNA; yeast tRNA; salmon sperm DNA; homoribopolymers such as, but not limited to, poly IC, polyA, polyU, and polyC; and non-specific RNA. The non-specific RNA may be an unlabeled target nucleic acid having a mutation at the binding site, which renders the unlabeled nucleic acid incapable of interacting with a test compound at that site. For example, if dye-labeled TAR RNA is used to screen a library, unlabeled TAR RNA having a mutation in the uracil 23/cytosine 24 bulge region may also be present in the aqueous solution. Without being bound by any theory, the addition of unlabeled RNA that is essentially identical to the dye-labeled target RNA except for a mutation at the binding site might minimize interactions of other regions of the dye-labeled target RNA with test compounds or with the solid support and prevent false positive results.

The solution further comprises a buffer, a combination of salts, and optionally, a detergent or a surfactant. The pH of the solution typically ranges from about 5 to about 8, preferably from about 6 to about 8, most preferably from about 6.5 to about 8. A variety of buffers may be used to achieve the desired pH. Suitable buffers include, but are not limited to, Tris, Mes, Bis-Tris, Ada, Aces, Pipes, Mopso, Bis-Tris propane, Bes, Mops, Tes, Hepes, Dipso, Mobs, Tapso, Trizma, Heppso, Popso, TEA, Epps, Tricine, Gly-Gly, Bicine, and sodium-potassium phosphate. The buffering agent comprises from about 10 mM to about 100 mM, preferably from about 25 mM to about 75 mM, most preferably from about 40 mM to about 60 mM buffering agent. The pH of the aqeuous solution can be optimized for different screening reactions, depending on the target RNA used and the types of test compounds in the library, and therefore, the type and amount of the buffer used in the solution can vary from screen to screen. In a preferred embodiment, the aqueous solution has pH of about 7.4, which can be achieved using about 50 mM Tris buffer.

In addition to an appropriate buffer, the aqueous solution further comprises a combination of salts, from about 0 mM to about 100 mM KCl, from about 0 mM to about 1 M NaCl, and from about 0 mM to about 200 mM MgCl₂. In a preferred embodiment, the combination of salts is about 100 mM KCl, 500 mM NaCl, and 10 mM MgCl₂. Without being bound by any theory, Applicant has found that a combination of KCl, NaCl, and MgCl₂ stabilizes the target RNA such that most of the RNA is not denatured or digested over the course of the screening reaction. The optional concentration of each salt used in the aqueous solution is dependent on the particular target RNA used and can be determined using routine experimentation.

The solution optionally comprises from about 0.01% to about 0.5% (w/v) of a detergent or a surfactant. Without being bound by any theory, a small amount of detergent or surfactant in the solution might reduce non-specific binding of the target RNA to the solid support and control aggregation and increase stability of target RNA molecules. Typical detergents useful in the methods of the present invention include, but are not limited to, anionic detergents, such as salts of deoxycholic acid, 1-heptanesulfonic acid, N-laurylsarcosine, lauryl sulfate, 1-octane sulfonic acid and taurocholic acid; cationic detergents such as benzalkonium chloride, cetylpyridinium, methylbenzethonium chloride, and decamethonium bromide; zwitterionic detergents such as CHAPS, CHAPSO, alkyl betaines, alkyl amidoalkyl betaines, N-dodecyl-N,N-dimethyl-3-ammonio-1-propanesulfonate, and phosphatidylcholine; and non-ionic detergents such as n-decyl a-D-glucopyranoside, n-decyl β-D-maltopyranoside, n-dodecyl β-D-maltoside, n-octyl β-D-glucopyranoside, sorbitan esters, n-tetradecyl β-D-maltoside, octylphenoxy polyethoxyethanol (Nonidet P-40), nonylphenoxypolyethoxyethanol (NP-40), and tritons. Preferably, the detergent, if present, is a nonionic detergent. Typical surfactants useful in the methods of the present invention include, but are not limited to, ammonium lauryl sulfate, polyethylene glycols, butyl glucoside, decyl glucoside, Polysorbate 80, lauric acid, myristic acid, palmitic acid, potassium palmitate, undecanoic acid, lauryl betaine, and lauryl alcohol. More preferably, the detergent, if present, is Triton X-100 and present in an amount of about 0.1% (w/v).

Non-specific binding of a labeled target nucleic acid to test compounds can be further minimized by treating the binding reaction with one or more blocking agents. In one embodiment, the binding reactions are treated with a blocking agent, e.g., bovine serum albumin (“BSA”), before contacting with to the labeled target nucleic acid. In another embodiment, the binding reactions are treated sequentially with at least two different blocking agents. This blocking step is preferably performed at room temperature for from about 0.5 to about 3 hours. In a subsequent step, the reaction mixture is further treated with unlabeled RNA having a mutation at the binding site. This blocking step is preferably performed at about 4° C. for from about 12 hours to about 36 hours before addition of the dye-labeled target RNA. Preferably, the solution used in the one or more blocking steps is substantially similar to the aqueous solution used to screen the library with the dye-labeled target RNA, e.g., in pH and salt concentration.

Once contacted, the mixture of labeled target nucleic acid and the test compound is preferably maintained at 4° C. for from about 1 day to about 5 days, preferably from about 2 days to about 3 days with constant agitation. To identify the reactions in which binding to the labeled target nucleic acid occurred, after the incubation period, bound from free compounds are determined using any of the methods disclosed in Section 4.5 infra.

### 4.5. Separation Methods for Screening Test Compounds

After the labeled target RNA is contacted with the library of test compounds immobilized on beads, the beads must then be separated from the unbound target RNA in the liquid phase. This can be accomplished by any number of physical means; e.g., sedimentation, centrifugation. Thereafter, a number of methods can be used to separate the library beads that are complexed with the labeled target RNA from uncomplexed beads in order to isolate the test compound on the bead. Alternatively, mass spectroscopy and NMR spectroscopy can be used to simultaneously identify and separate beads complexed to the labeled target RNA from uncomplexed beads.

### 4.5.1. Flow Cytometry

In a preferred embodiment, the complexed and non-complexed target nucleic acids are separated by flow cytometry methods. Flow cytometers for sorting and examining biological cells are well known in the art; this technology can be applied to separate the labeled library beads from unlabeled beads. Known flow cytometers are described, for example, in U.S. Pat. Nos. 4,347,935; 5,464,581; 5,483,469; 5,602,039; 5,643,796; and 6,211,477; the entire contents of which are incorporated by reference herein. Other known flow cytometers are the FACS Vantage™ system manufactured by Becton Dickinson and Company, and the COPAS™ system manufactured by Union Biometrica.

A flow cytometer typically includes a sample reservoir for receiving a biological sample. The biological sample contains particles (hereinafter referred to as “beads”) that are to be analyzed and sorted by the flow cytometer. Beads are transported from the sample reservoir at high speed (>100 beads/second) to a flow cell in a stream of liquid “sheath fluid. High-frequency vibrations of a nozzle that directs the stream to the flow cell causes the stream to partition and form ordered droplets, with each droplet containing a single bead. Physical properties of beads can be measured as they intersect a laser beam within the cytometer flow cell. As beads move one by one through the interrogation point, they cause the laser light to scatter and fluorescent molecules on the labeled beads (i.e., beads complexed with labeled target RNA) become excited. Alternatively, if the target nucleic acid is labeled with an inorganic nanoparticle, the beads complexed with bound target nucleic acid can be distinguished not only by unique fluorescent properties but also on the basis of spectrometric properties (e.g. including but not limited to increased optical density due to the reduction of Ag⁺ ions in the presence of gold nanoparticles (see, e.g., Taton et al. Science 2000, 289: 1757-1760)).

An appropriate detection system consisting of photomultiplier tubes, photodiodes or other devices for measuring light are focused onto the interrogation point where the properties are measured. In so doing, information regarding particle size (light scatter) and complex formation (fluorescence intensity) is obtained. Particles with the desired physical properties are then sorted by a variety of physical means. In one embodiment, the beads are sorted by an electrostatic method. To sort beads by an electrostatic method, the droplets containing the beads with the desired physical properties are electrically charged and deflected from the trajectory of uncharged droplets as they pass through an electrostatic field formed by two deflection plates held constant at a high electrical potential difference. In another embodiment, the beads are sorted by an air-diverting method. To sort beads by an air-diverting method, the droplets containing the beads with the desired physical properties are deflected from their trajectory by a focused stream of forced air. Both of these embodiments cause the trajectory of beads with the desired physical properties to become changed, thereby sorting them from other beads. Accordingly, the beads complexed to the labeled target RNA can be collected in an appropriate collecting vessel.

Thus, in one embodiment of the present invention, the complexed and non-complexed target nucleic acids are separated by flow cytometry methods. In a preferred embodiment, the target nucleic acid is labeled with a fluorescent label and the complexed and non-complexed target nucleic acids are separated by fluorescence activated cell sorting (“FACS”). Such methods are well known to one of skill in the art.

### 4.5.2. Affinity Chromatography

In another embodiment of the invention, the target RNA can be labeled with biotin, an antigen, or a ligand. Library beads complexed to the target RNA can be separated from uncomplexed beads using affinity techniques designed to capture the labeled moiety on the target RNA. For example, a solid support, such as but not limited to, a column or a well in a microwell plate coated with avidin/streptavidin, an antibody to the antigen, or a receptor for the ligand can be used to capture or immobilize the labeled beads. Complexed RNA may or may not be irreversibly bound to the bead by a further transformation between the bound RNA and an additional moiety on the surface of the bead. Such linking methods include, but are not limited to: photochemical crosslinking between RNA and bead-bound molecules such as psoralen, thymidine or uridine derivates either present as monomers, oligomers, or as a partially complementary sequence; or chemical ligation by disulfide exchange, nitrogen mustards, bond formation between an electrophile and a nucleophile, or alkylating reagents. See, e.g., International Patent Publication WO/0146461, the contents of which are hereby incorporated by reference. The unbound library beads can be removed after the binding reaction by washing the solid phase. If the RNA is irreversibly bound to the bead, test compounds can be isolated from the bead following destruction of the bound RNA by preferably, but not limited to, enzymatic or chemical (e.g., alkaline hydrolysis) degradation. The library beads bound to the solid phase can then be eluted with any solution that disrupts the binding between the labeled target RNA and the solid phase. Such solutions include high salt solutions, low pH solutions, detergents, and chaotropic denaturants, and are well known to one of skill in the art. In another embodiment, the test compounds can be eluted from the solid phase by heat.

In one embodiment, the library of test compounds can be prepared on magnetic beads, such as Dynabeads Streptavidin (Dynal Biotech, Oslo, Norway). The magnetic bead library can then be mixed with the labeled target RNA under conditions that allow binding to occur. The separation of the beads from unbound target RNA in the liquid phase can be accomplished using a magnet. After removal of the magnetic field, the bead complexed to the labeled RNA may be separated from uncomplexed library beads via the label used on the target RNA; e.g., biotinylated target RNA can be captured by avidin/streptavidin; target RNA labeled with antigen can be captured by the appropriate antibody; target RNA labeled with ligand can be captured using the appropriate immobilized receptor. The captured library bead can then be eluted with any solution that disrupts the binding between the labeled target RNA and the immobilized surface. Such solutions include high salt solutions, low pH solutions, detergents, and chaotropic denaturants, and are well known to one of skill in the art. Complexed RNA may or may not be irreversibly bound to the bead by a further transformation between the bound RNA and an additional moiety on the surface of the bead. Such linking methods include, but are not limited to: photochemical crosslinking between RNA and bead-bound molecules such as psoralen, thymidine or uridine derivates either present as monomers, oligomers, or as a partially complementary sequence; or chemical ligation by disulfide exchange, nitrogen mustards, bond formation between an electrophile and a nucleophile, or alkylating reagents. See, e.g., International Patent Publication WO/0146461, the contents of which are hereby incorporated by reference. If the RNA is irreversibly bound to the bead, test compounds can be isolated from the bead following destruction of the bound RNA by enzymatic degradation including, but not limited to, ribonucleases A, U₂, CL₃, T₁, Phy M, B. cereus or chemical degradation including, but not limited to, piperidine-promoted backbone cleavage of abasic sites (following treatment with sodium hydroxide, hydrazine, piperidine formate, or dimethyl sulfate), or metal-assisted (e.g. nickel(II), cobalt(II), or iron(II)) oxidative cleavage.

In another embodiment, the preselected target RNA can be labeled with a heavy metal tag and incubated with the library beads to allow binding of the test compounds to the target RNA. The separation of the labeled beads from unlabeled beads can be accomplished using a magnetic field. After removal of the magnetic field, the test compound can be eluted with any solution that disrupts the binding between the preselected target RNA and the test compound. Such solutions include high salt solutions, low pH solutions, detergents, and chaotropic denaturants, and are well known to one of skill in the art. In another embodiment, the test compounds can be eluted from the solid phase by heat.

### 4.5.3. Manual Batch

In one embodiment, a manual “batch” mode is used for separating complexed beads. To explore a bead-based library within a reasonable time period, the primary screens should be operated with sufficient throughput. To do this, the target nucleic acid is labeled with a dye and then incubated with the combinatorial library. An advantage of such an assay is the fast identification of active library beads by color change. In the lower concentrations of the dye-labeled target molecule, only those library beads that bind the target molecules most tightly are detected because of higher local concentration of the dye. When washed and plated into a liquid monolayer, colored beads are easily separated from non-colored beads with the aid of a dissecting microscope. One of the problems associated with this method could be the interaction between the red dye and library substrates. Control experiments using the dye alone and dye attached to mutant RNA sequences with the libraries are performed to eliminate this possibility.

### 4.5.4. Suspension of Beads in Electric Fields

In another embodiment of the invention, library beads bound to the target RNA can be separated from unbound beads on the basis of the altered charge properties due to RNA binding. In a preferred embodiment of this technique, beads are separated from unbound nucleic acid and suspended, preferably but not only, in the presence of an electric field where the bound RNA causes the beads bound to the target RNA to migrate toward the anode, or positive, end of the field.

Beads can be preferentially suspended in solution as a colloidal suspension with the aid of detergents or surfactants. Typical detergents useful in the methods of the present invention include, but are not limited to, anionic detergents, such as salts of deoxycholic acid, 1-heptanesulfonic acid, N-laurylsarcosine, lauryl sulfate, 1-octane sulfonic acid, carboxymethylcellulose, carrageenan, and taurocholic acid; cationic detergents such as benzalkonium chloride, cetylpyridinium, methylbenzethonium chloride, and decamethonium bromide; zwitterionic detergents such as CHAPS, CHAPSO, alkyl betaines, alky amidoalkyl betaines, N-dodecyl-N,N-dimethyl-3-ammonio-1-propanesulfonate, and phosphatidylcholine; and non-ionic detergents such as n-decyl α-D-glucopyranoside, n-decyl-D-maltopyranoside, n-dodecyl-D-maltoside, n-octyl-D-glucopyranoside, sorbitan esters, n-tetradecyl-D-maltoside and tritons. Preferably, the detergent, if present, is a nonionic detergent. Typical surfactants useful in the methods of the present invention include, but are not limited to, ammonium lauryl sulfate, polyethylene glycols, butyl glucoside, decyl glucoside, Polysorbate 80, lauric acid, myristic acid, palmitic acid, potassium palmitate, undecanoic acid, lauryl betaine, and lauryl alcohol.

Complexed RNA may or may not be irreversibly bound to the bead by a further transformation between the bound RNA and an additional moiety on the surface of the bead. Such linking methods include, but are not limited to: photochemical crosslinking between RNA and bead-bound molecules such as psoralen, thymidine or uridine derivates either present as monomers, oligomers, or as a partially complementary sequence; or chemical ligation by disulfide exchange, nitrogen mustards, bond formation between an electrophile and a nucleophile, or alkylating reagents.

If the RNA is irreversibly bound to the bead, test compounds can be isolated from the bead following destruction of the bound RNA by enzymatic degradation including, but not limited to, ribonucleases A, U₂, CL₃, T₁, Phy M, B. cereus or chemical degradation including, but not limited to, piperidine-promoted backbone cleavage of abasic sites (following treatment with sodium hydroxide, hydrazine, piperidine formate, or dimethyl sulfate), or metal-assisted (e.g. nickel(II), cobalt(II), or iron(II)) oxidative cleavage.

### 4.5.5. Microwave

In another embodiment, the complexed beads are separated from uncomplexed beads by microwave. For example, as described in U.S. Pat. Nos. 6,340,568; 6,338,968; and 6,287,874 to Hefti, the disclosures of which are hereby incorporated by reference, a system which is sensitive to the unique dielectric properties of molecules and binding complexes, such as hybridization complexes formed between a nucleic acid probe and a nucleic acid target, molecular binding events, and protein/ligand complexes, can be used to analyze nucleic acids. In this system, the different hybridization complexes can be directly distinguished without the use of labels. The method involves contacting a nucleic acid probe that is electromagnetically coupled to a portion of a signal path with a sample containing a target nucleic acid. The portion of the signal path to which the nucleic acid probe is coupled typically is a continuous transmission line. A response signal is detected for a hybridization complex formed between the nucleic acid probe and the nucleic acid target. Detection may involve propagating a test signal along the signal path and then detecting a response signal formed through modulation of the test signal by the hybridization complex.

### 4.6. Methods for Identifying Test Compounds

If the library is a peptide or nucleic acid library, the sequence of the test compound on the isolated bead can be determined by direct sequencing of the peptide or nucleic acid. Such methods are well known to one of skill in the art.

### 4.6.1. Mass Spectrometry

Mass spectrometry (e.g., electrospray ionization (“ESI”) and matrix-assisted laser desorption-ionization (“MALDI”), Fourier-transform ion cyclotron resonance (“FT-ICR”)) can be used both for high-throughput screening of test compounds that bind to a target RNA and elucidating the structure of the test compound on the isolated bead.

MALDI uses a pulsed laser for desorption of the ions and a time-of-flight analyzer, and has been used for the detection of noncovalent tRNA:amino-acyl-tRNA synthetase complexes (Gruic-Sovulj et al., 1997, J. Biol. Chem. 272:32084-32091). However, covalent cross-linking between the target nucleic acid and the test compound is required for detection, since a non-covalently bound complex may dissociate during the MALDI process.

ESI mass spectrometry (“ESI-MS”) has been of greater utility for studying non-covalent molecular interactions because, [symbol] ke the MALDI process, ESI-MS generates molecular ions with little to no fragmentation (Xavier et al., 2000, Trends Biotechnol. 18(8):349-356). ESI-MS has been used to study the complexes formed by HIV Tat peptide and protein with the TAR RNA (Sannes-Lowery et al., 1997, Anal. Chem. 69:5130-5135).

Fourier-transform ion cyclotron resonance (“FT-ICR”) mass spectrometry provides high-resolution spectra, isotope-resolved precursor ion selection, and accurate mass assignments (Xavier et al., 2000, Trends Biotechnol. 18(8):349-356). FT-ICR has been used to study the interaction of aminoglycoside antibiotics with cognate and non-cognate RNAs (Hofstadler et al., 1999, Anal. Chem. 71:3436-3440; Griffey et al., 1999, Proc. Natl. Acad. Sci. USA 96:10129-10133). As true for all of the mass spectrometry methods discussed herein, FT-ICR does not require labeling of the target RNA or a test compound.

An advantage of mass spectroscopy is not only the elucidation of the structure of the test compound, but also the determination of the structure of the test compound bound to the preselected target RNA. Such information can enable the discovery of a consensus structure of a test compound that specifically binds to a preselected target RNA.

In a preferred embodiment, the structure of the test compound is determined by time of flight mass spectroscopy (“TOF-MS”). In time of flight methods of mass spectrometry, charged (ionized) molecules are produced in a vacuum and accelerated by an electric field into a time of flight tube or drift tube. The velocity to which the molecules may be accelerated is proportional to the accelerating potential, proportional to the charge of the molecule, and inversely proportional to the square of the mass of the molecule. The charged molecules travel, i.e., “drift” down the TOF tube to a detector. The time taken for the molecules to travel down the tube may be interpreted as a measure of their molecular weight. Time-of-flight mass spectrometers have been developed for all of the major ionization techniques such as, but limited to, electron impact (“EI”), infrared laser desorption (“IRLD”), plasma desorption (“PD”), fast atom bombardment (“FAB”), secondary ion mass spectrometry (“SIMS”), matrix-assisted laser desorption/ionization (“MALDI”), and electrospray ionization (“ESI”).

### 4.6.2. NMR Spectroscopy

NMR spectroscopy can be used for elucidating the structure of the test compound on the isolated bead. NMR spectroscopy is a technique for identifying binding sites in target nucleic acids by qualitatively determining changes in chemical shift, specifically from distances measured using relaxation effects. Examples of NMR that can be used for the invention include, but are not limited to, one-dimentional NMR, two-dimentional NMR, correlation spectroscopy (“COSY”), and nuclear Overhauser effect (“NOE”) spectroscopy. Such methods of structure determination of test compounds are well known to one of skill in the art.

Similar to mass spectroscopy, an advantage of NMR is the not only the elucidation of the structure of the test compound, but also the determination of the structure of the test compound bound to the preselected target RNA. Such information can enable the discovery of a consensus structure of a test compound that specifically binds to a preselected target RNA.

### 4.6.3. Edman Degradation

In an embodiment wherein the library is a peptide library or a derivative thereof, Edman degradation can be used to determine the structure of the test compound. In one embodiment, a modified Edman degradation process is used to obtain compositional tags for proteins, which is described in U.S. Pat. No. 6,277,644 to Farnsworth et al., which is hereby incorporated by reference in its entirety. The Edman degradation chemistry is separated from amino acid analysis, circumventing the serial requirement of the conventional Edman process. Multiple cycles of coupling and cleavage are performed prior to extraction and compositional analysis of amino acids. The amino acid composition information is then used to search a database of known protein or DNA sequences to identify the sample protein. An apparatus for performing this method comprises a sample holder for holding the sample, a coupling agent supplier for supplying at least one coupling agent, a cleavage agent supplier for supplying a cleavage agent, a controller for directing the sequential supply of the coupling agents, cleavage agents, and other reagents necessary for performing the modified Edman degradation reactions, and an analyzer for analyzing amino acids.

In another embodiment, the method can be automated as described in U.S. Pat. No. 5,565,171 to Dovichi et al., which is hereby incorporated by reference in its entirety. The apparatus includes a continuous capillary connected between two valves that control fluid flow in the capillary. One part of the capillary forms a reaction chamber where the sample may be immobilized for subsequent reaction with reagents supplied through the valves. Another part of the capillary passes through or terminates in the detector portion of an analyzer such as an electrophoresis apparatus, liquid chromatographic apparatus or mass spectrometer. The apparatus may form a peptide or protein sequencer for carrying out the Edman degradation reaction and analyzing the reaction product produced by the reaction. The protein or peptide sequencer includes a reaction chamber for carrying out coupling and cleavage on a peptide or protein to produce derivatized amino acid residue, a conversion chamber for carrying out conversion and producing a coverted amino acid residue and an analyzer for identifying the converted amino acid residue. The reaction chamber may be contained within one arm of a capillary and the conversion chamber is located in another arm of the capillary. An electrophoresis length of capillary is directly capillary coupled to the conversion chamber to allow electrophoresis separation of the converted amino acid residue as it leaves the conversion chamber. Identification of the converted amino acid residue takes place at one end of the electrophoresis length of the capillary.

### 4.6.4. Vibrational Spectroscopy

Vibrational spectroscopy (e.g. infrared (IR) spectroscopy or Raman spectroscopy) can be used for elucidating the structure of the test compound on the isolated bead.

Infrared spectroscopy measures the frequencies of infrared light (wavelengths from 100 to 10,000 nm) absorbed by the test compound as a result of excitation of vibrational modes according to quantum mechanical selection rules which require that absorption of light cause a change in the electric dipole moment of the molecule. The infrared spectrum of any molecule is a unique pattern of absorption wavelengths of varying intensity that can be considered as a molecular fingerprint to identify any compound.

Infrared spectra can be measured in a scanning mode by measuring the absorption of individual frequencies of light, produced by a grating which separates frequencies from a mixed-frequency infrared light source, by the test compound relative to a standard intensity (double-beam instrument) or pre-measured (‘blank’) intensity (single-beam instrument). In a preferred embodiment, infrared spectra are measured in a pulsed mode (FT-IR) where a mixed beam, produced by an interferometer, of all infrared light frequencies is passed through or reflected off the test compound. The resulting interferogram, which may or may not be added with the resulting interferograms from subsequent pulses to increase the signal strength while averaging random noise in the electronic signal, is mathematically transformed into a spectrum using Fourier Transform or Fast Fourier Transform algorithms.

Raman spectroscopy measures the difference in frequency due to absorption of infrared frequencies of scattered visible or ultraviolet light relative to the incident beam. The incident monochromatic light beam, usually a single laser frequency, is not truly absorbed by the test compound but interacts with the electric field transiently. Most of the light scattered off the sample with be unchanged (Rayleigh scattering) but a portion of the scatter light will have frequencies that are the sum or difference of the incident and molecular vibrational frequencies. The selection rules for Raman (inelastic) scattering require a change in polarizability of the molecule. While some vibrational transitions are observable in both infrared and Raman spectrometry, must are observable only with one or the other technique. The Raman spectrum of any molecule is a unique pattern of absorption wavelengths of varying intensity that can be considered as a molecular fingerprint to identify any compound.

Raman spectra are measured by submitting monochromatic light to the sample, either passed through or preferably reflected off, filtering the Rayleigh scattered light, and detecting the frequency of the Raman scattered light. An improved Raman spectrometer is described in U.S. Pat. No. 5,786,893 to Fink et al., which is hereby incorporated by reference.

Vibrational microscopy can be measured in a spatially resolved fashion to address single beads by integration of a visible microscope and spectrometer. A microscopic infrared spectrometer is described in U.S. Pat. No. 5,581,085 to Reffner et al., which is hereby incorporated by reference in its entirety. An instrument that simultaneously performs a microscopic infrared and microscopic Raman analysis on a sample is described in U.S. Pat. No. 5,841,139 to Sostek et al., which is hereby incorporated by reference in its entirety.

In one embodiment of the method, test compounds are synthesized on polystyrene beads doped with chemically modified styrene monomers such that each resulting bead has a characteristic pattern of absorption lines in the vibrational (IR or Raman) spectrum, by methods including but not limited to those described by Fenniri et al., 2000, J. Am. Chem. Soc. 123:8151-8152. Using methods of split-pool synthesis familiar to one of skill in the art, the library of compounds is prepared so that the spectroscopic pattern of the bead identifies one of the components of the test compound on the bead. Beads that have been separated according to their ability to bind target RNA can be identified by their vibrational spectrum. In one embodiment of the method, appropriate sorting and binning of the beads during synthesis then allows identification of one or more further components of the test compound on any one bead. In another embodiment of the method, partial identification of the compound on a bead is possible through use of the spectroscopic pattern of the bead with or without the aid of further sorting during synthesis, followed by partial resynthesis of the possible compounds aided by doped beads and appropriate sorting during synthesis.

In another embodiment, the IR or Raman spectra of test compounds are examined while the compound is still on a bead, preferably, or after cleavage from bead, using methods including but not limited to photochemical, acid, or heat treatment. The test compound can be identified by comparison of the IR or Raman spectral pattern to spectra previously acquired for each test compound in the combinatorial library.

### 4.7. Secondary Biological Screens

The test compounds identified in the binding assay (for convenience referred to herein as a “lead” compound) can be tested for biological activity using host cells containing or engineered to contain the target RNA element coupled to a functional readout system. For example, the lead compound can be tested in a host cell engineered to contain the target RNA element controlling the expression of a reporter gene. In this example, the lead compounds are assayed in the presence or absence of the target RNA. Alternatively, a phenotypic or physiological readout can be used to assess activity of the target RNA in the presence and absence of the lead compound.

In one embodiment, the lead compound can be tested in a host cell engineered to contain the target RNA element controlling the expression of a reporter gene, such as, but not limited to, β-galactosidase, green fluorescent protein, red fluorescent protein, luciferase, chloramphenicol acetyltransferase, alkaline phosphatase, and β-lactamase. In a preferred embodiment, a cDNA encoding the target element is fused upstream to a reporter gene wherein translation of the reporter gene is repressed upon binding of the lead compound to the target RNA. In other words, the steric hindrance caused by the binding of the lead compound to the target RNA repressed the translation of the reporter gene. This method, termed the translational repression assay procedure (“TRAP”) has been demonstrated in E. coli and S. cerevisiae (Jain & Belasco, 1996, Cell 87(1):115-25; Huang & Schreiber, 1997, Proc. Natl. Acad. Sci. USA 94:13396-13401).

In another embodiment, a phenotypic or physiological readout can be used to assess activity of the target RNA in the presence and absence of the lead compound. For example, the target RNA may be overexpressed in a cell in which the target RNA is endogenously expressed. Where the target RNA controls expression of a gene product involved in cell growth or viability, the in vivo effect of the lead compound can be assayed by measuring the cell growth or viability of the target cell. Alternatively, a reporter gene can also be fused downstream of the target RNA sequence and the effect of the lead compound on reporter gene expression can be assayed.

Alternatively, the lead compounds identified in the binding assay can be tested for biological activity using animal models for a disease, condition, or syndrome of interest. These include animals engineered to contain the target RNA element coupled to a functional readout system, such as a transgenic mouse. Animal model systems can also be used to demonstrate safety and efficacy.

Compounds displaying the desired biological activity can be considered to be lead compounds, and will be used in the design of congeners or analogs possessing useful pharmacological activity and physiological profiles. Following the identification of a lead compound, molecular modeling techniques can be employed, which have proven to be useful in conjunction with synthetic efforts, to design variants of the lead that can be more effective. These applications may include, but are not limited to, Pharmacophore Modeling (cf Lamothe, et al. 1997, J. Med. Chem. 40: 3542; Mottola et al. 1996, J. Med. Chem. 39: 285; Beusen et al. 1995, Biopolymers 36: 181; P. Fossa et al. 1998, Comput. Aided Mol. Des. 12: 361), QSAR development (cf. Siddiqui et al. 1999, J. Med. Chem. 42: 4122; Barreca et al. 1999 Bioorg. Med. Chem. 7: 2283; Kroemer et al. 1995, J. Med. Chem. 38: 4917; Schaal et al. 2001, J. Med. Chem. 44: 155; Buolamwini & Assefa 2002, J. Mol. Chem. 45: 84), Virtual docking and screening/scoring (cf Anzini et al. 2001, J. Med. Chem. 44: 1134; Faaland et al. 2000, Biochem. Cell. Biol. 78: 415; Silvestri et al. 2000, Bioorg. Med. Chem. 8: 2305; J. Lee et al. 2001, Bioorg. Med. Chem. 9: 19), and Structure Prediction using RNA structural programs including, but not limited to mFold (as described by Zuker et al. Algorithms and Thermodynamics for RNA Secondary Structure Prediction: A Practical Guide in RNA Biochemistry and Biotechnology pp. 11-43, J. Barciszewski & B. F. C. Clark, eds. (NATO ASI Series, Kluwer Academic Publishers, 1999) and Mathews et al. 1999 J. Mol. Biol. 288: 911-940); RNAmotif (Macke et al. 2001, Nucleic Acids Res. 29: 4724-4735; and the Vienna RNA package (Hofacker et al. 1994, Monatsh. Chem. 125: 167-188).

Further examples of the application of such techniques can be found in several review articles, such as Rotivinen et al., 1988, Acta Pharmaceutical Fennica 97:159-166; Ripka, 1998, New Scientist 54-57; McKinaly & Rossmann, 1989, Annu. Rev. Pharmacol. Toxiciol. 29:111-122; Perry & Davies, QSAR: Quantitative Structure-Activity Relationships in Drug Design pp. 189-193 (Alan R. Liss, Inc. 1989); Lewis & Dean, 1989, Proc. R. Soc. Lond. 236:125-140 and 141-162; Askew et al., 1989, J. Am. Chem. Soc. 111: 1082-1090. Molecular modeling tools employed may include those from Tripos, Inc., St. Louis, Mo. (e.g., Sybyl/UNITY, CONCORD, DiverseSolutions), Accelerys, San Diego, Calif. (e.g., Catalyst, Wisconsin Package {BLAST, etc.}), Schrodinger, Portland, Oreg. (e.g., QikProp, QikFit, Jaguar) or other such vendors as BioDesign, Inc. (Pasadena, Calif.), Allelix, Inc. (Mississauga, Ontario, Canada), and Hypercube, Inc. (Cambridge, Ontario, Canada), and may include privately designed and/or “academic” software (e.g. RNAMotif, mFOLD). These application suites and programs include tools for the atomistic construction and analysis of structural models for drug-like molecules, proteins, and DNA or RNA and their potential interactions. They also provide for the calculation of important physical properties, such as solubility estimates, permeability metrics, and empirical measures of molecular “druggability” (e.g., Lipinski “Rule of 5” as described by Lipinski et al. 1997, Adv. Drug Delivery Rev. 23: 3-25). Most importantly, they provide appropriate metrics and statistical modeling power (such as the patented CoMFA technology in Sybyl as described in U.S. Pat. Nos. 6,240,374 and 6,185,506) to develop Quantitative Structural Activity Relationships (QSARs) which are used to guide the synthesis of more efficacious clinical development candidates while improving desirable physical properties, as determined by results from the aforementioned secondary screening protocols.

### 4.8. Use of Identified Compounds that Bind RNA to Treat/Prevent Disease

Biologically active compounds identified using the methods of the invention or a pharmaceutically acceptable salt thereof can be administered to a patient, preferably a mammal, more preferably a human, suffering from a disease whose progression is associated with a target RNA:host cell factor interaction in vivo. In certain embodiments, such compounds or a pharmaceutically acceptable salt thereof is administered to a patient, preferably a mammal, more preferably a human, as a preventative measure against a disease associated with an RNA:host cell factor interaction in vivo.

In one embodiment, “treatment” or “treating” refers to an amelioration of a disease, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease, either physically, e.g., stabilization of a discernible symptom, physiologically, e.g., stabilization of a physical parameter, or both. In yet another embodiment, “treatment” or “treating” refers to delaying the onset of a disease.

In certain embodiments, the compound or a pharmaceutically acceptable salt thereof is administered to a patient, preferably a mammal, more preferably a human, as a preventative measure against a disease associated with an RNA:host cell factor interaction in vivo. As used herein, “prevention” or “preventing” refers to a reduction of the risk of acquiring a disease. In one embodiment, the compound or a pharmaceutically acceptable salt thereof is administered as a preventative measure to a patient. According to this embodiment, the patient can have a genetic predisposition to a disease, such as a family history of the disease, or a non-genetic predisposition to the disease. Accordingly, the compound and pharmaceutically acceptable salts thereof can be used for the treatment of one manifestation of a disease and prevention of another.

When administered to a patient, the compound or a pharmaceutically acceptable salt thereof is preferably administered as component of a composition that optionally comprises a pharmaceutically acceptable vehicle. The composition can be administered orally, or by any other convenient route, for example, by infusion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer the compound and pharmaceutically acceptable salts thereof.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of the compound or a pharmaceutically acceptable salt thereof into the bloodstream.

In specific embodiments, it may be desirable to administer the compound or a pharmaceutically acceptable salt thereof locally This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In certain embodiments, it may be desirable to introduce the compound or a pharmaceutically acceptable salt thereof into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound and pharmaceutically acceptable salts thereof can be formulated as a suppository, with traditional binders and vehicles such as triglycerides.

In another embodiment, the compound and pharmaceutically acceptable salts thereof can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compound and pharmaceutically acceptable salts thereof can be delivered in a controlled release system (see, e.g., Goodson, in Medical Applications of Controlled Release, supra, vol. 2, pp. 115-138 (1984)). Other controlled-release systems discussed in the review by Langer, 1990, Science 249:1527-1533) may be used. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of a target RNA of the compound or a pharmaceutically acceptable salt thereof, thus requiring only a fraction of the systemic dose.

Compositions comprising the compound or a pharmaceutically acceptable salt thereof (“compound compositions”) can additionally comprise a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, mammals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Compound compositions, if desired, can also contain minor amounts of wetting or emulsifying agents, or pH buffering agents.

Compound compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in Remington's Pharmaceutical Sciences, Alfonso R. Gennaro, ed., Mack Publishing Co. Easton, Pa., 19th ed., 1995, pp. 1447 to 1676, incorporated herein by reference.

In a preferred embodiment, the compound or a pharmaceutically acceptable salt thereof is formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, and the like. Such vehicles are preferably of pharmaceutical grade. Typically, compositions for intravenous administration comprise sterile isotonic aqueous buffer. Where necessary, the compositions may also include a solubilizing agent.

In another embodiment, the compound or a pharmaceutically acceptable salt thereof can be formulated for intravenous administration. Compositions for intravenous administration may optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the compound or a pharmaceutically acceptable salt thereof is to be administered by infusion, it can be dispensed, for example, with an infusion bottle containing sterile pharmaceutical grade water or saline. Where the compound or a pharmaceutically acceptable salt thereof is administered by injection, an ampoule of sterile water for injection or saline can be provided so that the ingredients may be mixed prior to administration.

The amount of a compound or a pharmaceutically acceptable salt thereof that will be effective in the treatment of a particular disease will depend on the nature of the disease, and can be determined by standard clinical techniques. In addition, in vitro or in vivo assays may optionally be employed to help identify optimal dosage ranges. The precise dose to be employed will also depend on the route of administration, and the seriousness of the disease, and should be decided according to the judgment of the practitioner and each patient's circumstances. However, suitable dosage ranges for oral administration are generally about 0.001 milligram to about 200 milligrams of a compound or a pharmaceutically acceptable salt thereof per kilogram body weight per day. In specific preferred embodiments of the invention, the oral dose is about 0.01 milligram to about 100 milligrams per kilogram body weight per day, more preferably about 0.1 milligram to about 75 milligrams per kilogram body weight per day, more preferably about 0.5 milligram to 5 milligrams per kilogram body weight per day. The dosage amounts described herein refer to total amounts administered; that is, if more than one compound is administered, or if a compound is administered with a therapeutic agent, then the preferred dosages correspond to the total amount administered. Oral compositions preferably contain about 10% to about 95% active ingredient by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. Suitable dosage ranges for intranasal administration are generally about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day. Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art.

The compound and pharmaceutically acceptable salts thereof are preferably assayed in vitro and in vivo, for the desired therapeutic or prophylactic activity, prior to use in humans. For example, in vitro assays can be used to determine whether it is preferable to administer the compound, a pharmaceutically acceptable salt thereof, and/or another therapeutic agent. Animal model systems can be used to demonstrate safety and efficacy.

A variety of compounds can be used for treating or preventing diseases in mammals. Types of compounds include, but are not limited to, peptides, peptide analogs including peptides comprising non-natural amino acids, e.g., D-amino acids, phosphorous analogs of amino acids, such as α-amino phosphonic acids and c-amino phosphinic acids, or amino acids having non-peptide linkages, nucleic acids, nucleic acid analogs such as phosphorothioates or peptide nucleic acids (“PNAs”), hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose.

### 5. EXAMPLE

### Therapeutic Targets

The therapeutic targets presented herein are by way of example, and the present invention is not to be limited by the targets described herein. The therapeutic targets presented herein as DNA sequences are understood by one of skill in the art that the sequences can be converted to RNA sequences.

### 5.1. Tumor Necrosis Factor Alpha (“TNF-α”)

GenBank Accession # X01394:

(SEQ ID NO: 6)

| | |
|---|---|
| 1 | gcagaggacc agctaagagg gagagaagca actacagacc |
| | ccccctgaaa acaaccctca |
| 61 | gacgccacat cccctgacaa gctgccaggc aggttctctt |
| | cctctcacat actgacccac |
| 121 | ggctccaccc tctctcccct ggaaaggaca ccatgagcac |
| | tgaaagcatg atccgggacg |
| 181 | tggagctggc cgaggaggcg ctccccaaga agacaggggg |
| | gccccagggc tccaggcggt |
| 241 | gcttgttcct cagcctcttc tccttcctga tcgtggcagg |
| | cgccaccacg ctcttctgcc |
| 301 | tgctgcactt tggagtgatc ggcccccaga gggaagagtt |
| | ccccagggac ctctctctaa |
| 361 | tcagccctct ggcccaggca gtcagatcat cttctcgaac |
| | cccgagtgac aagcctgtag |
| 421 | cccatgttgt agcaaaccct caagctgagg ggcagctcca |
| | gtggctgaac cgccgggcca |
| 481 | atgccctcct ggccaatggc gtggagctga gagataacca |
| | gctggtggtg ccatcagagg |
| 541 | gcctgtacct catctactcc caggtcctct tcaagggcca |
| | aggctgcccc tccacccatg |
| 601 | tgctcctcac ccacaccatc agccgcatcg ccgtctccta |
| | ccagaccaag gtcaacctcc |
| 661 | tctctgccat caagagcccc tgccagaggg agaccccaga |
| | gggggctgag gccaagccct |
| 721 | ggtatgagcc catctatctg ggaggggtct tccagctgga |
| | gaagggtgac cgactcagcg |
| 781 | ctgagatcaa tcggcccgac tatctcgact ttgccgagtc |
| | tgggcaggtc tactttggga |
| 841 | tcattgccct gtgaggagga cgaacatcca accttcccaa |
| | acgcctcccc tgccccaatc |
| 901 | cctttattac cccctccttc agacaccctc aacctcttct |
| | ggctcaaaaa gagaattggg |
| 961 | ggcttagggt cggaacccaa gcttagaact ttaagcaaca |
| | agaccaccac ttcgaaacct |
| 1021 | gggattcagg aatgtgtggc ctgcacagtg aattgctggc |
| | aaccactaag aattcaaact |
| 1081 | ggggcctcca gaactcactg gggcctacag ctttgatccc |
| | tgacatctgg aatctggaga |
| 1141 | ccagggagcc tttggttctg gccagaatgc tgcaggactt |
| | gagaagacct cacctagaaa |
| 1201 | ttgacacaag tggaccttag gccttcctct ctccagatgt |
| | ttccagactt ccttgagaca |
| 1261 | cggagcccag ccctccccat ggagccagct ccctctattt |
| | atgtttgcac ttgtgattat |
| 1321 | ttattattta tttattattt atttatttac agatgaatgt |
| | atttatttgg gagaccgggg |
| 1381 | tatcctgggg gacccaatgt aggagctgcc ttggctcaga |
| | catgttttcc gtgaaaacgg |
| 1441 | agctgaacaa taggctgttc ccatgtagcc ccctggcctc |
| | tgtgccttct tttgattatg |
| 1501 | ttttttaaaa tatttatctg attaagttgt ctaaacaatg |
| | ctgatttggt gaccaactgt |
| 1561 | cactcattgc tgagcctctg ctccccaggg gagttgtgtc |
| | tgtaatcgcc ctactattca |
| 1621 | gtggcgagaa ataaagtttg ctt |

General Target Regions:
(1) 5′ Untranslated Region—nts 1-152
(2) 3′ Untranslated Region—nts 852-1643
   Initial Specific Target Motif:
Group I AU-Rich Element (ARE) Cluster in 3′ untranslated region 5′ AUUUAUUUAUUUAUUUAUUUA 3′ (SEQ ID NO: 1)

### 5.2. Granulocyte-Macrophage Colony Stimulating Factor (“GM-CSF”)

GenBank Accession # NM_{—}000758:

(SEQ ID NO: 7)

| | |
|---|---|
| 1 | gctggaggat gtggctgcag agcctgctgc tcttgggcac |
| | tgtggcctgc agcatctctg |
| 61 | cacccgcccg ctcgcccagc cccagcacgc agccctggga |
| | gcatgtgaat gccatccagg |
| 121 | aggcccggcg tctcctgaac ctgagtagag acactgctgc |
| | tgagatgaat gaaacagtag |
| 181 | aagtcatctc agaaatgttt gacctccagg agccgacctg |
| | cctacagacc cgcctggagc |
| 241 | tgtacaagca gggcctgcgg ggcagcctca ccaagctcaa |
| | gggccccttg accatgatgg |
| 301 | ccagccacta caagcagcac tgccctccaa ccccggaaac |
| | ttcctgtgca acccagacta |
| 361 | tcacctttga aagtttcaaa gagaacctga aggactttct |
| | gcttgtcatc ccctttgact |
| 421 | gctgggagcc agtccaggag tgagaccggc cagatgaggc |
| | tggccaagcc ggggagctgc |
| 481 | tctctcatga aacaagagct agaaactcag gatggtcatc |
| | ttggagggac caaggggtgg |
| 541 | gccacagcca tggtgggagt ggcctggacc tgccctgggc |
| | cacactgacc ctgatacagg |
| 601 | catggcagaa gaatgggaat attttatact gacagaaatc |
| | agtaatattt atatatttat |
| 661 | atttttaaaa tatttattta tttatttatt taagttcata |
| | ttccatattt attcaagatg |
| 721 | ttttaccgta ataattatta ttaaaaatat gcttct |

GenBank Accession # XM_{—}003751:

(SEQ ID NO: 8)

| | |
|---|---|
| 1 | tctggaggat gtggctgcag agcctgctgc tcttgggcac |
| | tgtggcctgc agcatctctg |
| 61 | cacccgcccg ctcgcccagc cccagcacgc agccctggga |
| | gcatgtgaat gccatccagg |
| 121 | aggcccggcg tctcctgaac ctgagtagag acactgctgc |
| | tgagatgaat gaaacagtag |
| 181 | aagtcatctc agaaatgttt gacctccagg agccgacctg |
| | cctacagacc cgcctggagc |
| 241 | tgtacaagca gggcctgcgg ggcagcctca ccaagctcaa |
| | gggccccttg accatgatgg |
| 301 | ccagccacta caagcagcac tgccctccaa ccccggaaac |
| | ttcctgtgca acccagacta |
| 361 | tcacctttga aagtttcaaa gagaacctga aggactttct |
| | gcttgtcatc ccctttgact |
| 421 | gctgggagcc agtccaggag tgagaccggc cagatgaggc |
| | tggccaagcc ggggagctgc |
| 481 | tctctcatga aacaagagct agaaactcag gatggtcatc |
| | ttggagggac caaggggtgg |
| 541 | gccacagcca tggtgggagt ggcctggacc tgccctgggc |
| | cacactgacc ctgatacagg |
| 601 | catggcagaa gaatgggaat attttatact gacagaaatc |
| | agtaatattt atatatttat |
| 661 | atttttaaaa tatttattta tttatttatt taagttcata |
| | ttccatattt attcaagatg |
| 721 | ttttaccgta ataattatta ttaaaaatat gcttct |

General Target Regions:
(1) 5′ Untranslated Region—nts 1-32
(2) 3′ Untranslated Region—nts 468-789
   Initial Specific Target Motif:

Group I AU-Rich Element (ARE) Cluster in 3′ untranslated region

| | |
|---|---|
| 5′ AUUUAUUUAUUUAUUUAUUUA 3′ | (SEQ ID NO: 1) |

### 5.3. Interleukin 2 (“IL-2”)

GenBank Accession # U25676:

(SEQ ID NO: 9)

| | |
|---|---|
| 1 | atcactctct ttaatcacta ctcacattaa cctcaactcc |
| | tgccacaatg tacaggatgc |
| 61 | aactcctgtc ttgcattgca ctaattcttg cacttgtcac |
| | aaacagtgca cctacttcaa |
| 121 | gttcgacaaa gaaaacaaag aaaacacagc tacaactgga |
| | gcatttactg ctggatttac |
| 181 | agatgatttt gaatggaatt aataattaca agaatcccaa |
| | actcaccagg atgctcacat |
| 241 | ttaagtttta catgcccaag aaggccacag aactgaaaca |
| | gcttcagtgt ctagaagaag |
| 301 | aactcaaacc tctggaggaa gtgctgaatt tagctcaaag |
| | caaaaacttt cacttaagac |
| 361 | ccagggactt aatcagcaat atcaacgtaa tagttctgga |
| | actaaaggga tctgaaacaa |
| 421 | cattcatgtg tgaatatgca gatgagacag caaccattgt |
| | agaatttctg aacagatgga |
| 481 | ttaccttttg tcaaagcatc atctcaacac taacttgata |
| | attaagtgct tcccacttaa |
| 541 | aacatatcag gccttctatt tatttattta aatatttaaa |
| | ttttatattt attgttgaat |
| 601 | gtatggttgc tacctattgt aactattatt cttaatctta |
| | aaactataaa tatggatctt |
| 661 | ttatgattct ttttgtaagc cctaggggct ctaaaatggt |
| | ttaccttatt tatcccaaaa |
| 721 | atatttatta ttatgttgaa tgttaaatat agtatctatg |
| | tagattggtt agtaaaacta |
| 781 | tttaataaat ttgataaata taaaaaaaaa aaacaaaaaa |
| | aaaaa |

General Target Regions:
(1) 5′ Untranslated Region—nts 1-47
(2) 3′ Untranslated Region—nts 519-825
   Initial Specific Target Motifs:

Group III AU-Rich Element (ARE) Cluster in 3′ untranslated region

| | |
|---|---|
| 5′ NAUUUAUUUAUUUAN 3′ | (SEQ ID NO: 10) |

### 5.4. Interleukin 6 (“IL-6”)

GenBank Accession # NM_{—}000600:

(SEQ ID NO: 11)

| | |
|---|---|
| 1 | ttctgccctc gagcccaccg ggaacgaaag agaagctcta |
| | tctcgcctcc aggagcccag |
| 61 | ctatgaactc cttctccaca agcgccttcg gtccagttgc |
| | cttctccctg gggctgctcc |
| 121 | tggtgttgcc tgctgccttc cctgccccag tacccccagg |
| | agaagattcc aaagatgtag |
| 181 | ccgccccaca cagacagcca ctcacctctt cagaacgaat |
| | tgacaaacaa attcggtaca |
| 241 | tcctcgacgg catctcagcc ctgagaaagg agacatgtaa |
| | caagagtaac atgtgtgaaa |
| 301 | gcagcaaaga ggcactggca gaaaacaacc tgaaccttcc |
| | aaagatggct gaaaaagatg |
| 361 | gatgcttcca atctggattc aatgaggaga cttgcctggt |
| | gaaaatcatc actggtcttt |
| 421 | tggagtttga ggtataccta gagtacctcc agaacagatt |
| | tgagagtagt gaggaacaag |
| 481 | ccagagctgt gcagatgagt acaaaagtcc tgatccagtt |
| | cctgcagaaa aaggcaaaga |
| 541 | atctagatgc aataaccacc cctgacccaa ccacaaatgc |
| | cagcctgctg acgaagctgc |
| 601 | aggcacagaa ccagtggctg caggacatga caactcatct |
| | cattctgcgc agctttaagg |
| 661 | agttcctgca gtccagcctg agggctcttc ggcaaatgta |
| | gcatgggcac ctcagattgt |
| 721 | tgttgttaat gggcattcct tcttctggtc agaaacctgt |
| | ccactgggca cagaacttat |
| 781 | gttgttctct atggagaact aaaagtatga gcgttaggac |
| | actattttaa ttatttttaa |
| 841 | tttattaata tttaaatatg tgaagctgag ttaatttatg |
| | taagtcatat ttatattttt |
| 901 | aagaagtacc acttgaaaca ttttatgtat tagttttgaa |
| | ataataatgg aaagtggcta |
| 961 | tgcagtttga atatcctttg tttcagagcc agatcatttc |
| | ttggaaagtg taggcttacc |
| 1021 | tcaaataaat ggctaactta tacatatttt taaagaaata |
| | tttatattgt atttatataa |
| 1081 | tgtataaatg gtttttatac caataaatgg cattttaaaa |
| | aattc |

General Target Regions:
(1) 5′ Untranslated Region—nts 1-62
(2) 3′ Untranslated Region—nts 699-1125
   Initial Specific Target Motifs:

Group III AU-Rich Element (ARE) Cluster in 3′ untranslated region

| | |
|---|---|
| 5′ NAUUUAUUUAUUUAN 3′ | (SEQ ID NO: 10) |

### 5.5. Vascular Endothelial Growth Factor (“VEGF”)

GenBank Accession # AF022375:

(SEQ ID NO: 12)

| | |
|---|---|
| 1 | aagagctcca gagagaagtc gaggaagaga gagacggggt |
| | cagagagagc gcgcgggcgt |
| 61 | gcgagcagcg aaagcgacag gggcaaagtg agtgacctgc |
| | ttttgggggt gaccgccgga |
| 121 | gcgcggcgtg agccctcccc cttgggatcc cgcagctgac |
| | cagtcgcgct gacggacaga |
| 181 | cagacagaca ccgcccccag ccccagttac cacctcctcc |
| | ccggccggcg gcggacagtg |
| 241 | gacgcggcgg cgagccgcgg gcaggggccg gagcccgccc |
| | ccggaggcgg ggtggagggg |
| 301 | gtcggagctc gcggcgtcgc actgaaactt ttcgtccaac |
| | ttctgggctg ttctcgcttc |
| 361 | ggaggagccg tggtccgcgc gggggaagcc gagccgagcg |
| | gagccgcgag aagtgctagc |
| 421 | tcgggccggg aggagccgca gccggaggag ggggaggagg |
| | aagaagagaa ggaagaggag |
| 481 | agggggccgc agtggcgact cggcgctcgg aagccgggct |
| | catggacggg tgaggcggcg |
| 541 | gtgtgcgcag acagtgctcc agcgcgcgcg ctccccagcc |
| | ctggcccggc ctcgggccgg |
| 601 | gaggaagagt agctcgccga ggcgccgagg agagcgggcc |
| | gccccacagc ccgagccgga |
| 661 | gagggacgcg agccgcgcgc cccggtcggg cctccgaaac |
| | catgaacttt ctgctgtctt |
| 721 | gggtgcattg gagccttgcc ttgctgctct acctccacca |
| | tgccaagtgg tcccaggctg |
| 781 | cacccatggc agaaggagga gggcagaatc atcacgaagt |
| | ggtgaagttc atggatgtct |
| 841 | atcagcgcag ctactgccat ccaatcgaga ccctggtgga |
| | catcttccag gagtaccctg |
| 901 | atgagatcga gtacatcttc aagccatcct gtgtgcccct |
| | gatgcgatgc gggggctgct |
| 961 | ccaatgacga gggcctggag tgtgtgccca ctgaggagtc |
| | caacatcacc atgcagatta |
| 1021 | tgcggatcaa acctcaccaa ggccagcaca taggagagat |
| | gagcttccta cagcacaaca |
| 1081 | aatgtgaatg cagaccaaag aaagatagag caagacaaga |
| | aaatccctgt gggccttgct |
| 1141 | cagagcggag aaagcatttg tttgtacaag atccgcagac |
| | gtgtaaatgt tcctgcaaaa |
| 1201 | acacacactc gcgttgcaag gcgaggcagc ttgagttaaa |
| | cgaacgtact tgcagatgtg |
| 1261 | acaagccgag gcggtgagcc gggcaggagg aaggagcctc |
| | cctcagggtt tcgggaacca |
| 1321 | gatctctctc caggaaagac tgatacagaa cgatcgatac |
| | agaaaccacg ctgccgccac |
| 1381 | cacaccatca ccatcgacag aacagtcctt aatccagaaa |
| | cctgaaatga aggaagagga |
| 1441 | gactctgcgc agagcacttt gggtccggag ggcgagactc |
| | cggcggaagc attcccgggc |
| 1501 | gggtgaccca gcacggtccc tcttggaatt ggattcgcca |
| | ttttattttt cttgctgcta |
| 1561 | aatcaccgag cccggaagat tagagagttt tatttctggg |
| | attcctgtag acacacccac |
| 1621 | ccacatacat acatttatat atatatatat tatatatata |
| | taaaaataaa tatctctatt |
| 1681 | ttatatatat aaaatatata tattcttttt ttaaattaac |
| | agtgctaatg ttattggtgt |
| 1741 | cttcactgga tgtatttgac tgctgtggac ttgagttggg |
| | aggggaatgt tcccactcag |
| 1801 | atcctgacag ggaagaggag gagatgagag actctggcat |
| | gatctttttt ttgtcccact |
| 1861 | tggtggggcc agggtcctct cccctgccca agaatgtgca |
| | aggccagggc atgggggcaa |
| 1921 | atatgaccca gttttgggaa caccgacaaa cccagccctg |
| | gcgctgagcc tctctacccc |
| 1981 | aggtcagacg gacagaaaga caaatcacag gttccgggat |
| | gaggacaccg gctctgacca |
| 2041 | ggagtttggg gagcttcagg acattgctgt gctttgggga |
| | ttccctccac atgctgcacg |
| 2101 | cgcatctcgc ccccaggggc actgcctgga agattcagga |
| | gcctgggcgg ccttcgctta |
| 2161 | ctctcacctg cttctgagtt gcccaggagg ccactggcag |
| | atgtcccggc gaagagaaga |
| 2221 | gacacattgt tggaagaagc agcccatgac agcgcccctt |
| | cctgggactc gccctcatcc |
| 2281 | tcttcctgct ccccttcctg gggtgcagcc taaaaggacc |
| | tatgtcctca caccattgaa |
| 2341 | accactagtt ctgtcccccc aggaaacctg gttgtgtgtg |
| | tgtgagtggt tgaccttcct |
| 2401 | ccatcccctg gtccttccct tcccttcccg aggcacagag |
| | agacagggca ggatccacgt |
| 2461 | gcccattgtg gaggcagaga aaagagaaag tgttttatat |
| | acggtactta tttaatatcc |
| 2521 | ctttttaatt agaaattaga acagttaatt taattaaaga |
| | gtagggtttt ttttcagtat |
| 2581 | tcttggttaa tatttaattt caactattta tgagatgtat |
| | cttttgctct ctcttgctct |
| 2641 | cttatttgta ccggtttttg tatataaaat tcatgtttcc |
| | aatctctctc tccctgatcg |
| 2701 | gtgacagtca ctagcttatc ttgaacagat atttaatttt |
| | gctaacactc agctctgccc |
| 2761 | tccccgatcc cctggctccc cagcacacat tcctttgaaa |
| | gagggtttca atatacatct |
| 2821 | acatactata tatatattgg gcaacttgta tttgtgtgta |
| | tatatatata tatatgttta |
| 2881 | tgtatatatg tgatcctgaa aaaataaaca tcgctattct |
| | gttttttata tgttcaaacc |
| 2941 | aaacaagaaa aaatagagaa ttctacatac taaatctctc |
| | tcctttttta attttaatat |
| 3001 | ttgttatcat ttatttattg gtgctactgt ttatccgtaa |
| | taattgtggg gaaaagatat |
| 3061 | taacatcacg tctttgtctc tagtgcagtt tttcgagata |
| | ttccgtagta catatttatt |
| 3121 | tttaaacaac gacaaagaaa tacagatata tcttaaaaaa |
| | aaaaaa |

General Target Regions:
(1) 5′ Untranslated Region—nts 1-701
(2) 3′ Untranslated Region—nts 1275-3166
   Initial Specific Target Motifs:

(1) Internal Ribosome Entry Site (IRES) in 5′ untranslated region nts 513-704

(SEQ ID NO: 13)

5′CCGGGCUCAUGGACGGGUGAGGCGGCGGUGUGCGCAGACAGUG
CUCCAGCGCGCGCGCUCCCCAGCCCUGGCCCGGCCUCGGGCCGGG
AGGAAGAGUAGCUCGCCGAGGCGCCGAGGAGAGCGGGCCGCCCC
ACAGCCCGAGCCGGAGAGGGACGCGACCCGCGCGCCCCGGUCGG
GCCUCCGAAACCAUGAACUUUCUGCUGUCUUGGGUGCAUUGGAG
CCUUGCCUUGCUGCUCUACCUCCACCAUG 3′

(2) Group III AU-Rich Element (ARE) Cluster in 3′ untranslated region

| | |
|---|---|
| 5′ NAUUUAUUUAUUUAN 3′ | (SEQ ID NO: 10) |

### 5.6. Human Immunodeficiency Virus I (“HIV-1”)

GenBank Accession # NC_{—}001802:

(SEQ ID NO: 14)

| | |
|---|---|
| 1 | ggtctctctg gttagaccag atctgagcct gggagctctc |
| | tggctaacta gggaacccac |
| 61 | tgcttaagcc tcaataaagc ttgccttgag tgcttcaagt |
| | agtgtgtgcc cgtctgttgt |
| 121 | gtgactctgg taactagaga tccctcagac ccttttagtc |
| | agtgtggaaa atctctagca |
| 181 | gtggcgcccg aacagggacc tgaaagcgaa agggaaacca |
| | gaggagctct ctcgacgcag |
| 241 | gactcggctt gctgaagcgc gcacggcaag aggcgagggg |
| | cggcgactgg tgagtacgcc |
| 301 | aaaaattttg actagcggag gctagaagga gagagatggg |
| | tgcgagagcg tcagtattaa |
| 361 | gcgggggaga attagatcga tgggaaaaaa ttcggttaag |
| | gccaggggga aagaaaaaat |
| 421 | ataaattaaa acatatagta tgggcaagca gggagctaga |
| | acgattcgca gttaatcctg |
| 481 | gcctgttaga aacatcagaa ggctgtagac aaatactggg |
| | acagctacaa ccatcccttc |
| 541 | agacaggatc agaagaactt agatcattat ataatacagt |
| | agcaaccctc tattgtgtgc |
| 601 | atcaaaggat agagataaaa gacaccaagg aagctttaga |
| | caagatagag gaagagcaaa |
| 661 | acaaaagtaa gaaaaaagca cagcaagcag cagctgacac |
| | aggacacagc aatcaggtca |
| 721 | gccaaaatta ccctatagtg cagaacatcc aggggcaaat |
| | ggtacatcag gccatatcac |
| 781 | ctagaacttt aaatgcatgg gtaaaagtag tagaagagaa |
| | ggctttcagc ccagaagtga |
| 841 | tacccatgtt ttcagcatta tcagaaggag ccaccccaca |
| | agatttaaac accatgctaa |
| 901 | acacagtggg gggacatcaa gcagccatgc aaatgttaaa |
| | agagaccatc aatgaggaag |
| 961 | ctgcagaatg ggatagagtg catccagtgc atgcagggcc |
| | tattgcacca ggccagatga |
| 1021 | gagaaccaag gggaagtgac atagcaggaa ctactagtac |
| | ccttcaggaa caaataggat |
| 1081 | ggatgacaaa taatccacct atcccagtag gagaaattta |
| | taaaagatgg ataatcctgg |
| 1141 | gattaaataa aatagtaaga atgtatagcc ctaccagcat |
| | tctggacata agacaaggac |
| 1201 | caaaggaacc ctttagagac tatgtagacc ggttctataa |
| | aactctaaga gccgagcaag |
| 1261 | cttcacagga ggtaaaaaat tggatgacag aaaccttgtt |
| | ggtccaaaat gcgaacccag |
| 1321 | attgtaagac tattttaaaa gcattgggac cagcggctac |
| | actagaagaa atgatgacag |
| 1381 | catgtcaggg agtaggagga cccggccata aggcaagagt |
| | tttggctgaa gcaatgagcc |
| 1441 | aagtaacaaa ttcagctacc ataatgatgc agagaggcaa |
| | ttttaggaac caaagaaaga |
| 1501 | ttgttaagtg tttcaattgt ggcaaagaag ggcacacagc |
| | cagaaattgc agggccccta |
| 1561 | ggaaaaaggg ctgttggaaa tgtggaaagg aaggacacca |
| | aatgaaagat tgtactgaga |
| 1621 | gacaggctaa ttttttaggg aagatctggc cttcctacaa |
| | gggaaggcca gggaattttc |
| 1681 | ttcagagcag accagagcca acagccccac cagaagagag |
| | cttcaggtct ggggtagaga |
| 1741 | caacaactcc ccctcagaag caggagccga tagacaagga |
| | actgtatcct ttaacttccc |
| 1801 | tcaggtcact ctttggcaac gacccctcgt cacaataaag |
| | ataggggggc aactaaagga |
| 1861 | agctctatta gatacaggag cagatgatac agtattagaa |
| | gaaatgagtt tgccaggaag |
| 1921 | atggaaacca aaaatgatag ggggaattgg aggttttatc |
| | aaagtaagac agtatgatca |
| 1981 | gatactcata gaaatctgtg gacataaagc tataggtaca |
| | gtattagtag gacctacacc |
| 2041 | tgtcaacata attggaagaa atctgttgac tcagattggt |
| | tgcactttaa attttcccat |
| 2101 | tagccctatt gagactgtac cagtaaaatt aaagccagga |
| | atggatggcc caaaagttaa |
| 2161 | acaatggcca ttgacagaag aaaaaataaa agcattagta |
| | gaaatttgta cagagatgga |
| 2221 | aaaggaaggg aaaatttcaa aaattgggcc tgaaaatcca |
| | tacaatactc cagtatttgc |
| 2281 | cataaagaaa aaagacagta ctaaatggag aaaattagta |
| | gatttcagag aacttaataa |
| 2341 | gagaactcaa gacttctggg aagttcaatt aggaatacca |
| | catcccgcag ggttaaaaaa |
| 2401 | gaaaaaatca gtaacagtac tggatgtggg tgatgcatat |
| | ttttcagttc ccttagatga |
| 2461 | agacttcagg aagtatactg catttaccat acctagtata |
| | aacaatgaga caccagggat |
| 2521 | tagatatcag tacaatgtgc ttccacaggg atggaaagga |
| | tcaccagcaa tattccaaag |
| 2581 | tagcatgaca aaaatcttag agccttttag aaaacaaaat |
| | ccagacatag ttatctatca |
| 2641 | atacatggat gatttgtatg taggatctga cttagaaata |
| | gggcagcata gaacaaaaat |
| 2701 | agaggagctg agacaacatc tgttgaggtg gggacttacc |
| | acaccagaca aaaaacatca |
| 2761 | gaaagaacct ccattccttt ggatgggtta tgaactccat |
| | cctgataaat ggacagtaca |
| 2821 | gcctatagtg ctgccagaaa aagacagctg gactgtcaat |
| | gacatacaga agttagtggg |
| 2881 | gaaattgaat tgggcaagtc agatttaccc agggattaaa |
| | gtaaggcaat tatgtaaact |
| 2941 | ccttagagga accaaagcac taacagaagt aataccacta |
| | acagaagaag cagagctaga |
| 3001 | actggcagaa aacagagaga ttctaaaaga accagtacat |
| | ggagtgtatt atgacccatc |
| 3061 | aaaagactta atagcagaaa tacagaagca ggggcaaggc |
| | caatggacat atcaaattta |
| 3121 | tcaagagcca tttaaaaatc tgaaaacagg aaaatatgca |
| | agaatgaggg gtgcccacac |
| 3181 | taatgatgta aaacaattaa cagaggcagt gcaaaaaata |
| | accacagaaa gcatagtaat |
| 3241 | atggggaaag actcctaaat ttaaactgcc catacaaaag |
| | gaaacatggg aaacatggtg |
| 3301 | gacagagtat tggcaagcca cctggattcc tgagtgggag |
| | tttgttaata cccctccctt |
| 3361 | agtgaaatta tggtaccagt tagagaaaga acccatagta |
| | ggagcagaaa ccttctatgt |
| 3421 | agatggggca gctaacaggg agactaaatt aggaaaagca |
| | ggatatgtta ctaatagagg |
| 3481 | aagacaaaaa gttgtcaccc taactgacac aacaaatcag |
| | aagactgagt tacaagcaat |
| 3541 | ttatctagct ttgcaggatt cgggattaga agtaaacata |
| | gtaacagact cacaatatgc |
| 3601 | attaggaatc attcaagcac aaccagatca aagtgaatca |
| | gagttagtca atcaaataat |
| 3661 | agagcagtta ataaaaaagg aaaaggtcta tctggcatgg |
| | gtaccagcac acaaaggaat |
| 3721 | tggaggaaat gaacaagtag ataaattagt cagtgctgga |
| | atcaggaaag tactattttt |
| 3781 | agatggaata gataaggccc aagatgaaca tgagaaatat |
| | cacagtaatt ggagagcaat |
| 3841 | ggctagtgat tttaacctgc cacctgtagt agcaaaagaa |
| | atagtagcca gctgtgataa |
| 3901 | atgtcagcta aaaggagaag ccatgcatgg acaagtagac |
| | tgtagtccag gaatatggca |
| 3961 | actagattgt acacatttag aaggaaaagt tatcctggta |
| | gcagttcatg tagccagtgg |
| 4021 | atatatagaa gcagaagtta ttccagcaga aacagggcag |
| | gaaacagcat attttctttt |
| 4081 | aaaattagca ggaagatggc cagtaaaaac aatacatact |
| | gacaatggca gcaatttcac |
| 4141 | cggtgctacg gttagggccg cctgttggtg ggcgggaatc |
| | aagcaggaat ttggaattcc |
| 4201 | ctacaatccc caaagtcaag gagtagtaga atctatgaat |
| | aaagaattaa agaaaattat |
| 4261 | aggacaggta agagatcagg ctgaacatct taagacagca |
| | gtacaaatgg cagtattcat |
| 4321 | ccacaatttt aaaagaaaag gggggattgg ggggtacagt |
| | gcaggggaaa gaatagtaga |
| 4381 | cataatagca acagacatac aaactaaaga attacaaaaa |
| | caaattacaa aaattcaaaa |
| 4441 | ttttcgggtt tattacaggg acagcagaaa tccactttgg |
| | aaaggaccag caaagctcct |
| 4501 | ctggaaaggt gaaggggcag tagtaataca agataatagt |
| | gacataaaag tagtgccaag |
| 4561 | aagaaaagca aagatcatta gggattatgg aaaacagatg |
| | gcaggtgatg attgtgtggc |
| 4621 | aagtagacag gatgaggatt agaacatgga aaagtttagt |
| | aaaacaccat atgtatgttt |
| 4681 | cagggaaagc taggggatgg ttttatagac atcactatga |
| | aagccctcat ccaagaataa |
| 4741 | gttcagaagt acacatccca ctaggggatg ctagattggt |
| | aataacaaca tattggggtc |
| 4801 | tgcatacagg agaaagagac tggcatttgg gtcagggagt |
| | ctccatagaa tggaggaaaa |
| 4861 | agagatatag cacacaagta gaccctgaac tagcagacca |
| | actaattcat ctgtattact |
| 4921 | ttgactgttt ttcagactct gctataagaa aggccttatt |
| | aggacacata gttagcccta |
| 4981 | ggtgtgaata tcaagcagga cataacaagg taggatctct |
| | acaatacttg gcactagcag |
| 5041 | cattaataac accaaaaaag ataaagccac ctttgcctag |
| | tgttacgaaa ctgacagagg |
| 5101 | atagatggaa caagccccag aagaccaagg gccacagagg |
| | gagccacaca atgaatggac |
| 5161 | actagagctt ttagaggagc ttaagaatga agctgttaga |
| | cattttccta ggatttggct |
| 5221 | ccatggctta gggcaacata tctatgaaac ttatggggat |
| | acttgggcag gagtggaagc |
| 5281 | cataataaga attctgcaac aactgctgtt tatccatttt |
| | cagaattggg tgtcgacata |
| 5341 | gcagaatagg cgttactcga cagaggagag caagaaatgg |
| | agccagtaga tcctagacta |
| 5401 | gagccctgga agcatccagg aagtcagcct aaaactgctt |
| | gtaccaattg ctattgtaaa |
| 5461 | aagtgttgct ttcattgcca agtttgtttc ataacaaaag |
| | ccttaggcat ctcctatggc |
| 5521 | aggaagaagc ggagacagcg acgaagagct catcagaaca |
| | gtcagactca tcaagcttct |
| 5581 | ctatcaaagc agtaagtagt acatgtaatg caacctatac |
| | caatagtagc aatagtagca |
| 5641 | ttagtagtag caataataat agcaatagtt gtgtggtcca |
| | tagtaatcat agaatatagg |
| 5701 | aaaatattaa gacaaagaaa aatagacagg ttaattgata |
| | gactaataga aagagcagaa |
| 5761 | gacagtggca atgagagtga aggagaaata tcagcacttg |
| | tggagatggg ggtggagatg |
| 5821 | gggcaccatg ctccttggga tgttgatgat ctgtagtgct |
| | acagaaaaat tgtgggtcac |
| 5881 | agtctattat ggggtacctg tgtggaagga agcaaccacc |
| | actctatttt gtgcatcaga |
| 5941 | tgctaaagca tatgatacag aggtacataa tgtttgggcc |
| | acacatgcct gtgtacccac |
| 6001 | agaccccaac ccacaagaag tagtattggt aaatgtgaca |
| | gaaaatttta acatgtggaa |
| 6061 | aaatgacatg gtagaacaga tgcatgagga tataatcagt |
| | ttatgggatc aaagcctaaa |
| 6121 | gccatgtgta aaattaaccc cactctgtgt tagtttaaag |
| | tgcactgatt tgaagaatga |
| 6181 | tactaatacc aatagtagta gcgggagaat gataatggag |
| | aaaggagaga taaaaaactg |
| 6241 | ctctttcaat atcagcacaa gcataagagg taaggtgcag |
| | aaagaatatg cattttttta |
| 6301 | taaacttgat ataataccaa tagataatga tactaccagc |
| | tataagttga caagttgtaa |
| 6361 | cacctcagtc attacacagg cctgtccaaa ggtatccttt |
| | gagccaattc ccatacatta |
| 6421 | ttgtgccccg gctggttttg cgattctaaa atgtaataat |
| | aagacgttca atggaacagg |
| 6481 | accatgtaca aatgtcagca cagtacaatg tacacatgga |
| | attaggccag tagtatcaac |
| 6541 | tcaactgctg ttaaatggca gtctagcaga agaagaggta |
| | gtaattagat ctgtcaattt |
| 6601 | cacggacaat gctaaaacca taatagtaca gctgaacaca |
| | tctgtagaaa ttaattgtac |
| 6661 | aagacccaac aacaatacaa gaaaaagaat ccgtatccag |
| | agaggaccag ggagagcatt |
| 6721 | tgttacaata ggaaaaatag gaaatatgag acaagcacat |
| | tgtaacatta gtagagcaaa |
| 6781 | atggaataac actttaaaac agatagctag caaattaaga |
| | gaacaatttg gaaataataa |
| 6841 | aacaataatc tttaagcaat cctcaggagg ggacccagaa |
| | attgtaacgc acagttttaa |
| 6901 | ttgtggaggg gaatttttct actgtaattc aacacaactg |
| | tttaatagta cttggtttaa |
| 6961 | tagtacttgg agtactgaag ggtcaaataa cactgaagga |
| | agtgacacaa tcaccctccc |
| 7021 | atgcagaata aaacaaatta taaacatgtg gcagaaagta |
| | ggaaaagcaa tgtatgcccc |
| 7081 | tcccatcagt ggacaaatta gatgttcatc aaatattaca |
| | gggctgctat taacaagaga |
| 7141 | tggtggtaat agcaacaatg agtccgagat cttcagacct |
| | ggaggaggag atatgaggga |
| 7201 | caattggaga agtgaattat ataaatataa agtagtaaaa |
| | attgaaccat taggagtagc |
| 7261 | acccaccaag gcaaagagaa gagtggtgca gagagaaaaa |
| | agagcagtgg gaataggagc |
| 7321 | tttgttcctt gggttcttgg gagcagcagg aagcactatg |
| | ggcgcagcct caatgacgct |
| 7381 | gacggtacag gccagacaat tattgtctgg tatagtgcag |
| | cagcagaaca atttgctgag |
| 7441 | ggctattgag gcgcaacagc atctgttgca actcacagtc |
| | tggggcatca agcagctcca |
| 7501 | ggcaagaatc ctggctgtgg aaagatacct aaaggatcaa |
| | cagctcctgg ggatttgggg |
| 7561 | ttgctctgga aaactcattt gcaccactgc tgtgccttgg |
| | aatgctagtt ggagtaataa |
| 7621 | atctctggaa cagatttgga atcacacgac ctggatggag |
| | tgggacagag aaattaacaa |
| 7681 | ttacacaagc ttaatacact ccttaattga agaatcgcaa |
| | aaccagcaag aaaagaatga |
| 7741 | acaagaatta ttggaattag ataaatgggc aagtttgtgg |
| | aattggttta acataacaaa |
| 7801 | ttggctgtgg tatataaaat tattcataat gatagtagga |
| | ggcttggtag gtttaagaat |
| 7861 | agtttttgct gtactttcta tagtgaatag agttaggcag |
| | ggatattcac cattatcgtt |
| 7921 | tcagacccac ctcccaaccc cgaggggacc cgacaggccc |
| | gaaggaatag aagaagaagg |
| 7981 | tggagagaga gacagagaca gatccattcg attagtgaac |
| | ggatccttgg cacttatctg |
| 8041 | ggacgatctg cggagcctgt gcctcttcag ctaccaccgc |
| | ttgagagact tactcttgat |
| 8101 | tgtaacgagg attgtggaac ttctgggacg cagggggtgg |
| | gaagccctca aatattggtg |
| 8161 | gaatctccta cagtattgga gtcaggaact aaagaatagt |
| | gctgttagct tgctcaatgc |
| 8221 | cacagccata gcagtagctg aggggacaga tagggttata |
| | gaagtagtac aaggagcttg |
| 8281 | tagagctatt cgccacatac ctagaagaat aagacagggc |
| | ttggaaagga ttttgctata |
| 8341 | agatgggtgg caagtggtca aaaagtagtg tgattggatg |
| | gcctactgta agggaaagaa |
| 8401 | tgagacgagc tgagccagca gcagataggg tgggagcagc |
| | atctcgagac ctggaaaaac |
| 8461 | atggagcaat cacaagtagc aatacagcag ctaccaatgc |
| | tgcttgtgcc tggctagaag |
| 8521 | cacaagagga ggaggaggtg ggttttccag tcacacctca |
| | ggtaccttta agaccaatga |
| 8581 | cttacaaggc agctgtagat cttagccact ttttaaaaga |
| | aaagggggga ctggaagggc |
| 8641 | taattcactc ccaaagaaga caagatatcc ttgatctgtg |
| | gatctaccac acacaaggct |
| 8701 | acttccctga ttagcagaac tacacaccag ggccaggggt |
| | cagatatcca ctgacctttg |
| 8761 | gatggtgcta caagctagta ccagttgagc cagataagat |
| | agaagaggcc aataaaggag |
| 8821 | agaacaccag cttgttacac cctgtgagcc tgcatgggat |
| | ggatgacccg gagagagaag |
| 8881 | tgttagagtg gaggtttgac agccgcctag catttcatca |
| | cgtggcccga gagctgcatc |
| 8941 | cggagtactt caagaactgc tgacatcgag cttgctacaa |
| | gggactttcc gctggggact |
| 9001 | ttccagggag gcgtggcctg ggcgggactg gggagtggcg |
| | agccctcaga tcctgcatat |
| 9061 | aagcagctgc tttttgcctg tactgggtct ctctggttag |
| | accagatctg agcctgggag |
| 9121 | ctctctggct aactagggaa cccactgctt aagcctcaat |
| | aaagcttgcc ttgagtgctt |
| 9181 | c |

Initial Specific Target Motifs:
(1) Trans-activation response region/Tat protein binding site—TAR RNA—nts 1-60

“Minimal” TAR RNA Element

| | |
|---|---|
| 5′ | |
| GGCAGAUCUGAGCCUGGGAGCUCUCUGCC 3′ | (SEQ ID NO: 15) |

(2) Gag/Pol Frameshifting Site—“Minimal” frameshifting element

(SEQ ID NO: 16)

5′ UUUUUUAGGGAAGAUCUGGCCUUCCUACAAGGGAAGGCCAGG
GAAUUUUCUU 3′

### 5.7. Hepatitis C Virus (“HCV”—Genotypes 1a & 1b)

GenBank Accession # NC_{—}001433:

(SEQ ID NO: 17)

| | |
|---|---|
| 1 | ttgggggcga cactccacca tagatcactc ccctgtgagg |
| | aactactgtc ttcacgcaga |
| 61 | aagcgtctag ccatggcgtt agtatgagtg ttgtgcagcc |
| | tccaggaccc cccctcccgg |
| 121 | gagagccata gtggtctgcg gaaccggtga gtacaccgga |
| | attgccagga cgaccgggtc |
| 181 | ctttcttgga tcaacccgct caatgcctgg agatttgggc |
| | gtgcccccgc gagactgcta |
| 241 | gccgagtagt gttgggtcgc gaaaggcctt gtggtactgc |
| | ctgatagggt gcttgcgagt |
| 301 | gccccgggag gtctcgtaga ccgtgcatca tgagcacaaa |
| | tcctaaacct caaagaaaaa |
| 361 | ccaaacgtaa caccaaccgc cgcccacagg acgttaagtt |
| | cccgggcggt ggtcagatcg |
| 421 | ttggtggagt ttacctgttg ccgcgcaggg gccccaggtt |
| | gggtgtgcgc gcgactagga |
| 481 | agacttccga gcggtcgcaa cctcgtggaa ggcgacaacc |
| | tatccccaag gctcgccggc |
| 541 | ccgagggtag gacctgggct cagcccgggt acccttggcc |
| | cctctatggc aacgagggta |
| 601 | tggggtgggc aggatggctc ctgtcacccc gtggctctcg |
| | gcctagttgg ggccccacag |
| 661 | acccccggcg taggtcgcgt aatttgggta aggtcatcga |
| | tacccttaca tgcggcttcg |
| 721 | ccgacctcat ggggtacatt ccgcttgtcg gcgcccccct |
| | agggggcgct gccagggccc |
| 781 | tggcacatgg tgtccgggtt ctggaggacg gcgtgaacta |
| | tgcaacaggg aatctgcccg |
| 841 | gttgctcttt ctctatcttc ctcttagctt tgctgtcttg |
| | tttgaccatc ccagcttccg |
| 901 | cttacgaggt gcgcaacgtg accgggatat accatgtcac |
| | gaacgactgc tccaactcaa |
| 961 | gtattgtgta tgaggcagcg tccatgatca tgcacacccc |
| | cgggtgcgtg ccctgcgtcc |
| 1021 | gggagagtaa tttctcccgt tgctgggtag cgctcactcc |
| | cacgctcgcg gccaggaaca |
| 1081 | gcagcatccc caccacgaca atacgacgcc acgtcgattt |
| | gctcgttggg gcggctgctc |
| 1141 | tctgttccgc tatgtacgtt ggggatctct gcggatccgt |
| | ttttctcgtc tcccagctgt |
| 1201 | tcaccttctc acctcgccgg tatgagacgg tacaagattg |
| | caattgctca atctatcccg |
| 1261 | gccacgtatc aggtcaccgc atggcttggg atatgatgat |
| | gaactggtca cctacaacgg |
| 1321 | ccctagtggt atcgcagcta ctccggatcc cacaagccgt |
| | cgtggacatg gtggcggggg |
| 1381 | cccactgggg tgtcctagcg ggccttgcct actattccat |
| | ggtggggaac tgggctaagg |
| 1441 | tcttgattgt gatgctactc tttgctggcg ttgacgggca |
| | cacccacgtg acagggggaa |
| 1501 | gggtagcctc cagcacccag agcctcgtgt cctggctctc |
| | acaaggccca tctcagaaaa |
| 1561 | tccaactcgt gaacaccaac ggcagctggc acatcaacag |
| | gaccgctctg aattgcaatg |
| 1621 | actccctcca aactgggttc attgctgcgc tgttctacgc |
| | acacaggttc aacgcgtccg |
| 1681 | ggtgcccaga gcgcatggct agctgccgcc ccatcgatga |
| | gttcgctcag gggtggggtc |
| 1741 | ccatcactca tgatatgcct gagagctcgg accagaggcc |
| | atattgctgg cactacgcgc |
| 1801 | ctcgaccgtg cgggatcgtg cctgcgtcgc aggtgtgtgg |
| | tccagtgtat tgcttcactc |
| 1861 | cgagccctgt tgtagtgggg acgaccgatc gtttcggcgc |
| | tcctacgtat agctgggggg |
| 1921 | agaatgagac agacgtgctg ctacttagca acacgcggcc |
| | gcctcaaggc aactggtttg |
| 1981 | ggtgcacgtg gatgaacagc actgggttca ccaagacgtg |
| | cgggggccct ccgtgcaaca |
| 2041 | tcgggggggt cggcaacaac accttggtct gccccacgga |
| | ttgcttccgg aagcaccccg |
| 2101 | aggccactta cacaaagtgt ggctcggggc cctggttgac |
| | acccaggtgc atggttgact |
| 2161 | acccatacag gctctggcac tacccctgca ctgttaactt |
| | taccgtcttt aaggtcagga |
| 2221 | tgtatgtggg gggcgtggag cacaggctca atgctgcatg |
| | caattggact cgaggagagc |
| 2281 | gctgtgactt ggaggacagg gataggtcag aactcagccc |
| | gctgctgctg tctacaacag |
| 2341 | agtggcagat actgccctgt tccttcacca ccctaccggc |
| | cctgtccact ggcttgatcc |
| 2401 | atcttcaccg gaacatcgtg gacgtgcaat acctgtacgg |
| | tatagggtcg gcagttgtct |
| 2461 | cctttgcaat caaatgggag tatatcctgt tgcttttcct |
| | tcttctggcg gacgcgcgcg |
| 2521 | tctgtgcctg cttgtggatg atgctgctga tagcccaggc |
| | tgaggccacc ttagagaacc |
| 2581 | tggtggtcct caatgcggcg tctgtggccg gagcgcatgg |
| | ccttctctcc ttcctcgtgt |
| 2641 | tcttctgcgc cgcctggtac atcaaaggca ggctggtccc |
| | tggggcggca tatgctctct |
| 2701 | atggcgtatg gccgttgctc ctgctcttgc tggccttacc |
| | accacgagct tatgccatgg |
| 2761 | accgagagat ggctgcatcg tgcggaggcg cggtttttgt |
| | aggtctggta ctcttgacct |
| 2821 | tgtcaccata ctataaggtg ttcctcgcta ggctcatatg |
| | gtggttacaa tattttatca |
| 2881 | ccagagccga ggcgcacttg caagtgtggg tcccccctct |
| | caatgttcgg ggaggccgcg |
| 2941 | atgccatcat cctccttaca tgcgcggtcc atccagagct |
| | aatctttgac atcaccaaac |
| 3001 | tcctgctcgc catactcggt ccgctcatgg tgctccaggc |
| | tggcataact agagtgccgt |
| 3061 | actttgtacg cgctcagggg ctcatccgtg catgcatgtt |
| | agtgcggaag gtcgctggag |
| 3121 | gccactatgt ccaaatggcc ttcatgaagc tggccgcgct |
| | gacaggtacg tacgtatatg |
| 3181 | accatcttac tccactgcgg gattgggccc acgcgggcct |
| | acgagacctt gcggtggcag |
| 3241 | tagagcccgt cgtcttctct gacatggaga ctaaactcat |
| | cacctggggg gcagacaccg |
| 3301 | cggcgtgtgg ggacatcatc tcgggtctac cagtctccgc |
| | ccgaaggggg aaggagatac |
| 3361 | ttctaggacc ggccgatagt tttggagagc aggggtggcg |
| | gctccttgcg cctatcacgg |
| 3421 | cctattccca acaaacgcgg ggcctgcttg gctgtatcat |
| | cactagcctc acaggtcggg |
| 3481 | acaagaacca ggtcgatggg gaggttcagg tgctctccac |
| | cgcaacgcaa tctttcctgg |
| 3541 | cgacctgcgt caatggcgtg tgttggaccg tctaccatgg |
| | tgccggctcg aagaccctgg |
| 3601 | ccggcccgaa gggtccaatc acccaaatgt acaccaatgt |
| | agaccaggac ctcgtcggct |
| 3661 | ggccggcgcc ccccggggcg cgctccatga caccgtgcac |
| | ctgcggcagc tcggaccttt |
| 3721 | acttggtcac gaggcatgct gatgtcgttc cggtgcgccg |
| | gcggggcgac agcaggggga |
| 3781 | gcctgctttc ccccaggccc atctcctacc tgaagggctc |
| | ctcgggtgga ccactgcttt |
| 3841 | gcccttcggg gcacgttgta ggcatcttcc gggctgctgt |
| | gtgcacccgg ggggttgcga |
| 3901 | aggcggtgga cttcataccc gttgagtcta tggaaactac |
| | catgcggtct ccggtcttca |
| 3961 | cagacaactc atcccctccg gccgtaccgc aaacattcca |
| | agtggcacat ttacacgctc |
| 4021 | ccactggcag cggcaagagc accaaagtgc cggctgcata |
| | tgcagcccaa gggtacaagg |
| 4081 | tgctcgtcct aaacccgtcc gttgccgcca cattgggctt |
| | tggagcgtat atgtccaagg |
| 4141 | cacatggcat cgagcctaac atcagaactg gggtaaggac |
| | catcaccacg ggcggcccca |
| 4201 | tcacgtactc cacctattgc aagttccttg ccgacggtgg |
| | atgctccggg ggcgcctatg |
| 4261 | acatcataat atgtgatgaa tgccactcaa ctgactcgac |
| | taccatcttg ggcatcggca |
| 4321 | cagtcctgga tcaggcagag acggctggag cgcggctcgt |
| | cgtgctcgcc accgccacgc |
| 4381 | ctccgggatc gatcaccgtg ccacacccca acatcgagga |
| | agtggccctg tccaacactg |
| 4441 | gagagattcc cttctatggc aaagccatcc ccattgaggc |
| | catcaagggg ggaaggcatc |
| 4501 | tcatcttctg ccattccaag aagaagtgtg acgagctcgc |
| | cgcaaagctg acaggcctcg |
| 4561 | gactcaatgc tgtagcgtat taccggggtc tcgatgtgtc |
| | cgtcataccg actagcggag |
| 4621 | acgtcgttgt cgtggcaaca gacgctctaa tgacgggttt |
| | taccggcgac tttgactcag |
| 4681 | tgatcgactg caacacatgt gtcacccaga cagtcgattt |
| | cagcttggat cccaccttca |
| 4741 | ccattgagac gacaacgctg ccccaagacg cggtgtcgcg |
| | tgcgcagcgg cgaggtagga |
| 4801 | ctggcagggg caggagtggc atctacaggt ttgtgactcc |
| | aggagaacgg ccctcaggca |
| 4861 | tgttcgactc ctcggtcctg tgtgagtgct atgacgcagg |
| | ctgcgcttgg tatgagctca |
| 4921 | cgcccgctga gacctcggtt aggttgcggg cttacctaaa |
| | tacaccaggg ttgcccgtct |
| 4981 | gccaggacca cctagagttc tgggagagcg tcttcacagg |
| | cctcacccac atagatgccc |
| 5041 | acttcttgtc ccagaccaaa caggcaggag acaacctccc |
| | ctacctggta gcataccaag |
| 5101 | ccacagtgtg cgccagggct caggctccac ctccatcgtg |
| | ggaccaaatg tggaagtgtc |
| 5161 | tcatacggct aaagcccaca ctgcatgggc caacgcccct |
| | gctgtacagg ctaggagccg |
| 5221 | ttcaaaatga ggtcactctc acacacccca taaccaaata |
| | catcatggca tgcatgtcgg |
| 5281 | ctgacctgga ggtcgtcact agcacctggg tgctagtagg |
| | cggagtcctt gcggctctgg |
| 5341 | ccgcgtactg cctgacgaca ggcagcgtgg tcattgtggg |
| | caggatcatc ttgtccggga |
| 5401 | ggccagctgt tattcccgac agggaagtcc tctaccagga |
| | gttcgatgag atggaagagt |
| 5461 | gtgcttcaca cctcccttac atcgagcaag gaatgcagct |
| | cgccgagcaa ttcaaacaga |
| 5521 | aggcgctcgg attgctgcaa acagccacca agcaagcgga |
| | ggctgctgct cccgtggtgg |
| 5581 | agtccaagtg gcgagccctt gaggtcttct gggcgaaaca |
| | catgtggaac ttcatcagcg |
| 5641 | ggatacagta cttggcaggc ctatccactc tgcctggaaa |
| | ccccgcgata gcatcattga |
| 5701 | tggcttttac agcctctatc accagcccgc tcaccaccca |
| | aaataccctc ctgtttaaca |
| 5761 | tcttgggggg atgggtggct gcccaactcg ctccccccag |
| | cgctgcttcg gctttcgtgg |
| 5821 | gcgccggcat tgccggtgcg gccgttggca gcataggtct |
| | cgggaaggta cttgtggaca |
| 5881 | ttctggcggg ctatggggcg ggggtggctg gcgcactcgt |
| | ggcctttaag gtcatgagcg |
| 5941 | gcgagatgcc ctccactgag gatctggtta atttactccc |
| | tgccatcctt tctcctggcg |
| 6001 | ccctggttgt cggggtcgtg tgcgcagcaa tactgcgtcg |
| | gcacgtgggc ccgggagagg |
| 6061 | gggctgtgca gtggatgaac cggctgatag cgttcgcttc |
| | gcggggtaac cacgtctccc |
| 6121 | ccacgcacta tgtgcccgag agcgacgccg cggcgcgtgt |
| | tactcagatc ctctccagcc |
| 6181 | ttaccatcac tcagttgctg aagaggcttc atcagtggat |
| | taatgaggac tgctccacgc |
| 6241 | cttgttccgg ctcgtggcta aaggatgttt gggactggat |
| | atgcacggtg ttgagtgact |
| 6301 | tcaagacttg gctccagtcc aagctcctgc cgcggttacc |
| | gggactccct ttcctgtcat |
| 6361 | gccaacgcgg gtacaaggga gtctggcggg gggatggcat |
| | catgcaaacc acctgcccat |
| 6421 | gtggagcaca gatcaccgga catgtcaaaa atggctccat |
| | gaggattgtt gggccaaaaa |
| 6481 | cctgcagcaa cacgtggcat ggaacattcc ccatcaacgc |
| | atacaccacg ggcccctgca |
| 6541 | cgccctcccc agcgccgaac tattccaggg cgctgtggcg |
| | ggtggctgct gaggagtacg |
| 6601 | tggaggttac gcgggtgggg gatttccact acgtgacggg |
| | catgaccact gacaacgtga |
| 6661 | aatgcccatg ccaggttcca gcccctgaat ttttcacgga |
| | ggtggatgga gtacggttgc |
| 6721 | acaggtatgc tccagtgtgc aaacctctcc tacgagagga |
| | ggtcgtattc caggtcgggc |
| 6781 | tcaaccagta cctggtcggg tcacagctcc catgtgagcc |
| | cgaaccggat gtggcagtgc |
| 6841 | tcacttccat gctcaccgac ccctctcata ttacagcaga |
| | gacggccaag cgtaggctgg |
| 6901 | ccagggggtc tcccccctcc ttggccagct cttcagctag |
| | ccagttgtct gcgccttctt |
| 6961 | tgaaggcgac atgtactacc catcatgact ccccggacgc |
| | tgacctcatc gaggccaacc |
| 7021 | tcctgtggcg gcaggagatg ggcgggaaca tcacccgtgt |
| | ggagtcagaa aataaggtgg |
| 7081 | taatcctgga ctctttcgat ccgattcggg cggtggagga |
| | tgagagggaa atatccgtcc |
| 7141 | cggcggagat cctgcgaaaa cccaggaagt tccccccagc |
| | gttgcccata tgggcacgcc |
| 7201 | cggattacaa ccctccactg ctagagtcct ggaaggaccc |
| | ggactacgtc cccccggtgg |
| 7261 | tacacgggtg ccctttgcca tctaccaagg cccccccaat |
| | accacctcca cggaggaaga |
| 7321 | ggacggttgt cctgacagag tccaccgtgt cttctgcctt |
| | ggcggagctc gctactaaga |
| 7381 | cctttggcag ctccgggtcg tcggccgttg acagcggcac |
| | ggcgactggc cctcccgatc |
| 7441 | aggcctccga cgacggcgac aaaggatccg acgttgagtc |
| | gtactcctcc atgccccccc |
| 7501 | tcgagggaga gccaggggac cccgacctca gcgacgggtc |
| | ttggtctacc gtgagcgggg |
| 7561 | aagctggtga ggacgtcgtc tgctgctcaa tgtcctatac |
| | atggacaggt gccttgatca |
| 7621 | cgccatgcgc tgcggaggag agcaagttgc ccatcaatcc |
| | gttgagcaac tctttgctgc |
| 7681 | gtcaccacag tatggtctac tccacaacat ctcgcagcgc |
| | aagtctgcgg cagaagaagg |
| 7741 | tcacctttga cagactgcaa gtcctggacg accactaccg |
| | ggacgtgctc aaggagatga |
| 7801 | aggcgaaggc gtccacagtt aaggctaggc ttctatctat |
| | agaggaggcc tgcaaactga |
| 7861 | cgcccccaca ttcggccaaa tccaaatttg gctacggggc |
| | gaaggacgtc cggagcctat |
| 7921 | ccagcagggc cgtcaaccac atccgctccg tgtgggagga |
| | cttgctggaa gacactgaaa |
| 7981 | caccaattga taccaccatc atggcaaaaa atgaggtttt |
| | ctgcgtccaa ccagagaaag |
| 8041 | gaggccgcaa gccagctcgc cttatcgtat tcccagacct |
| | gggggtacgt gtatgcgaga |
| 8101 | agatggccct ttacgacgtg gtctccaccc ttcctcaggc |
| | cgtgatgggc ccctcatacg |
| 8161 | gattccagta ctctcctggg cagcgggtcg agttcctggt |
| | gaatacctgg aaatcaaaga |
| 8221 | aatgccctat gggcttctca tatgacaccc gctgctttga |
| | ctcaacggtc actgagaatg |
| 8281 | acatccgtac tgaggaatca atttaccaat gttgtgactt |
| | ggcccccgaa gccaggcagg |
| 8341 | ccataaggtc gctcacagag cggctttatg tcgggggtcc |
| | cctgactaat tcgaaggggc |
| 8401 | agaactgcgg ttatcgccgg tgccgcgcaa gtggcgtgct |
| | gacgactagc tgcggcaaca |
| 8461 | ccctcacatg ttacttgaag gccactgcgg cctgtcgagc |
| | tgcaaagctc caggactgca |
| 8521 | cgatgctcgt gaacggagac gaccttgtcg ttatctgtga |
| | gagtgcggga acccaggagg |
| 8581 | atgcggcggc cctacgagcc ttcacggagg ctatgactag |
| | gtattccgcc ccccccgggg |
| 8641 | acccgcccca accagaatac gacttggagc tgataacgtc |
| | atgctcctcc aatgtgtcgg |
| 8701 | tcgcgcacga tgcatccggc aaaagggtgt actacctcac |
| | ccgtgacccc accacccccc |
| 8761 | tcgcacgggc tgcgtgggag acagttagac acactccagt |
| | caactcctgg ctaggcaata |
| 8821 | tcatcatgta tgcgcccacc ctatgggcga ggatgattct |
| | gatgactcat ttcttctcta |
| 8881 | tccttctagc tcaggagcaa cttgaaaaag ccctggattg |
| | tcagatctac ggggcctgtt |
| 8941 | actccattga gccacttgac ctacctcaga tcattgaacg |
| | actccatggt cttagcgcat |
| 9001 | tttcactcca cagttactct ccaggtgaga tcaatagggt |
| | ggcttcatgc ctcaggaaac |
| 9061 | ttggggtacc gcctttgcga gtctggagac atcgggccag |
| | aagtgtccgc gctaagctac |
| 9121 | tgtcccaggg ggggagggct gccacttgcg gcaagtacct |
| | cttcaactgg gcagtaaaga |
| 9181 | ccaagcttaa actcactcca atcccggctg cgtcccagct |
| | agacttgtcc ggctggttcg |
| 9241 | ttgctggtta caacggggga gacatatatc acagcctgtc |
| | tcgtgcccga ccccgttggt |
| 9301 | tcatgttgtg cctactccta ctttctgtag gggtaggcat |
| | ctacctgctc cccaaccggt |
| 9361 | gaacggggag ctaaccactc caggccaata ggccattccc |
| | tttttttttt ttc |

General Target Region:

5′ Untranslated Region—nts 1-328—Internal Ribosome Entry Site (IRES):

| | |
|---|---|
| 5′UUGGGGGCGACACUCCACCAUAGAUCACUCCCCUGUGAGGAACUACUGUCUU | (SEQ ID NO: 18) |
| CACGCAGAAAGCGUCUAGCCAUGGCGUUAGUAUGAGUGUUGUGCAGCCUCCA | |
| GGACCCCCCCUCCCGGGAGAGCCAUAGUGGUCUGCGGAACCGGUGAGUACACC | |
| GGAAUUGCCAGGACGACCGGGUCCUUUCUUGGAUCAACCCGCUCAAUGCCUGG | |
| AGAUUUGGGCGUGCCCCCGCGAGACUGCUAGCCGAGUAGUGUUGGGUCGCGA | |
| AAGGCCUUGUGGUACUGCCUGAUAGGGUGCUUGCGAGUGCCCCGGGAGGUCU | |
| CGUAGACCGUGCAU3′ | |

Initial Specific Target Motifs:

(1) Subdomain IIIc within HCV IRES—nts 213-226

| | |
|---|---|
| 5′AUUUGGGCGUGCCC3′ | (SEQ ID NO: 19) |

(2) Subdomain IIId within HCV IRES—nts 241-267

| | |
|---|---|
| 5′GCCGAGUAGUGUUGGGUCGCGAAAGGC3′ | (SEQ ID NO: 20) |

### 5.8. Ribonuclease P RNA (“RNaseP”)

GenBank Accession #s

X15624 Homo sapiens RNaseP H1 RNA:

(SEQ ID NO: 21)

| | |
|---|---|
| 1 | atgggcggag ggaagctcat cagtggggcc acgagctgag |
| | tgcgtcctgt cactccactc |
| 61 | ccatgtccct tgggaaggtc tgagactagg gccagaggcg |
| | gccctaacag ggctctccct |
| 121 | gagcttcagg gaggtgagtt cccagagaac ggggctccgc |
| | gcgaggtcag actgggcagg |
| 181 | agatgccgtg gaccccgccc ttcggggagg ggcccggcgg |
| | atgcctcctt tgccggagct |
| 241 | tggaacagac tcacggccag cgaagtgagt tcaatggctg |
| | aggtgaggta ccccgcaggg |
| 301 | gacctcataa cccaattcag accactctcc tccgcccatt |

U64885 Staphylococcus aureus RNaseP (rrnB) RNA:

(SEQ ID NO: 22)

| | |
|---|---|
| 1 | gaggaaagtc cgggctcaca cagtctgaga tgattgtagt |
| | gttcgtgctt gatgaaacaa |
| 61 | taaatcaagg cattaatttg acggcaatga aatatcctaa |
| | gtctttcgat atggatagag |
| 121 | taatttgaaa gtgccacagt gacgtagctt ttatagaaat |
| | ataaaaggtg gaacgcggta |
| 181 | aacccctcga gtgagcaatc caaatttggt aggagcactt |
| | gtttaacgga attcaacgta |
| 241 | taaacgagac acacttcgcg aaatgaagtg gtgtagacag |
| | atggttatca cctgagtacc |
| 301 | agtgtgacta gtgcacgtga tgagtacgat ggaacagaac |
| | gcggcttat |

M17569 Escherichia coli RNA component (M1 RNA) of ribonuclease P (rnpB) gene:

(SEQ ID NO: 23)

| | |
|---|---|
| 1 | gaagctgacc agacagtcgc cgcttcgtcg tcgtcctctt |
| | cgggggagac gggcggaggg |
| 61 | gaggaaagtc cgggctccat agggcagggt gccaggtaac |
| | gcctgggggg gaaacccacg |
| 121 | accagtgcaa cagagagcaa accgccgatg gcccgcgcaa |
| | gcgggatcag gtaagggtga |
| 181 | aagggtgcgg taagagcgca ccgcgcggct ggtaacagtc |
| | cgtggcacgg taaactccac |
| 241 | ccggagcaag gccaaatagg ggttcataag gtacggcccg |
| | tactgaaccc gggtaggctg |
| 301 | cttgagccag tgagcgattg ctggcctaga tgaatgactg |
| | tccacgacag aacccggctt |
| 361 | atcggtcagt ttcacct |

Z70692 Mycobacterium tuberculosis RNaseP (rnpB) RNA:

(SEQ ID NO: 24)

| | |
|---|---|
| 1 | ccaccggtta cgatcttgcc gaccatggcc ccacaatagg |
| | gccggggaga cccggcgtca |
| 61 | gtggtgggcg gcacggtcag taacgtctgc gcaacacggg |
| | gttgactgac gggcaatatc |
| 121 | ggctccatag cgtcggccgc ggatacagta aaggagcatt |
| | ctgtgacgga aaagacgccc |
| 181 | gacgacgtct tcaaacttgc caaggacgag aaggtcgaat |
| | atgtcgacgt ccggttctgt |
| 241 | gacctgcctg gcatcatgca gcacttcacg attccggctt |
| | cggcctttga caagagcgtg |
| 301 | tttgacgacg gcttggcctt tgacggctcg tcgattcgcg |
| | ggttccagtc gatccacgaa |
| 361 | tccgacatgt tgcttcttcc cgatcccgag acggcgcgca |
| | tcgacccgtt ccgcgcggcc |
| 421 | aagacgctga atatcaactt ctttgtgcac gacccgttca |
| | ccctggagcc gtactcccgc |
| 481 | gacccgcgca acatcgcccg caaggccgag aactacctga |
| | tcagcactgg catcgccgac |
| 541 | accgcatact tcggcgccga ggccgagttc tacattttcg |
| | attcggtgag cttcgactcg |
| 601 | cgcgccaacg gctccttcta cgaggtggac gccatctcgg |
| | ggtggtggaa caccggcgcg |
| 661 | gcgaccgagg ccgacggcag tcccaaccgg ggctacaagg |
| | tccgccacaa gggcgggtat |
| 721 | ttcccagtgg cccccaacga ccaatacgtc gacctgcgcg |
| | acaagatgct gaccaacctg |
| 781 | atcaactccg gcttcatcct ggagaagggc caccacgagg |
| | tgggcagcgg cggacaggcc |
| 841 | gagatcaact accagttcaa ttcgctgctg cacgccgccg |
| | acgacatgca gttgtacaag |
| 901 | tacatcatca agaacaccgc ctggcagaac ggcaaaacgg |
| | tcacgttcat gcccaagccg |
| 961 | ctgttcggcg acaacgggtc cggcatgcac tgtcatcagt |
| | cgctgtggaa ggacggggcc |
| 1021 | ccgctgatgt acgacgagac gggttatgcc ggtctgtcgg |
| | acacggcccg tcattacatc |
| 1081 | ggcggcctgt tacaccacgc gccgtcgctg ctggccttca |
| | ccaacccgac ggtgaactcc |
| 1141 | tacaagcggc tggttcccgg ttacgaggcc ccgatcaacc |
| | tggtctatag ccagcgcaac |
| 1201 | cggtcggcat gcgtgcgcat cccgatcacc ggcagcaacc |
| | cgaaggccaa gcggctggag |
| 1261 | ttccgaagcc ccgactcgtc gggcaacccg tatctggcgt |
| | tctcggccat gctgatggca |
| 1321 | ggcctggacg gtatcaagaa caagatcgag ccgcaggcgc |
| | ccgtcgacaa ggatctctac |
| 1381 | gagctgccgc cggaagaggc cgcgagtatc ccgcagactc |
| | cgacccagct gtcagatgtg |
| 1441 | atcgaccgtc tcgaggccga ccacgaatac ctcaccgaag |
| | gaggggtgtt cacaaacgac |
| 1501 | ctgatcgaga cgtggatcag tttcaagcgc gaaaacgaga |
| | tcgagccggt caacatccgg |
| 1561 | ccgcatccct acgaattcgc gctgtactac gacgtttaag |
| | gactcttcgc agtccgggtg |
| 1621 | tagagggagc ggcgtgtcgt tgccagggcg ggcgtcgagg |
| | tttttcgatg ggtgacggtg |
| 1681 | gccggcaacg gcgcgccgac caccgctgcg aagagcccgt |
| | ttaagaacgt tcaaggacgt |
| 1741 | ttcagccggg tgccacaacc cgcttggcaa tcatctcccg |
| | accgccgagc gggttgtctt |
| 1801 | tcacatgcgc cgaaactcaa gccacgtcgt cgcccaggcg |
| | tgtcgtcgcg gccggttcag |
| 1861 | gttaagtgtc ggggattcgt cgtgcgggcg ggcgtccacg |
| | ctgaccaacg gggcagtcaa |
| 1921 | ctcccgaaca ctttgcgcac taccgccttt gcccgccgcg |
| | tcacccgtag gtagttgtcc |
| 1981 | aggaattccc caccgtcgtc gtttcgccag ccggccgcga |
| | ccgcgaccgc attgagctgg |
| 2041 | cgcccgggtc ccggcagctg gtcggtgggc ttgccgcgca |
| | ccaacaccag cgcgttgcgg |
| 2101 | gcccgggtgg cggtcagcca ggcctgacgg agcagctcca |
| | cgtcggctgc gggaaccaga |
| 2161 | tcggcggccg cgatgacatc cagggattgc agcgtcgagg |
| | tgttgtgcag ggcgggaacc |
| 2221 | tggtgcgcat gctgtagctg cagcaactgc acggtccatt |
| | cgatgtcggc cagtccgccg |
| 2281 | cggcccagtt tggtgtgtgt gttggggtcg gcaccgcgcg |
| | gcaaccgctc ggactcgata |
| 2341 | cgggccttga tgcggcgaat ctcgcgcacc gagtcagcgg |
| | acacaccgtc gggcggatac |
| 2401 | cgcgttttgt cgaccatccg taggaatcgc tgacccaact |
| | cggcatcgcc ggcaaccgcg |
| 2461 | tgtgcgcgta gcagggcctg gatctcccat ggctgtgccc |
| | actgctcgta gtatgcggcg |
| 2521 | taggacccca gggtgcggac cagcggaccg ttgcggccct |
| | cgggtcgcaa attggcgtcg |
| 2581 | agctccagcg gcggatcgac gctgggtgtc cccagcagcg |
| | cccgaacccg ctcggcgatc |
| 2641 | gatgtcgacc atttcaccgc ccgtgcatcg tcgacgccgg |
| | tggccggctc acagacgaac |
| 2701 | atcacgtcgg catccgaccc gtagcccaac tcggcaccac |
| | ccagccgacc catgccgatg |
| 2761 | accgcgatgg ccgccggggc gcgatcgtcg tcgggaaggc |
| | tggcccggat catgacgtcc |
| 2821 | agcgcggcct gcagcaccgc cacccacacc gacgtcaacg |
| | cccggcacac ctcggtgacc |
| 2881 | tcgagcaggc cgagcaggtc cgccgaaccg atgcgggcca |
| | gctctcgacg acgcagcgtg |
| 2941 | cgcgcgccgg cgatggcccg ctccgggtcg gggtagcggc |
| | tcgccgaggc gatcagcgcc |
| 3001 | cgagccacgg cggcgggctc ggtctcgagc agcttcgggc |
| | ccgcaggccc gtcctcgtac |
| 3061 | tgctggatga cccgcggcgc gcgcatcaac agatccggca |
| | catacgccga ggtacccaag |
| 3121 | acatgcatga gccgcttggc caccgcgggc ttgtcccgca |
| | gcgtggccag gtaccagctt |
| 3181 | tcggtggcca gcgcctcact gagccgccgg taggccagca |
| | gtccgccgtc gggatcgggg |
| 3241 | gcatacgaca tccagtccag cagcctgggc agcagcaccg |
| | actgcacccg tccgcgccgg |
| 3301 | ccgctttgat tgaccaacgc cgacatgtgt ttcaacgcgg |
| | tctgcggtcc ctcgtagccc |
| 3361 | agcgcggcca gccggcgccc cgcggcctcc aacgtcatgc |
| | cgtgggcgat ctccaacccg |
| 3421 | gtcgggccga tcgattccag cagcggttga tagaagagtt |
| | tggtgtgtaa cttcgacacc |
| 3481 | cgcacgttct gcttcttgag ttcctcccgc agcaccccgg |
| | ccgcatcgtt tcggccatcg |
| 3541 | ggccggatgt gggccgcgcg cgccagccag cgcactgcct |
| | cctcgtcttc gggatcggga |
| 3601 | agcaggtggg tgcgcttgag ccgctgcaac tgcagtcggt |
| | gctcgagcag cctgaggaac |
| 3661 | tcatacgacg cggtcatgtt cgccgcgtcc tcacgcccga |
| | tgtagccgcc ttcgcccaac |
| 3721 | gccgccaatg cgtccaccgt ggacgccacc cgtaacgact |
| | cgtcgctacg ggcatgaacc |
| 3781 | agctgcagta gctgtacggc gaactccacg tcgcgcaatc |
| | cgccgctgcc gagtttgagc |
| 3841 | tcgcggccgc ggacatcggc gggcaccagc tgctccaccc |
| | gccgccgcat ggcctgcacc |
| 3901 | tcgaccacaa agtcttcgcg ctcgcaggct cgccacacca |
| | tcggcatcaa ggcggtcagg |
| 3961 | taacgctcgc caagttccgc gtcgccaacg actggccgtg |
| | ctttcagcaa cgcctgaaac |
| 4021 | tcccaggtct tggcccagcg ctggtagtag gcgatgtgcg |
| | actcgagcgt acggaccagc |
| 4081 | tccccgttgc gcccctccgg acgcagggcg gcgtccacct |
| | cgaaaaaggc cgccgaggcc |
| 4141 | acccgcatca tctcgctggc cacgcgcgcg ttgcgcgggt |
| | cggagcgctc ggcaacgaat |
| 4201 | atgacatcga cgtcgctgac gtagttcagt tcgcgcgcac |
| | cgcacttgcc catcgcgatg |
| 4261 | accgccaggc gcggtggcgg gtgctcgccg cacacgctcg |
| | cctcggccac gcgcagcgcc |
| 4321 | gccgccagag cggcgtccgc ggcgtccgcc aggcgtgcgg |
| | ccaccacggt gaatggcagc |
| 4381 | accggttcgt cctcgaccgt cgcggccagg tcgagagcgg |
| | ccagcattag cacgtagtcg |
| 4441 | cggtactggg ttcgcaatcg gtgcacgagc gagcccggca |
| | taccctccga ttcctcgacg |
| 4501 | cactcgacga acgaccgctg cagctggtca tgggacggca |
| | gtgtgacctt gccccgcagc |
| 4561 | aatttccagg actgcggatg ggcgaccagg tgatcgccca |
| | acgccagcga cgagcccagc |
| 4621 | accgagaaca gccgcccgcg cagactgcgt tcgcgcagca |
| | gagccgcgtt gagctcgtcc |
| 4681 | catccggtgt ctggattctc cgacagccgg atcaaggcgc |
| | gcagcgcggc atcggcgtcc |
| 4741 | ggagcgcgtg acagcgacca cagcaggtcg acgtgcgcct |
| | gatcctcgtg ccgatcccac |
| 4801 | cccagctgag ccagacgctc accagcaggg gggtcaacta |
| | atccgagccg gccaacgctg |
| 4861 | ggcaacttcg gccgctgcgt ggcgagtttg gtcacgacca |
| | cgacggtagc gcaaagcgcg |
| 4921 | tcggcgtcgg atcaaccggt agatctgggc tacagcgaca |
| | ggtaggtgcg cagctcgtat |
| 4981 | ggcgtgacgt ggctgcggta gttcgcccac tccgtgcgct |
| | tgttgcgcaa gaaaaagtca |
| 5041 | aaaacgtgct cccccaaggc ctccgcgacg agttcggagg |
| | cctccatggc gcgcagcgca |
| 5101 | ctatccaaac tggacggcaa ttctcggtac cccatcgctc |
| | ggcgttcctc gggtgtgagg |
| 5161 | tcccatacgt tgtcctcggc ctgcgggccc agcacgtaac |
| | ccttctctac accccgcaat |
| 5221 | cccgcggcca gcagcacggc gaatgtcaga tagggattgc |
| | acgccgaatc agggctgcgt |
| 5281 | acttcgaccc gccgcgacga ggtcttgtgc ggcgtgtaca |
| | tcggcacccg cactagggcg |
| 5341 | gatcggttgg cggcccccca cgacgcggcc gtgggcgctt |
| | cgccgccctg caccagccgc |
| 5401 | ttgtaagagt tgacccactg atttgtgacc gcgctgatct |
| | cgcaagcgtg ctccaggatc |
| 5461 | ccggcgatga acgatttacc cacttccgac agctgcagcg |
| | gatcatcagc gctgtggaac |
| 5521 | gcgttgacat caccctcgaa caggctcatg tgggtgtgca |
| | tcgccgagcc cgggtgctgg |
| 5581 | ccgaatggct tgggcatgaa cgacgcccgg gcgccctctt |
| | ccagcgcgac ttctttgatg |
| 5641 | acgtagcgga aggtcatcac gttgtcagcc atcgacagag |
| | cgtcggcaaa ccgcaggtcg |
| 5701 | atctcctgct ggccgggtgc gccttcgtga tggctgaact |
| | ccaccgagat gcccatgaat |
| 5761 | tccagggcat cgatcgcgtg gcggcgaaag ttcaaggcgg |
| | agtcgtgcac cgcttggtcg |
| 5821 | aaatagccgg cgttgtcgac cgggacgggc accgacccgt |
| | cctcgggtcc gggcttgagc |
| 5881 | aggaagaact cgatttcggg atgcacgtag caggagaagc |
| | cgagttcgcc ggccttcgtc |
| 5941 | agctgccgcc gcaacacgtg ccgcgggtcc gcccacgacg |
| | gcgagccgtc cggcatggtg |
| 6001 | atgtcgcaaa acatccgcgc tgagtggtgg tggccggaac |
| | tggtggccca gggcagcacc |
| 6061 | tggaaggtcg acgggtccgg gtgcgccacc gtatcggatt |
| | ccgagacccg cgcaaagccc |
| 6121 | tcgatcgagg atccgtcgaa gccgatgcct tcctcgaagg |
| | cgccctcgag ttcggctggg |
| 6181 | gcgatggcga ccgacttgag gaaaccgagc acgtctgtga |
| | accacagccg gacgaagcgg |
| 6241 | atgtcgcgtt cttccagggt acgaagaacg aattccttct |
| | gtcggtccat acctcgaaca |
| 6301 | gtatgcactg tctgttaaaa ccgtgttacc gatgcccggc |
| | cagaagcgtt gcggggcggc |
| 6361 | ccgcaagggg agtgcgcggt gagttcaggg cgcgcaccgc |
| | agactcgtcg gcggcaaggt |
| 6421 | cccgtcgaga aaatagtgca tcaccgcaga gtccacacac |
| | tggttgccat cgaacaccgc |
| 6481 | agtgtgttgg gtgccgtcga aggtgatcag cggtgcgccc |
| | agctggcggg ccaggtctac |
| 6541 | cccggactga tacggagtgg ccgggtcgtg ggtggtggac |
| | accacgacga ccttgccagc |
| 6601 | cccggccggc gccgcggggt gcggcgtcga cgttgccggc |
| | accggccaca gcgcgcacag |
| 6661 | atcgcggggg gcggatccgg tgaactgccc gtagctaagg |
| | aacggggcga cctgacggat |
| 6721 | ccgttggtcg gcggccaccc aggccgctgg atcggccggt |
| | gtgggcgcat cgacgcaccg |
| 6781 | gaccgcgttg aacgcgtcct ggtcgttgct gtagtgcccg |
| | tctgcatccc ggccgtcata |
| 6841 | gtcgtcggca agcaccagca agtcgccggc gtcgctgccg |
| | cgctgcagcc ccagcagacc |
| 6901 | actggtcagg tacttccagc gctgagggct gtacagcgcg |
| | ttgatggtgc ccgtcgtcgc |
| 6961 | gtcggcgtag ctcaggccac gtggatccga cgtcttaccc |
| | ggcttctgca ccagcgggtc |
| 7021 | aaccagggcg tggtagcggt tgacccactg ggccgagtcg |
| | gtgcccagag ggcaggccgg |
| 7081 | cgagcgggcg cagtcggcgg cgtagtcatt gaaagcggtc |
| | tgaaatcccg ccatttggct |
| 7141 | gatgctttcc tcgattgggc taacggctgg atcgatagcg |
| | ccgtcgagga ccatcgcccg |
| 7201 | cacatgagta ccgaaccgtt ccaggtaagc ggtgcccaac |
| | tcggtgccgt agctgtatcc |
| 7261 | gaggtagttg atctgatcgt cacctaacgc ttggcgaacc |
| | atgtccatgt cccgtgcgac |
| 7321 | ggacgcggta ccgatattgg ccaagaagct gaagcccatc |
| | cggtcaacac agtcctgggc |
| 7381 | caactgccgg tagacctgtt cgacgtgggt gacaccggcc |
| | ggactgtagt cggccatcgg |
| 7441 | atcgcgccgg tacgcgtcga actcggcgtc ggtgcgacac |
| | cgcaacgcag gggtcgagtg |
| 7501 | gccgacccct ctcgggtcga agcccaccag gtcgaagtgg |
| | cggagaatgt cggtgtcggc |
| 7561 | gatcgcgggt gccatagcgg cgaccatgtc gaccgccgac |
| | gccccgggtc ccccaggatt |
| 7621 | gaccagcagt gctccgaatc gctgtcccgt cgcggggacg |
| | cggatcaccg ccaacttcgc |
| 7681 | ttgtgtccca ccgggttggt cgtagtcgac ggggacggac |
| | accgtcgcgc agcgtgcagt |
| 7741 | gcgaatttcg ctggtgtcgg cgatgaactc gcggcagctg |
| | ttccaactct gttgcggcgc |
| 7801 | cacgaccggc gcacccgggg tttggccggc gccgggttct |
| | tcagtcgcgc cggccaacgg |
| 7861 | gggcgctgct aggggcagtc cgccgagcag caacccgaag |
| | gacagcagcg ccgagctcaa |
| 7921 | cggtctgcgg cgccacatgg ccgccatcgt ctcaccggcg |
| | aatacctgtg acggcgcgaa |
| 7981 | atgatcacac cttcgtttct tcgccccgct agcacttggc |
| | gccgctgggc ggcgtggtgc |
| 8041 | cgccgattaa atacgccgtc acgtactcgt caatgcagct |
| | gtcgccctgg aataccaccg |
| 8101 | tgtgctgggt tccgtcgaag gtcagcaacg aaccgcgaag |
| | ctggttcgcc aggtcgaccc |
| 8161 | cggccttgta cggcgtcgcc gggtcatggg tggtggatac |
| | caccaccgtc ggcactaggc |
| 8221 | cgggcgccga gacggcatgg ggctgacttg tgggtggcac |
| | cggccagaac gcgcaggtgc |
| 8281 | ccagcggcgc atcaccggtg aacttcccgt agctcatgaa |
| | cggtgcgatc tcccgggcgc |
| 8341 | ggcggtcttc gtcgatgacc ttgtcgcgat cggtaaccgg |
| | gggctgatcg acgcaattga |
| 8401 | tcgccacccg cgcgtcaccg gaattgttgt agcggccgtg |
| | cgagtcccga cgcatgtaca |
| 8461 | tgtcggccag agccagcagg gtgtctccgc gattgtcgac |
| | cagctccgac agcccgtcgg |
| 8521 | tcaagtgttg ccacagattc ggtgagtaca gcgccataat |
| | ggtgcccacg atggcgtcgc |
| 8581 | tataactcag cccgcgcgga tccttcgtgc gcgccggcct |
| | gctgatcctc gggttgtccg |
| 8641 | ggtcgaccaa cggatcgacc aggctgtggt agacctcgac |
| | ggctttggcc gggtcggcgc |
| 8701 | ccagcgggca gcccgcgttc ttggcgcagt cggcggcata |
| | gttgttgaac gcgtcctgga |
| 8761 | agcccttggc ctggcgcagc tccgcctcga tgggatcggc |
| | attggggtcg acggcaccgt |
| 8821 | cgagaatcat tgcccgcacc cgctgcggaa attcctcggc |
| | atacgcggag ccgatccggg |
| 8881 | tgccgtacga gtagcccagg taggtcagct tgtcgtcgcc |
| | caacgccgcg cgaatggcat |
| 8941 | ccaggtcctt ggcgacgttg accgtcccga catgggccag |
| | aaagttcttg cccatcttgt |
| 9001 | ccacacagcg accgacgaat tgcttggtct cgttctcgat |
| | gtgcgccaca ccctcccggc |
| 9061 | tgtagtcaac ctgcggctcg gcccgcagcc ggtcgttgtc |
| | ggcatcggag ttgcaccaga |
| 9121 | tcgccggccg ggacgacgcc accccgcggg ggtcgaaccc |
| | aaccaggtcg aacctttcgt |
| 9181 | gcacccgctt cggcaatgtc tggaagacgc ccaaggcggc |
| | ctcgataccg gattcgccgg |
| 9241 | gtccaccggg atttatgacc agcgaaccga tcttgtctcc |
| | cgtcgccgga aagcgaatca |
| 9301 | gcgccagcgc cgccacgtca ccatcggggc ggtcgtagtc |
| | gaccggtaca gcgagcttgc |
| 9361 | cgcataacgc gccgccgggg atctttactt gcgggtttga |
| | cgaccggcac ggtgtccact |
| 9421 | ccaccggctg gcccagcttc ggctccgcca tacgagcgcg |
| | tcccccgacc acgcggatgc |
| 9481 | agcccacaag aaccaacgcc acggcggcga gcgcggccca |
| | gatcaacagc atgcgcgcga |
| 9541 | tcttgtcgcg gcgagacagc ctcatgccca caatgctgcc |
| | agagcagacc cgagatcctg |
| 9601 | gccagcggcc accgtcggcc gactaaccgg ccgctgccag |
| | cagtcctgcc atcgccgatg |
| 9661 | gcgaactcgt cggccatccc ccatacgtcc ggtaacagat |
| | ccgggcaaga caccgacccg |
| 9721 | tcgaccggat ccggcacggg cgcgtcggcc tcggcggtgc |
| | acaactgcga catcaggttg |
| 9781 | gcgctggcac cccgtccacg ccggcatggt gcaccttggc |
| | catcgcccga gggcgatccc |
| 9841 | cgatgccgtc caccccttcg acgaacccat ctcccacggc |
| | ggtcgccggc agcgacgcga |
| 9901 | tgtggccgca gatctccgag agttcggccc gcccgcccgg |
| | cgacggcaac ccgatgccgt |
| 9961 | gcaagtgacg atcgatgtga ggttcaaggt tcagcgcact |
| | gctggcaagc tttttccgaa |
| 10021 | accgcggcct cgccttgatc tggagtcaga acgcgtcacg |
| | cagccggtca aaggcgtaac |
| 10081 | ccatgctcga gcaaacatgc atgggctgag tggacgtttc |
| | cagacacagc aactggcgtc |
| 10141 | caggccactg agccgctgca tgcgcgatgg tatgccgatg |
| | ggggccccgg gcgcgtctga |
| 10201 | ggggaagaag tggcagactg tcagggtccg acgaacccgg |
| | ggaccctaac gggccacgag |
| 10261 | gatcgacccg accaccatta gggacagtga tgtctgagca |
| | gactatctat ggggccaata |
| 10321 | cccccggagg ctccgggccg cggaccaaga tccgcaccca |
| | ccacctacag agatggaagg |
| 10381 | ccgacggcca caagtgggcc atgctgacgg cctacgacta |
| | ttcgacggcc cggatcttcg |
| 10441 | acgaggccgg catcccggtg ctgctggtcg gtgattcggc |
| | ggccaacgtc gtgtacggct |
| 10501 | acgacaccac cgtgccgatc tccatcgacg agctgatccc |
| | gctggtccgt ggcgtggtgc |
| 10561 | ggggtgcccc gcacgcactg gtcgtcgccg acctgccgtt |
| | cggcagctac gaggcggggc |
| 10621 | ccaccgccgc gttggccgcc gccacccggt tcctcaagga |
| | cggcggcgca catgcggtca |
| 10681 | agctcgaggg cggtgagcgg gtggccgagc aaatcgcctg |
| | tctgaccgcg gcgggcatcc |
| 10741 | cggtgatggc acacatcggc ttcaccccgc aaagcgtcaa |
| | caccttgggc ggcttccggg |
| 10801 | tgcagggccg cggcgacgcc gccgaacaaa ccatcgccga |
| | cgcgatcgcc gtcgccgaag |
| 10861 | ccggagcgtt tgccgtcgtg atggagatgg tgcccgccga |
| | gttggccacc cagatcaccg |
| 10921 | gcaagcttac cattccgacg gtcgggatcg gcgctgggcc |
| | caactgcgac ggccaggtcc |
| 10981 | tggtatggca ggacatggcc gggttcagcg gcgccaagac |
| | cgcccgcttc gtcaaacggt |
| 11041 | atgccgatgt cggtggtgaa ctacgccgtg ctgcaatgca |
| | atacgcccaa gaggtggccg |
| 11101 | gcggggtatt ccccgctgac gaacacagtt tctgaccaag |
| | ccgaatcagc ccgatgcgcg |
| 11161 | ggcattgcgg tggcgccctg gatgccgtcg acgccggatt |
| | gccggcgcgg acgcgccagc |
| 11221 | gggacccatc ggcgtcgcgt tcgccggttg agcccggggt |
| | gagcccagac attcgatgtg |
| 11281 | cccaacacca tccgccacag cccaattgat gtggcactct |
| | atgcatgcct atccccgacc |
| 11341 | aaccaccacc gcggcgacgc atcatgaccg gaggcgaaga |
| | tgccagtaga ggcgcccaga |
| 11401 | ccagcgcgcc atctggaggt cgagcgcaag ttcgacgtga |
| | tcgagtcgac ggtgtcgccg |
| 11461 | tcgttcgagg gcatcgccgc ggtggttcgc gtcgagcagt |
| | cgccgaccca gcagctcgac |
| 11521 | gcggtgtact tcgacacacc gtcgcacgac ctggcgcgca |
| | accagatcac cttgcggcgc |
| 11581 | cgcaccggcg gcgccgacgc cggctggcat ctgaagctgc |
| | cggccggacc cgacaagcgc |
| 11641 | accgagatgc gagcaccgct gtccgcatca ggcgacgctg |
| | tgccggccga gttgttggat |
| 11701 | gtggtgctgg cgatcgtccg cgaccagccg gttcagccgg |
| | tcgcgcggat cagcactcac |
| 11761 | cgcgaaagcc agatcctgta cggcgccggg ggcgacgcgc |
| | tggcggaatt ctgcaacgac |
| 11821 | gacgtcaccg catggtcggc cggggcattc cacgccgctg |
| | gtgcagcgga caacggccct |
| 11881 | gccgaacagc agtggcgcga atgggaactg gaactggtca |
| | ccacggatgg gaccgccgat |
| 11941 | accaagctac tggaccggct agccaaccgg ctgctcgatg |
| | ccggtgccgc acctgccggc |
| 12001 | cacggctcca aactggcgcg ggtgctcggt gcgacctctc |
| | ccggtgagct gcccaacggc |
| 12061 | ccgcagccgc cggcggatcc agtacaccgc gcggtgtccg |
| | agcaagtcga gcagctgctg |
| 12121 | ctgtgggatc gggccgtgcg ggccgacgcc tatgacgccg |
| | tgcaccagat gcgagtgacg |
| 12181 | acccgcaaga tccgcagctt gctgacggat tcccaggagt |
| | cgtttggcct gaaggaaagt |
| 12241 | gcgtgggtca tcgatgaact gcgtgagctg gccgatgtcc |
| | tgggcgtagc ccgggacgcc |
| 12301 | gaggtactcg gtgaccgcta ccagcgcgaa ctggacgcgc |
| | tggcgccgga gctggtacgc |
| 12361 | ggccgggtgc gcgagcgcct ggtagacggg gcgcggcggc |
| | gataccagac cgggctgcgg |
| 12421 | cgatcactga tcgcattgcg gtcgcagcgg tacttccgtc |
| | tgctcgacgc tctagacgcg |
| 12481 | cttgtgtccg aacgcgccca tgccacttct ggggaggaat |
| | cggcaccggt aaccatcgat |
| 12541 | gcggcctacc ggcgagtccg caaagccgca aaagccgcaa |
| | agaccgccgg cgaccaggcg |
| 12601 | ggcgaccacc accgcgacga ggcattgcac ctgatccgca |
| | agcgcgcgaa gcgattacgc |
| 12661 | tacaccgcgg cggctactgg ggcggacaat gtgtcacaag |
| | aagccaaggt catccagacg |
| 12721 | ttgctaggcg atcatcaaga cagcgtggtc agccgggaac |
| | atctgatcca gcaggccata |
| 12781 | gccgcgaaca ccgccggcga ggacaccttc acctacggtc |
| | tgctctacca acaggaagcc |
| 12841 | gacttggccg agcgctgccg ggagcagctt gaagccgcgc |
| | tgcgcaaact cgacaaggcg |
| 12901 | gtccgcaaag cacgggattg agcccgccag gggcggacga |
| | gttggcctgt aagccggatt |
| 12961 | ctgttccgcg ccgccacagc caagctaacg gcggcacggc |
| | ggcgaccatc catctggaca |
| 13021 | caccgttacc gggtgcctcg agcggcctac ccgcaggctc |
| | gggcgagcaa ccctcaagcg |
| 13081 | cctgcgcggc cgcactttcg gtgcggcctt cttggccttg |
| | cttcgggtgg ggtttgccta |
| 13141 | gccaccccgg tcacccggaa tgctggtgcg ctcttaccgc |
| | accgtttcac ccttgccacc |
| 13201 | acgaggatgg cggtctgttt tctgtggcac tttcccgcga |
| | gtcacctcgg attgccgtta |
| 13261 | gcaatcaccc tgctctgtga agtccggact ttcctcgact |
| | cgacgctgaa cctcgtgaat |
| 13321 | ccacacaagc cctacgcgag ccgcggccgc ccagccaact |
| | catccgcgac gaccacgcta |
| 13381 | ccccgctggg cggtgtcgcg gccagtgtga ccgctggacg |
| | acacggctag tcggacagcc |
| 13441 | gatccggcgg gcagtcctta tcgtggactg gtgacacggt |
| | gggacaaacg cgtcgactcc |
| 13501 | ggcgactggg acgccatcgc tgccgaggtc agcgagtacg |
| | gtggcgcact gctacctcgg |
| 13561 | ctgatcaccc ccggcgaggc cgcccggctg cgcaagctgt |
| | acgccgacga cggcctgttt |
| 13621 | cgctcgacgg tcgatatggc atccaagcgg tacggcgccg |
| | ggcagtatcg atatttccat |
| 13681 | gccccctatc ccgagtgatc gagcgtctca agcaggcgct |
| | gtatcccaaa ctgctgccga |
| 13741 | tagcgcgcaa ctggtgggcc aaactgggcc gggaggcgcc |
| | ctggccagac agccttgatg |
| 13801 | actggttggc gagctgtcat gccgccggcc aaacccgatc |
| | cacagcgctg atgttgaagt |
| 13861 | acggcaccaa cgactggaac gccctacacc aggatctcta |
| | cggcgagttg gtgtttccgc |
| 13921 | tgcaggtggt gatcaacctg agcgatccgg aaaccgacta |
| | caccggcggc gagttcctgc |
| 13981 | ttgtcgaaca gcggcctcgc gcccaatccc ggggtaccgc |
| | aatgcaactt ccgcagggac |
| 14041 | atggttatgt gttcacgacc cgtgatcggc cggtgcggac |
| | tagccgtggc tggtcggcat |
| 14101 | ctccagtgcg ccatgggctt tcgactattc gttccggcga |
| | acgctatgcc atggggctga |
| 14161 | tctttcacga cgcagcctga ttgcacgcca tctatagata |
| | gcctgtctga ttcaccaatc |
| 14221 | gcaccgacga tgccccatcg gcgtagaact cggcgatgct |
| | cagcgatgcc agatcaagat |
| 14281 | gcaaccgata taggacgccc gacccggcat ccaacgccag |
| | ccgcaacaac attttgatcg |
| 14341 | gcgtgacatg tgacaccacc agcaccgtcg cgccttcgta |
| | gccaacgatg atccgatcac |
| 14401 | gtccccgccg aacccgccgc agcacgtcgt cgaagctttc |
| | cccacccggg ggcgtgatgc |
| 14461 | tggtgtcctg cagccagcga cggtgcagct cgggatcgcg |
| | ttctgcggcc tccgcgaacg |
| 14521 | tcagcccctc ccaggcgccg aagtcggtct cgaccaggtc |
| | gtcatcgacg accacgtcca |
| 14581 | gggccagggc tctggcggcg gtcaccgcgg tgtcgtaagc |
| | ccgctgtagc ggcgaggaga |
| 14641 | ccaccgcagc gatcccgccg cgccgcgcca gatacccggc |
| | cgccgcacca acctggcgcc |
| 14701 | accccacctc gttcaacccc gggttgccgc gccccgaata |
| | gcggcgttgc tccgacagct |
| 14761 | ccgtctgccc gtggcgcaac aaaagtagtc gggtgggtgt |
| | accgcgggcg ccggtccagc |
| 14821 | cgggagatgt cggtgactcg gtcgcaacga ttttggcagg |
| | atccgcatcc gccgcagccg |
| 14881 | attgcgcggc ggcgtccatc gcgtcattgg ccaaccggtc |
| | tgcatacgtg ttccgggcac |
| 14941 | gcggaaccca ctcgtagttg atcctgcgaa actgggacgc |
| | caacgcctga gcctggacat |
| 15001 | agagcttcag cagatccggg tgcttgacct tccaccgccc |
| | ggacatctgc tccaccacca |
| 15061 | gcttggagtc catcagcacc gcggcctcgg tggcacctag |
| | tttcacggcg tcgtccaaac |
| 15121 | cggctatcag gccgcggtat tcggcgacgt tgttcgtcgc |
| | ccggccgatc gcctgcttgg |
| 15181 | actcggccag cacggtggag tgatcggcgg tccacaccac |
| | cgcgccgtat ccggccggtc |
| 15241 | cgggattgcc ccgcgatccg ccgtcggctt cgatgacaac |
| | tttcactcct caaatccttc |
| 15301 | gagccgcaac aagatcgctc cgcattccgg gcagcgcacc |
| | acttcatcct cggcggccgc |
| 15361 | cgagatctgg gccagctcgc cgcggccgat ctcgatccgg |
| | caggcaccac atcgatgacc |
| 15421 | ttgcaaccgc ccggcccctg gcccgcctcc ggcccgctgt |
| | ctttcgtaga gccccgcaag |
| 15481 | ctcgggatca agtgtcgccg tcagcatgtc gcgttgcgat |
| | gaatgttggt gccgggcttg |
| 15541 | gtcgatttcg gcaagtgcct cgtccaaagc ctgctgggcg |
| | gcggccaggt cggcccgcaa |
| 15601 | cgcttggagc gcccgcgact cggcggtctg ttgagcctgc |
| | agctcctcgc ggcgttccag |
| 15661 | cacctccagc agggcatctt ccaaactggc ttgacggcgt |
| | tgcaagctgt cgagctcgtg |
| 15721 | ctgcagatca gccaattgct tggcgtccgt tgcacccgaa |
| | gtgagcaacg accggtcccg |
| 15781 | gtcgccacgc ttacgcaccg catcgatctc cgactcaaaa |
| | cgcgacacct ggccgtccaa |
| 15841 | gtcctccgcc gcgattcgca gggccgccat cctgtcgttg |
| | gcggcgttgt gctcggcctg |
| 15901 | cacctgctgg taagccgccc gctgcggcag atgggtagcc |
| | cgatgcgcga tccgggtcag |
| 15961 | ctcagcatcc agcttcgcca attccagtag cgaccgttgc |
| | tgtgccactc cggctttcat |
| 16021 | gcctgatctc tcccagtttc gtgatcgagg ttccacgggt |
| | cggtgcagat ggtgcacaca |
| 16081 | cgcaccggca gcgacgcgcc gaaatgagac cgcaacactt |
| | cggcggcctg gccgcaccac |
| 16141 | gggaattcgc ttgcccaatg cgcgacgtcg atcagggcca |
| | cttgcgaagc tcggcaatgc |
| 16201 | tcgtcggctg gatgatgtcg cagatcggcc gtaacgtacg |
| | cttgcacgtc cgcggcggcc |
| 16261 | acggtggcaa gcaacgagtc cccggcgccg ccgcagaccg |
| | cgacccgcga caccagcagg |
| 16321 | tcgggatccc cggcggcgcg cacaccggtc gcagtcggcg |
| | gcaacgcggc ctccagacgg |
| 16381 | gcaacaaagg tgcgcagcgg ttcgggtttt ggcagtctgc |
| | caatccggcc taacccgctg |
| 16441 | ccgaccggcg gtggtaccag cgcgaagatg tcgaatgccg |
| | gctcctcgta agggtgcgcg |
| 16501 | gcgcgcatcg ccgccaacac ctcggcgcgc gctcgtgcgg |
| | gtgcgacgac ctcgacccgg |
| 16561 | tcctcggcca cccgttcgac ggtaccgacg ctgcctatgg |
| | cgggcgacgc cccgtcgtgc |
| 16621 | gccaggaact gcccggtacc cgcgacactc cagctgcagt |
| | gcgagtagtc gccgatatgg |
| 16681 | ccggcaccgg cctcaaagac cgctgcccgc accgcctctg |
| | agttctcgcg cggcacatag |
| 16741 | atgacccact tgtcgagatc ggccgctccg ggcaccgggt |
| | cgagaacggc gtcgacggtc |
| 16801 | agaccaacag cgtgtgccag cgcgtcggac acacccggcg |
| | acgccgagtc ggcgttggtg |
| 16861 | tgcgcggtaa acaacgagcg accggtccgg atcaggcggt |
| | gcaccagcac accctttggc |
| 16921 | gtgttggccg cgaccgtatc gaccccacgc agtaacaacg |
| | ggtggtgcac caatagcagt |
| 16981 | ccggcctggg gaacctggtc caccaccgcc ggcgtcgcgt |
| | ccaccgcaac ggtcaccgaa |
| 17041 | tccaccacgt cgtcggggtc gccgcacacc agacccaccg |
| | aatcccacga ctgggcaagc |
| 17101 | cgcggcgggt aggcctggtc cagcacgtcg atgacatcgg |
| | ccagccgcac actcatcggc |
| 17161 | gtcctccacg ctttgcccac tcggcgatcg ccgccaccag |
| | cacgggccac tccgggcgca |
| 17221 | ccgccgcccg caggtaccgc gcgtccaggc cgacgaaggt |
| | gtcaccgcgg cgcaccgcaa |
| 17281 | ttcctttgct ctgcaaatag tttcgtaatc cgtcagcatc |
| | ggcgatgttg aacagtacga |
| 17341 | aaggggccgc accatcgacc acctcggcac ccaccgatct |
| | cagtccggcc accatctccg |
| 17401 | cgcgcagcgc cgtcaaccgc accgcatcgg ctgcggcagc |
| | ggcgaccgcc cggggggcgc |
| 17461 | agcaagcagc gatggccgtc agttgcaatg ttcccaacgg |
| | ccagtgcgct cgctgcacgg |
| 17521 | tcaaccgagc cagcacgtct ggcgagccga gcgcgtagcc |
| | cacccgcaat ccggccagcg |
| 17581 | accacgtttt cgtcaagcta cggagcacca gcacatcggg |
| | cagcgagtca tcggccaacg |
| 17641 | attgcggctc gccgggaacc caatcagcga acgcctcgtc |
| | gaccaccagg atgcgtcccg |
| 17701 | gccggcgtaa ctcgagcagc tgctcgcgga ggtgcagcac |
| | cgaggtgggg ttggtcggat |
| 17761 | tacccacgac gacaaggtcg gcgtcgtcag gcacgtgcgc |
| | ggtgtccagc acgaacggcg |
| 17821 | gctttaggac aacatggtgc gccgtgattc cggcagcgct |
| | caaggctatg gccggctcgg |
| 17881 | tgaacgcggg cacgacgatt gctgcccgca ccggacttag |
| | gttgtgcagc aatgcgaatc |
| 17941 | cctccgccgc cccgacgagc gggagcactt cgtcacgggt |
| | tctgccatga cgttcagcga |
| 18001 | ccgcgtcttg cgcccggtgc acatcgtcgg tgctcggata |
| | gcgggccagc tccggcagca |
| 18061 | gcgcggcgag ctgccggacc aaccattccg ggggccggtc |
| | atggcggacg ttgacggcga |
| 18121 | agtccagcac gccgggcgcg acatcctgat caccgtggta |
| | gcgcgccgcg gcaagcgggc |
| 18181 | tagtgtctag actcgccaca gcgtcaaaca gtagtgggcc |
| | ggtgtgcggg ccaagaatcc |
| 18241 | agagcaccgc cgacgcgttg tctacgcggc gacaaccgcg |
| | acatcacagg cagctaacag |
| 18301 | ggcgtcggcg gtgatgatcg tcaggccaag cagctgtgcc |
| | tgggcgatga gcacacggtc |
| 18361 | gaatggatgt cgatggtgat ccggaagctc tgcggtgcgc |
| | agtgtgtgcg tggtcaactg |
| 18421 | acagcggcga cgtgccgcag cggcgcattc gatcgggcac |
| | gtaagaagcc gatggctcgg |
| 18481 | gcggcgggag cttgccgagg cggtagttga tcgcgatctc |
| | ccaggcactg gcggccgaca |
| 18541 | agagaatgct gttgcggacg tcctgaacaa tcgcccgtgt |
| | ttcgttgacg gcatccgcag |
| 18601 | ccaaacgtgg gtgtcgatga ggtagcgctt caccggtgaa |
| | agcgttcgag cacgtcgtct |
| 18661 | gacaacggag cgtccaaatc gtcgggcacg cggtacacgc |
| | catggtcaat gcctaaccgc |
| 18721 | cgagtctcat gaggatgcag cggcacaagc tttgctaccg |
| | gctcgccgcg gcgggcaatc |
| 18781 | tcaacctctg cccgccgtag acgagccgca gcagctcgga |
| | caggcgtgtc ttcgcctcgt |
| 18841 | gaacgccgac ccgcttcgca ggcgcccaga ctttcgcgtc |
| | gaccacctgc tcaccaaact |
| 18901 | tcgcgatcat cgcctgatac cacagcgcca acgggtagcg |
| | gtttgtccaa ccgcttcgtc |
| 18961 | aacgacaatg ggatcgtgac cgacacgacc gcgagcggga |
| | ccaattgccc gcctcctcca |
| 19021 | cgcgccgccg cacggcgcgc atcgtcgccg ggtgaatcgc |
| | cgcagctggt gatcttcgat |
| 19081 | ctggacggca cgctgaccga ctcggcgcgc ggaatcgtat |
| | ccagcttccg acacgcgctc |
| 19141 | aaccacatcg gtgccccagt acccgaaggc gacctggcca |
| | ctcacatcgt cggcccgccc |
| 19201 | atgcatgaga cgctgcgcgc catggggctc ggcgaatccg |
| | ccgaggaggc gatcgtagcc |
| 19261 | taccgggccg actacagcgc ccgcggttgg gcgatgaaca |
| | gcttgttcga cgggatcggg |
| 19321 | ccgctgctgg ccgacctgcg caccgccggt gtccggctgg |
| | ccgtcgccac ctccaaggca |
| 19381 | gagccgaccg cacggcgaat cctgcgccac ttcggaattg |
| | agcagcactt cgaggtcatc |
| 19441 | gcgggcgcga gcaccgatgg ctcgcgaggc agcaaggtcg |
| | acgtgctggc ccacgcgctc |
| 19501 | gcgcagctgc ggccgctacc cgagcggttg gtgatggtcg |
| | gcgaccgcag ccacgacgtc |
| 19561 | gacggggcgg ccgcgcacgg catcgacacg gtggtggtcg |
| | gctggggcta cgggcgcgcc |
| 19621 | gactttatcg acaagacctc caccaccgtc gtgacgcatg |
| | ccgccacgat tgacgagctg |
| 19681 | agggaggcgc taggtgtctg atccgctgca cgtcacattc |
| | gtttgtacgg gcaacatctg |
| 19741 | ccggtcgcca atggccgaga agatgttcgc ccaacagctt |
| | cgccaccgtg gcctgggtga |
| 19801 | cgcggtgcga gtgaccagtg cgggcaccgg gaactggcat |
| | gtaggcagtt gcgccgacga |
| 19861 | gcgggcggcc ggggtgttgc gagcccacgg ctaccctacc |
| | gaccaccggg ccgcacaagt |
| 19921 | cggcaccgaa cacctggcgg cagacctgtt ggtggccttg |
| | gaccgcaacc acgctcggct |
| 19981 | gttgcggcag ctcggcgtcg aagccgcccg ggtacggatg |
| | ctgcggtcat tcgacccacg |
| 20041 | ctcgggaacc catgcgctcg atgtcgagga tccctactat |
| | ggcgatcact ccgacttcga |
| 20101 | ggaggtcttc gccgtcatcg aatccgccct gcccggcctg |
| | cacgactggg tcgacgaacg |
| 20161 | tctcgcgcgg aacggaccga gttgatgccc cgcctagcgt |
| | tcctgctgcg gcccggctgg |
| 20221 | ctggcgttgg ccctggtcgt ggtcgcgttc acctacctgt |
| | gctttacggt gctcgcgccg |
| 20281 | tggcagctgg gcaagaatgc caaaacgtca cgagagaacc |
| | agcagatcag gtattccctc |
| 20341 | gacaccccgc cggttccgct gaaaaccctt ctaccacagc |
| | aggattcgtc ggcgccggac |
| 20401 | gcgcagtggc gccgggtgac ggcaaccgga cagtaccttc |
| | cggacgtgca ggtgctggcc |
| 20461 | cgactgcgcg tggtggaggg ggaccaggcg tttgaggtgt |
| | tggccccatt cgtggtcgac |
| 20521 | ggcggaccaa ccgtcctggt cgaccgtgga tacgtgcggc |
| | cccaggtggg ctcgcacgta |
| 20581 | ccaccgatcc cccgcctgcc ggtgcagacg gtgaccatca |
| | ccgcgcggct gcgtgactcc |
| 20641 | gaaccgagcg tggcgggcaa agacccattc gtcagagacg |
| | gcttccagca ggtgtattcg |
| 20701 | atcaataccg gacaggtcgc cgcgctgacc ggagtccagc |
| | tggctgggtc ctatctgcag |
| 20761 | ttgatcgaag accaacccgg cgggctcggc gtgctcggcg |
| | ttccgcatct agatcccggg |
| 20821 | ccgttcctgt cctatggcat ccaatggatc tcgttcggca |
| | ttctggcacc gatcggcttg |
| 20881 | ggctatttcg cctacgccga gatccgggcg cgccgccggg |
| | aaaaagcggg gtcgccacca |
| 20941 | ccggacaagc caatgacggt cgagcagaaa ctcgctgacc |
| | gctacggccg ccggcggtaa |
| 21001 | accaacatca cggccaatac cgcagccccc gcctggacca |
| | cccgcgacag caccacggcg |
| 21061 | cggcgcagat cggccacctt gggcgaccgg ccgtcgccca |
| | aggtgggccg gatctgcaac |
| 21121 | tcatggtggt accgggtggg cccacccagc cgcacgtcaa |
| | gcgccccagc aaacgccgcc |
| 21181 | tcgacgacac cggcgttggg gctgggatgg cgggcggcgt |
| | cgcgccgcca ggcccgtacc |
| 21241 | gcaccgcggg gcgacccacc gaccaccggc gcgcagatca |
| | ccaccagcac cgccgtcgcc |
| 21301 | cgtgcgccaa catagttggc ccagtcatcc aatcgtgctg |
| | cagcccaacc gaatcggaga |
| 21361 | taacgcggcg agcggtagcc gatcatcgag tccagggtgt |
| | tgatggcacg atatcccagc |
| 21421 | accgcaggca cgccgctcga agccgcccac agcagcggca |
| | ccacctgggc gtcggcggtg |
| 21481 | ttttcggcca ccgactccag cgcggcacgc gtcaggcccg |
| | ggccgcccag ctgggccggg |
| 21541 | tcacgcccgc acagcgacgg cagcagccgt cgcgccgcct |
| | cgacatcgtc gcgctccaac |
| 21601 | aggtccgata tctggcggcc ggtgcgcgcc agcgaagttc |
| | cgcccagcgc tgcccaggtg |
| 21661 | gccgtcgcgg tggccgccac gggccaggac ctgccgggta |
| | gccgctgcag tgccgcgccg |
| 21721 | agcaagccca ccgcgccgac cagcaggccg acgtgtaccg |
| | caccggcgac ccggccgtca |
| 21781 | cggtaggtga tctgctccag cttggcggcc gcccgaccga |
| | acagggccac cggatgacct |
| 21841 | cgtttggggt cgccgaacac gacgtcgagc aggcagccga |
| | tcagcacgcc gacggccctg |
| 21901 | gtctgccagg tcgatgcaaa cactccggca gcgtcgcaca |
| | cgtggtctac gctcagctat |
| 21961 | ttatgacctc atacggcagc tatccacgat gaagcggcca |
| | gctacccggg ttgccgacct |
| 22021 | gttgaacccg gcggcaatgt tgttgccggc agcgaatgtc |
| | atcatgcagc tggcagtgcc |
| 22081 | gggtgtcggg tatggcgtgc tggaaagccc ggtggacagc |
| | ggcaacgtct acaagcatcc |
| 22141 | gttcaagcgg gcccggacca ccggcaccta cctggcggtg |
| | gcgaccatcg ggacggaatc |
| 22201 | cgaccgagcg ctgatccggg gtgccgtgga cgtcgcgcac |
| | cggcaggttc ggtcgacggc |
| 22261 | ctcgagccca gtgtcctata acgccttcga cccgaagttg |
| | cagctgtggg tggcggcgtg |
| 22321 | tctgtaccgc tacttcgtgg accagcacga gtttctgtac |
| | ggcccactcg aagatgccac |
| 22381 | cgccgacgcc gtctaccaag acgccaaacg gttagggacc |
| | acgctgcagg tgccggaggg |
| 22441 | gatgtggccg ccggaccggg tcgcgttcga cgagtactgg |
| | aagcgctcgc ttgatgggct |
| 22501 | gcagatcgac gcgccggtgc gcgagcatct tcgcggggtg |
| | gcctcggtag cgtttctccc |
| 22561 | gtggccgttg cgcgcggtgg ccgggccgtt caacctgttt |
| | gcgacgacgg gattcttggc |
| 22621 | accggagttc cgcgcgatga tgcagctgga gtggtcacag |
| | gcccagcagc gtcgcttcga |
| 22681 | gtggttactt tccgtgctac ggttagccga ccggctgatt |
| | ccgcatcggg cctggatctt |
| 22741 | cgtttaccag ctttacttgt gggacatgcg gtttcgcgcc |
| | cgacacggcc gccgaatcgt |
| 22801 | ctgatagagc ccggccgagt gtgagcctga cagcccgaca |
| | ccggcggcgt gtgtcgcgtc |
| 22861 | gccaggttca cgctcggcga tctagagccg ccgaaaacct |
| | acttctgggt tgcctcccga |
| 22921 | atcaacgtgc tgatctgctc gagcagctca cgcatatcgg |
| | cgcgcatcgc atccaccgcg |
| 22981 | gcatacaggt cggccttggt cgccggcagc tggtccgacg |
| | tcattggccg caccggcggt |
| 23041 | gctgtctgtc gcgccgcgct gtcgctttga aacccaggtc |
| | gctcacccac gaccacgaca |
| 23101 | ctgccatatc cggcgccccg ccgacaacga agcacagcta |
| | gccggtgggc gcggacggga |
| 23161 | tcgaaccgcc gaccgctggt gtgtaaaacc agagctctac |
| | cgctgagcta cgcgcccatg |
| 23221 | accgccgcag gctacacgcc ttgcggccaa gcacccaaaa |
| | ccttaggccg taagcgccgc |
| 23281 | cagagcgtcg gtccacagcc gctgatcgcg aacttcaccc |
| | ggctgcttca tctcggcgaa |
| 23341 | ccgaatgatc cctgaccgat cgaccacaaa ggtgccccgg |
| | ttagcgatgc cggcctgctc |
| 23401 | gttgaagacg ccgtaggcct gactgaccgc gccgtgtggc |
| | cagaagtccg acaacagcgg |
| 23461 | aaacgtgaat ccgctctgcg tcgcccagat cttgtgagtg |
| | ggtggcgggc ccaccgaaat |
| 23521 | cgccagcgcg gcgctgtcgt cgttctcaaa ctcgggcagg |
| | tgatcacgca actggtccag |
| 23581 | ctcgccctgg cagatgcccg tgaacgccaa cggaaagaac |
| | accaacagca cgttctttgc |
| 23641 | accccggtag ccgcgcaggg tgacaagctg ctgattctgg |
| | tcgcgcaacg tgaagtcagg |
| 23701 | ggcggtggct ccgacgttca gcatcagcgc ttgccagccc |
| | gcgatttcgg ctgtaccaat |
| 23761 | ctgctggcgc tccagttgcc cagattgacc gacgaggtcg |
| | gcatcagccc agctgtgggc |
| 23821 | gccgcctcgg caatctcggc gggcaataca tggccgggct |
| | ggccggtctt gggcgtcacc |
| 23881 | acccaaatca caccgtcctc ggcgagcggg ccgatcgcat |
| | ccatcagggt gtccaccaaa |
| 23941 | tcgccgtcgc catcacgcca ccacaacagg acgacatcga |
| | tgacctcgtc ggtgtcttca |
| 24001 | tcgagcaact ctcccccgca cgcttcttcg atggccgcgc |
| | ggatgtcgtc gtcggtgtct |
| 24061 | tcgtcccagc cccattcctg gataagttgg tctcgttgga |
| | tgcccaattt gcgggcgtag |
| 24121 | ttcgaggcgt gatccgccgc gaccaccgtg gaacctcctt |
| | cagtctccgc gggccatgtg |
| 24181 | cacaccgtcg cgatgggcat tatcgtcgca cagccagaac |
| | cggtccaccc gcccgcctca |
| 24241 | gaaggcggcc acgcacattg tcaatgcctt tgtcttggtg |
| | tcgttgagcc gatcaacccg |
| 24301 | ccggttgaat tccgctgtcg acgcgtgcgc accgatggca |
| | tttgccaccg cgcgggccgc |
| 24361 | gtcgacatat gcgttgagcg catcccccag ttgcgcggac |
| | agcgcggcgc tcagactgcc |
| 24421 | tgagaccgtc gaggcactgt tgttgagcgc gtcgatggcc |
| | ggaccttcgg tcggcccggt |
| 24481 | gttgcggccc tgattgaacg cggccacgta ggcgttcacc |
| | ttgtcgatgg cgtccttgct |
| 24541 | ggtggccgcc agcgcgtcac acgaggtgcg aatcgccttg |
| | gtcgtcagcg attgttggcg |
| 24601 | ctgcgactcc cggatgctcg acgtcgccgc cgaagccgac |
| | accgacgcgg acaccgacga |
| 24661 | gcggtaggcc ggtgcgacgt tggtgtcggg catggccgta |
| | ccgtcggtga cagtggtaca |
| 24721 | tccgacgatc cccatcagca gcagcgcgat gcagccgagc |
| | gccagggcgc ctcgcctggg |
| 24781 | gagctccccc ccgtgcctgc gaggcacggc gcgccatccg |
| | atgagcacgg catgtgaggt |
| 24841 | tacctggtcg cagcgcgacc gcgctggccg tggtgtgtcg |
| | cgcatccgca gaaccgagcg |
| 24901 | gagtgcggct atccgccgcc gacgccggtg cggcacgata |
| | gggggacgac catctaaaca |
| 24961 | gcacgcaagc ggaagcccgc cacctacagg agtagtgcgt |
| | tgaccaccga tttcgcccgc |
| 25021 | cacgatctgg cccaaaactc aaacagcgca agcgaacccg |
| | accgagttcg ggtgatccgc |
| 25081 | gagggtgtgg cgtcgtattt gcccgacatt gatcccgagg |
| | agacctcgga gtggctggag |
| 25141 | tcctttgaca cgctgctgca acgctgcggc ccgtcgcggg |
| | cccgctacct gatgttgcgg |
| 25201 | ctgctagagc gggccggcga gcagcgggtg gccatcccgg |
| | cattgacgtc taccgactat |
| 25261 | gtcaacacca tcccgaccga gctggagccg tggttccccg |
| | gcgacgaaga cgtcgaacgt |
| 25321 | cgttatcgag cgtggatcag atggaatgcg gccatcatgg |
| | tgcaccgtgc gcaacgaccg |
| 25381 | ggtgtgggcg tgggtggcca tatctcgacc tacgcgtcgt |
| | ccgcggcgct ctatgaggtc |
| 25441 | ggtttcaacc acttcttccg cggcaagtcg cacccgggcg |
| | gcggcgatca ggtgttcatc |
| 25501 | cagggccacg cttccccggg aatctacgcg cgcgccttcc |
| | tcgaagggcg gttgaccgcc |
| 25561 | gagcaactcg acggattccg ccaggaacac agccatgtcg |
| | gcggcgggtt gccgtcctat |
| 25621 | ccgcacccgc ggctcatgcc cgacttctgg gaattcccca |
| | ccgtgtcgat gggtttgggc |
| 25681 | ccgctcaacg ccatctacca ggcacggttc aaccactatc |
| | tgcatgaccg cggtatcaaa |
| 25741 | gacacctccg atcaacacgt gtggtgtttt ttgggcgacg |
| | gcgagatgga cgaacccgag |
| 25801 | agccgtgggc tggcccacgt cggcgcgctg gaaggcttgg |
| | acaacttgac cttcgtgatc |
| 25861 | aactgcaatc tgcagcgact cgacggcccg gtgcgcggca |
| | acggcaagat catccaggag |
| 25921 | ctggagtcgt tcttccgcgg tgccggctgg aacgtcatca |
| | aggtggtgtg gggccgcgaa |
| 25981 | tgggatgccc tgctgcacgc cgaccgcgac ggtgcgctgg |
| | tgaatttaat gaatacaaca |
| 26041 | cccgatggcg attaccagac ctataaggcc aacgacggcg |
| | gctacgtgcg tgaccacttc |
| 26101 | ttcggccgcg acccacgcac caaggcgctg gtggagaaca |
| | tgagcgacca ggatatctgg |
| 26161 | aacctcaaac ggggcggcca cgattaccgc aaggtttacg |
| | ccgcctaccg cgccgccgtc |
| 26221 | gaccacaagg gacagccgac ggtgatcctg gccaagacca |
| | tcaaaggcta cgcgctgggc |
| 26281 | aagcatttcg aaggacgcaa tgccacccac cagatgaaaa |
| | aactgaccct ggaagacctt |
| 26341 | aaggagtttc gtgacacgca gcggattccg gtcagcgacg |
| | cccagcttga agagaatccg |
| 26401 | tacctgccgc cctactacca ccccggcctc aacgccccgg |
| | agattcgtta catgctcgac |
| 26461 | cggcgccggg ccctcggggg ctttgttccc gagcgcagga |
| | ccaagtccaa agcgctgacc |
| 26521 | ctgccgggtc gcgacatcta cgcgccgctg aaaaagggct |
| | ctgggcacca ggaggtggcc |
| 26581 | accaccatgg cgacggtgcg cacgttcaaa gaagtgttgc |
| | gcgacaagca gatcgggccg |
| 26641 | cggatagtcc cgatcattcc cgacgaggcc cgcaccttcg |
| | ggatggactc ctggttcccg |
| 26701 | tcgctaaaga tctataaccg caatggccag ctgtataccg |
| | cggttgacgc cgacctgatg |
| 26761 | ctggcctaca aggagagcga agtcgggcag atcctgcacg |
| | agggcatcaa cgaagccggg |
| 26821 | tcggtgggct cgttcatcgc ggccggcacc tcgtatgcga |
| | cgcacaacga accgatgatc |
| 26881 | cccatttaca tcttctactc gatgttcggc ttccagcgca |
| | ccggcgatag cttctgggcc |
| 26941 | gcggccgacc agatggctcg agggttcgtg ctcggggcca |
| | ccgccgggcg caccaccctg |
| 27001 | accggtgagg gcctgcaaca cgccgacggt cactcgttgc |
| | tgctggccgc caccaacccg |
| 27061 | gcggtggttg cctacgaccc ggccttcgcc tacgaaatcg |
| | cctacatcgt ggaaagcgga |
| 27121 | ctggccagga tgtgcgggga gaacccggag aacatcttct |
| | tctacatcac cgtctacaac |
| 27181 | gagccgtacg tgcagccgcc ggagccggag aacttcgatc |
| | ccgagggcgt gctgcggggt |
| 27241 | atctaccgct atcacgcggc caccgagcaa cgcaccaaca |
| | aggcgcagat cctggcctcc |
| 27301 | ggggtagcga tgcccgcggc gctgcgggca gcacagatgc |
| | tggccgccga gtgggatgtc |
| 27361 | gccgccgacg tgtggtcggt gaccagttgg ggcgagctaa |
| | accgcgacgg ggtggccatc |
| 27421 | gagaccgaga agctccgcca ccccgatcgg ccggcgggcg |
| | tgccctacgt gacgagagcg |
| 27481 | ctggagaatg ctcggggccc ggtgatcgcg gtgtcggact |
| | ggatgcgcgc ggtccccgag |
| 27541 | cagatccgac cgtgggtgcc gggcacatac ctcacgttgg |
| | gcaccgacgg gttcggcttt |
| 27601 | tccgacactc ggcccgccgc tcgccgctac ttcaacaccg |
| | acgccgaatc ccaggtggtc |
| 27661 | gcggttttgg aggcgttggc gggcgacggc gagatcgacc |
| | catcggtgcc ggtcgcggcc |
| 27721 | gcccgccagt accggatcga cgacgtggcg gctgcgcccg |
| | agcagaccac ggatcccggt |
| 27781 | cccggggcct aacgccggcg agccgaccgc ctttggccga |
| | atcttccaga aatctggcgt |
| 27841 | agcttttagg agtgaacgac aatcagttgg ctccagttgc |
| | ccgcccgagg tcgccgctcg |
| 27901 | aactgctgga cactgtgccc gattcgctgc tgcggcggtt |
| | gaagcagtac tcgggccggc |
| 27961 | tggccaccga ggcagtttcg gccatgcaag aacggttgcc |
| | gttcttcgcc gacctagaag |
| 28021 | cgtcccagcg cgccagcgtg gcgctggtgg tgcagacggc |
| | cgtggtcaac ttcgtcgaat |
| 28081 | ggatgcacga cccgcacagt gacgtcggct ataccgcgca |
| | ggcattcgag ctggtgcccc |
| 28141 | aggatctgac gcgacggatc gcgctgcgcc agaccgtgga |
| | catggtgcgg gtcaccatgg |
| 28201 | agttcttcga agaagtcgtg cccctgctcg cccgttccga |
| | agagcagttg accgccctca |
| 28261 | cggtgggcat tttgaaatac agccgcgacc tggcattcac |
| | cgccgccacg gcctacgccg |
| 28321 | atgcggccga ggcacgaggc acctgggaca gccggatgga |
| | ggccagcgtg gtggacgcgg |
| 28381 | tggtacgcgg cgacaccggt cccgagctgc tgtcccgggc |
| | ggccgcgctg aattgggaca |
| 28441 | ccaccgcgcc ggcgaccgta ctggtgggaa ctccggcgcc |
| | cggtccaaat ggctccaaca |
| 28501 | gcgacggcga cagcgagcgg gccagccagg atgtccgcga |
| | caccgcggct cgccacggcc |
| 28561 | gcgctgcgct gaccgacgtg cacggcacct ggctggtggc |
| | gatcgtctcc ggccagctgt |
| 28621 | cgccaaccga gaagttcctc aaagacctgc tggcagcatt |
| | cgccgacgcc ccggtggtca |
| 28681 | tcggccccac ggcgcccatg ctgaccgcgg cgcaccgcag |
| | cgctagcgag gcgatctccg |
| 28741 | ggatgaacgc cgtcgccggc tggcgcggag cgccgcggcc |
| | cgtgctggct agggaacttt |
| 28801 | tgcccgaacg cgccctgatg ggcgacgcct cggcgatcgt |
| | ggccctgcat accgacgtga |
| 28861 | tgcggcccct agccgatgcc ggaccgacgc tcatcgagac |
| | gctagacgca tatctggatt |
| 28921 | gtggcggcgc gattgaagct tgtgccagaa agttgttcgt |
| | tcatccaaac acagtgcggt |
| 28981 | accggctcaa gcggatcacc gacttcaccg ggcgcgatcc |
| | cacccagcca cgcgatgcct |
| 29041 | atgtccttcg ggtggcggcc accgtgggtc aactcaacta |
| | tccgacgccg cactgaagca |
| 29101 | tcgacagcaa tgccgtgtca tagattccct cgccggtcag |
| | agggggtcca gcaggggccc |
| 29161 | cggaaagata ccaggggcgc cgtcggacgg aaagtgatcc |
| | agacaacagg tcgcgggacg |
| 29221 | atctcaaaaa catagcttac aggcccgttt tgttggttat |
| | atacaaaaac ctaagacgag |
| 29281 | gttcataatc tgttacaccg cgcaaaaccg tcttcacagt |
| | gttctcttag acacgtgatt |
| 29341 | gcgttgctcg cacccggaca gggttcgcaa accgagggaa |
| | tgttgtcgcc gtggcttcag |
| 29401 | ctgcccggcg cagcggacca gatcgcggcg tggtcgaaag |
| | ccgctgatct agatcttgcc |
| 29461 | cggctgggca ccaccgcctc gaccgaggag atcaccgaca |
| | ccgcggtcgc ccagccattg |
| 29521 | atcgtcgccg cgactctgct ggcccaccag gaactggcgc |
| | gccgatgcgt gctcgccggc |
| 29581 | aaggacgtca tcgtggccgg ccactccgtc ggcgaaatcg |
| | cggcctacgc aatcgccggt |
| 29641 | gtgatagccg ccgacgacgc cgtcgcgctg gccgccaccc |
| | gcggcgccga gatggccaag |
| 29701 | gcctgcgcca ccgagccgac cggcatgtct gcggtgctcg |
| | gcggcgacga gaccgaggtg |
| 29761 | ctgagtcgcc tcgagcagct cgacttggtc ccggcaaacc |
| | gcaacgccgc cggccagatc |
| 29821 | gtcgctgccg gccggctgac cgcgttggag aagctcgccg |
| | aagacccgcc ggccaaggcg |
| 29881 | cgggtgcgtg cactgggtgt cgccggagcg ttccacaccg |
| | agttcatggc gcccgcactt |
| 29941 | gacggctttg cggcggccgc ggccaacatc gcaaccgccg |
| | accccaccgc cacgctgctg |
| 30001 | tccaaccgcg acgggaagcc ggtgacatcc gcggccgcgg |
| | cgatggacac cctggtctcc |
| 30061 | cagctcaccc aaccggtgcg atgggacctg tgcaccgcga |
| | cgctgcgcga acacacagtc |
| 30121 | acggcgatcg tggagttccc ccccgcgggc acgcttagcg |
| | gtatcgccaa acgcgaactt |
| 30181 | cggggggttc cggcacgcgc cgtcaagtca cccgcagacc |
| | tggacgagct ggcaaaccta |
| 30241 | taaccgcgga ctcggccaga acaaccacat acccgtcagt |
| | tcgatttgta cacaacatat |
| 30301 | tacgaaggga agcatgctgt gcctgtcact caggaagaaa |
| | tcattgccgg tatcgccgag |
| 30361 | atcatcgaag aggtaaccgg tatcgagccg tccgagatca |
| | ccccggagaa gtcgttcgtc |
| 30421 | gacgacctgg acatcgactc gctgtcgatg gtcgagatcg |
| | ccgtgcagac cgaggacaag |
| 30481 | tacggcgtca agatccccga cgaggacctc gccggtctgc |
| | gtaccgtcgg tgacgttgtc |
| 30541 | gcctacatcc agaagctcga ggaagaaaac ccggaggcgg |
| | ctcaggcgtt gcgcgcgaag |
| 30601 | attgagtcgg agaaccccga tgccgttgcc aacgttcagg |
| | cgaggcttga ggccgagtcc |
| 30661 | aagtgagtca gccttccacc gctaatggcg gtttccccag |
| | cgttgtggtg accgccgtca |
| 30721 | cagcgacgac gtcgatctcg ccggacatcg agagcacgtg |
| | gaagggtctg ttggccggcg |
| 30781 | agagcggcat ccacgcactc gaagacgagt tcgtcaccaa |
| | gtgggatcta gcggtcaaga |
| 30841 | tcggcggtca cctcaaggat ccggtcgaca gccacatggg |
| | ccgactcgac atgcgacgca |
| 30901 | tgtcgtacgt ccagcggatg ggcaagttgc tgggcggaca |
| | gctatgggag tccgccggca |
| 30961 | gcccggaggt cgatccagac cggttcgccg ttgttgtcgg |
| | caccggtcta ggtggagccg |
| 31021 | agaggattgt cgagagctac gacctgatga atgcgggcgg |
| | cccccggaag gtgtccccgc |
| 31081 | tggccgttca gatgatcatg cccaacggtg ccgcggcggt |
| | gatcggtctg cagcttgggg |
| 31141 | cccgcgccgg ggtgatgacc ccggtgtcgg cctgttcgtc |
| | gggctcggaa gcgatcgccc |
| 31201 | acgcgtggcg tcagatcgtg atgggcgacg ccgacgtcgc |
| | cgtctgcggc ggtgtcgaag |
| 31261 | gacccatcga ggcgctgccc atcgcggcgt tctccatgat |
| | gcgggccatg tcgacccgca |
| 31321 | acgacgagcc tgagcgggcc tcccggccgt tcgacaagga |
| | ccgcgacggc tttgtgttcg |
| 31381 | gcgaggccgg tgcgctgatg ctcatcgaga cggaggagca |
| | cgccaaagcc cgtggcgcca |
| 31441 | agccgttggc ccgattgctg ggtgccggta tcacctcgga |
| | cgcctttcat atggtggcgc |
| 31501 | ccgcggccga tggtgttcgt gccggtaggg cgatgactcg |
| | ctcgctggag ctggccgggt |
| 31561 | tgtcgccggc ggacatcgac cacgtcaacg cgcacggcac |
| | ggcgacgcct atcggcgacg |
| 31621 | ccgcggaggc caacgccatc cgcgtcgccg gttgtgatca |
| | ggccgcggtg tacgcgccga |
| 31681 | agtctgcgct gggccactcg atcggcgcgg tcggtgcgct |
| | cgagtcggtg ctcacggtgc |
| 31741 | tgacgctgcg cgacggcgtc atcccgccga ccctgaacta |
| | cgagacaccc gatcccgaga |
| 31801 | tcgaccttga cgtcgtcgcc ggcgaaccgc gctatggcga |
| | ttaccgctac gcagtcaaca |
| 31861 | actcgttcgg gttcggcggc cacaatgtgg cgcttgcctt |
| | cgggcgttac tgaagcacga |
| 31921 | catcgcgggt cgcgaggccc gaggtggggg tccccccgct |
| | tgcgggggcg agtcggaccg |
| 31981 | atatggaagg aacgttcgca agaccaatga cggagctggt |
| | taccgggaaa gcctttccct |
| 32041 | acgtagtcgt caccggcatc gccatgacga ccgcgctcgc |
| | gaccgacgcg gagactacgt |
| 32101 | ggaagttgtt gctggaccgc caaagcggga tccgtacgct |
| | cgatgaccca ttcgtcgagg |
| 32161 | agttcgacct gccagttcgc atcggcggac atctgcttga |
| | ggaattcgac caccagctga |
| 32221 | cgcggatcga actgcgccgg atgggatacc tgcagcggat |
| | gtccaccgtg ctgagccggc |
| 32281 | gcctgtggga aaatgccggc tcacccgagg tggacaccaa |
| | tcgattgatg gtgtccatcg |
| 32341 | gcaccggcct gggttcggcc gaggaactgg tcttcagtta |
| | cgacgatatg cgcgctcgcg |
| 32401 | gaatgaaggc ggtctcgccg ctgaccgtgc agaagtacat |
| | gcccaacggg gccgccgcgg |
| 32461 | cggtcgggtt ggaacggcac gccaaggccg gggtgatgac |
| | gccggtatcg gcgtgcgcat |
| 32521 | ccggcgccga ggccatcgcc cgtgcgtggc agcagattgt |
| | gctgggagag gccgatgccg |
| 32581 | ccatctgcgg cggcgtggag accaggatcg aagcggtgcc |
| | catcgccggg ttcgctcaga |
| 32641 | tgcgcatcgt gatgtccacc aacaacgacg accccgccgg |
| | tgcatgccgc ccattcgaca |
| 32701 | gggaccgcga cggctttgtg ttcggcgagg gcggcgccct |
| | tctgttgatc gagaccgagg |
| 32761 | agcacgccaa ggcacgtggc gccaacatcc tggcccggat |
| | catgggcgcc agcatcacct |
| 32821 | ccgatggctt ccacatggtg gccccggacc ccaacgggga |
| | acgcgccggg catgcgatta |
| 32881 | cgcgggcgat tcagctggcg ggcctcgccc ccggcgacat |
| | cgaccacgtc aatgcgcacg |
| 32941 | ccaccggcac ccaggtcggc gacctggccg aaggcagggc |
| | catcaacaac gccttgggcg |
| 33001 | gcaaccgacc ggcggtgtac gcccccaagt ctgccctcgg |
| | ccactcggtg ggcgcggtcg |
| 33061 | gcgcggtcga atcgatcttg acggtgctcg cgttgcgcga |
| | tcaggtgatc ccgccgacac |
| 33121 | tgaatctggt aaacctcgat cccgagatcg atttggacgt |
| | ggtggcgggt gaaccgcgac |
| 33181 | cgggcaatta ccggtatgcg atcaataact cgttcggatt |
| | cggcggccac aacgtggcaa |
| 33241 | tcgccttcgg acggtactaa accccagcgt tacgcgacag |
| | gagacctgcg atgacaatca |
| 33301 | tggcccccga ggcggttggc gagtcgctcg acccccgcga |
| | tccgctgttg cggctgagca |
| 33361 | acttcttcga cgacggcagc gtggaattgc tgcacgagcg |
| | tgaccgctcc ggagtgctgg |
| 33421 | ccgcggcggg caccgtcaac ggtgtgcgca ccatcgcgtt |
| | ctgcaccgac ggcaccgtga |
| 33481 | tgggcggcgc catgggcgtc gaggggtgca cgcacatcgt |
| | caacgcctac gacactgcca |
| 33541 | tcgaagacca gagtcccatc gtgggcatct ggcattcggg |
| | tggtgcccgg ctggctgaag |
| 33601 | gtgtgcgggc gctgcacgcg gtaggccagg tgttcgaagc |
| | catgatccgc gcgtccggct |
| 33661 | acatcccgca gatctcggtg gtcgtcggtt tcgccgccgg |
| | cggcgccgcc tacggaccgg |
| 33721 | cgttgaccga cgtcgtcgtc atggcgccgg aaagccgggt |
| | gttcgtcacc gggcccgacg |
| 33781 | tggtgcgcag cgtcaccggc gaggacgtcg acatggcctc |
| | gctcggtggg ccggagaccc |
| 33841 | accacaagaa gtccggggtg tgccacatcg tcgccgacga |
| | cgaactcgat gcctacgacc |
| 33901 | gtgggcgccg gttggtcgga ttgttctgcc agcaggggca |
| | tttcgatcgc agcaaggccg |
| 33961 | aggccggtga caccgacatc cacgcgctgc tgccggaatc |
| | ctcgcgacgt gcctacgacg |
| 34021 | tgcgtccgat cgtgacggcg atcctcgatg cggacacacc |
| | gttcgacgag ttccaggcca |
| 34081 | attgggcgcc gtcgatggtg gtcgggctgg gtcggctgtc |
| | gggtcgcacg gtgggtgtac |
| 34141 | tggccaacaa cccgctacgc ctgggcggct gcctgaactc |
| | cgaaagcgca gagaaggcag |
| 34201 | cgcgtttcgt gcggctgtgc gacgcgttcg ggattccgct |
| | ggtggtggtg gtcgatgtgc |
| 34261 | cgggctatct gcccggtgtc gaccaggagt ggggtggcgt |
| | ggtgcgccgt ggcgccaagt |
| 34321 | tgctgcacgc gttcggcgag tgcaccgttc cgcgggtcac |
| | gctggtcacc cgaaagacct |
| 34381 | acggcggggc atacattgcg atgaactccc ggtcgttgaa |
| | cgcgaccaag gtgttcgcct |
| 34441 | ggccggacgc cgaggtcgcg gtgatgggcg ctaaggcggc |
| | cgtcggcatc ctgcacaaga |
| 34501 | agaagttggc cgccgctccg gagcacgaac gcgaagcgct |
| | gcacgaccag ttggccgccg |
| 34561 | agcatgagcg catcgccggc ggggtcgaca gtgcgctgga |
| | catcggtgtg gtcgacgaga |
| 34621 | agatcgaccc ggcgcatact cgcagcaagc tcaccgaggc |
| | gctggcgcag gctccggcac |
| 34681 | ggcgcggccg ccacaagaac atcccgctgt agttctgacc |
| | gcgagcagac gcagaatcgc |
| 34741 | acgcgcgagg tccgcgccgt gcgattctgc gtctgctcgc |
| | cagttatccc cagcggtggc |
| 34801 | tggtcaacgc gaggcgctcc tcgcatgctc ggacggtgcc |
| | taccgacgcg ctaacaattc |
| 34861 | tcgagaaggc cggcgggttc gccaccaccg cgcaattgct |
| | cacggtcatg acccgccaac |
| 34921 | agctcgacgt ccaagtgaaa aacggcggcc tcgttcgcgt |
| | ttggtacggg gtctacgcgg |
| 34981 | cacaagagcc ggacctgttg ggccgcttgg cggctctcga |
| | tgtgttcatg ggggggcacg |
| 35041 | ccgtcgcgtg tctgggcacc gccgccgcgt tgtatggatt |
| | cgacacggaa aacaccgtcg |
| 35101 | ctatccatat gctcgatccc ggagtaagga tgcggcccac |
| | ggtcggtctg atggtccacc |
| 35161 | aacgcgtcgg tgcccggctc caacgggtgt caggtcgtct |
| | cgcgaccgcg cccgcatgga |
| 35221 | ctgccgtgga ggtcgcacga cagttgcgcc gcccgcgggc |
| | gctggccacc ctcgacgccg |
| 35281 | cactacggtc aatgcgctgc gctcgcagtg aaattgaaaa |
| | cgccgttgct gagcagcgag |
| 35341 | gccgccgagg catcgtcgcg gcgcgcgaac tcttaccctt |
| | cgccgacgga cgcgcggaat |
| 35401 | cggccatgga gagcgaggct cggctcgtca tgatcgacca |
| | cgggctgccg ttgcccgaac |
| 35461 | ttcaataccc gatacacggc cacggtggtg aaatgtggcg |
| | agtcgacttc gcctggcccg |
| 35521 | acatgcgtct cgcggccgaa tacgaaagca tcgagtggca |
| | cgcgggaccg gcggagatgc |
| 35581 | tgcgcgacaa gacacgctgg gccaagctcc aagagctcgg |
| | gtggacgatt gtcccgattg |
| 35641 | tcgtcgacga tgtcagacgc gaacccggcc gcctggcggc |
| | ccgcatcgcc cgccacctcg |
| 35701 | accgcgcgcg tatggccggc tgaccgctgg tgagcagacg |
| | cagagtcgca ctgcggccgg |
| 35761 | cgcagtgcga ctctgcgtct gctcgcgctc aacggctgag |
| | gaactcctta gccacggcga |
| 35821 | ctacgcgctc gcgatcccgt ggcaccagac cgatccgggt |
| | ccggcggtcg aggatatcgt |
| 35881 | ccacatccag cgccccctca tgggtcaccg cgtattcgaa |
| | ctccgcccgg gtcacgtcga |
| 35941 | tgccgtcggc gaccggctcg gtgggccgct cacatgtggc |
| | ggcggcagcg acgttggccg |
| 36001 | cctcggcccc gtaccgcgcc accagcgact cgggcaatcc |
| | ggcgcccgat ccgggggccg |
| 36061 | gcccagggtt cgccggtgcg ccgatcagcg gcaggttgcg |
| | agtgcggcac ttcgcggctc |
| 36121 | gcaggtgtcg cagcgtgatg gcgcgattca gcacatcctc |
| | tgccatgtag cggtattccg |
| 36181 | tcagcttgcc gccgaccaca ctgatcacgc ccgacggcga |
| | ttcaaaaaca gcgtggtcac |
| 36241 | gcgaaacgtc ggcggtgcgg ccctggacac cagcaccgcc |
| | ggtgtcgatt agcggccgca |
| 36301 | atcccgcata ggcaccgatg acatccttgg tgccgaccgc |
| | cgtccccaat gcggtgttca |
| 36361 | ccgtatccag caggaacgtg atctcttccg aagacggttg |
| | tggcacatcg ggaatcgggc |
| 36421 | cgggtgcgtc ttcgtcggtc agcccgagat agatccggcc |
| | cagctgctcg ggcatggcga |
| 36481 | acacgaagcg gttcagctca ccggggatcg gaatggtcag |
| | cgcggcagtc ggattggcaa |
| 36541 | acgacttcgc gtcgaagacc agatgtgtgc cgcggctggg |
| | gcgtagcctc agggacgggt |
| 36601 | cgatctcacc cgcccacacg cccgccgcgt tgatgacggc |
| | acgcgccgac agcgcgaacg |
| 36661 | actgccgggt gcgccggtcg gtcaactcca ccgaagtgcc |
| | ggtgacattc gacgcgccca |
| 36721 | cgtaagtgag gatgcgggcg ccgtgctggg ccgcggtgcg |
| | cgcgacggcc atgaccagcc |
| 36781 | gggcgtcgtc gatcaattgc ccgtcgtacg cgagcagacc |
| | accgtcgagg ccgtcccgcc |
| 36841 | gaacggtggg agcaatctcc accacccgtg acgccgggat |
| | tcggcgcgat cggggcaacg |
| 36901 | tcgccgccgg cgtacccgct agcacccgca aagcgtcgcc |
| | ggccaggaaa ccggcacgca |
| 36961 | ccaacgcccg cttggtgtga cccatcgacg gcaacaacgg |
| | gaccagttgc ggcatggcat |
| 37021 | gcacgagatg aggagcgttg cgtgtcatca ggattccgcg |
| | ttcgacggcg ctgcgccggg |
| 37081 | cgatgcccac gttgccgctg gccagatagc gcagaccgcc |
| | gtgcaccaac ttcgagctcc |
| 37141 | agcggctggt gccgaacgcc agatcatgct tttccaccaa |
| | ggccaccgtc agaccgcggg |
| 37201 | tggcagcatc taaggcaatg ccaacaccgg taatgccgcc |
| | gcctatcacg atgacgtcga |
| 37261 | gtgcgccacc gtcggccagt gcggtcaggt cggcggagcg |
| | acgcgccgcg ttgagtgcag |
| 37321 | ccgagtgggg catcagcaca aatatccgtt cagtgcgtgg |
| | gtaagttcgg tggccagcgc |
| 37381 | ggcggaatcg aggatcgaat cgacgatgtc cgcggactgg |
| | atggtcgact gggcgatcag |
| 37441 | caacaccatg gtcgccagtc gacgagcgtc gccggagcgc |
| | acactgcccg accgctgcgc |
| 37501 | cactgtcagc cgggcggcca acccctcgat caggacctgc |
| | tggctggtgc cgaggcgctc |
| 37561 | ggtgatgtac accctggcca gctccgagtg catgaccgac |
| | atgatcagat cgtcaccccg |
| 37621 | caaccggtcg gccaccgcga caatctgctt taccaacgct |
| | tcccggtcgt ccccgtcgag |
| 37681 | gggcacctcc cgcagcacgt cggcgatatg gctggtcagc |
| | atggacgcca tgatcgaccg |
| 37741 | ggtgtccggc cagcgacggt atacggtcgg gcggctcacg |
| | cccgcgcgcc gggcgatctc |
| 37801 | ggcaagtgtc acccggtcca cgccgtaatc gacgacgcag |
| | ctcgccgctg cccgcaggat |
| 37861 | acgaccaccg gtatccgcgc ggtcattact cattgacagc |
| | atgtgtaata ctgtaacgcg |
| 37921 | tgactcaccg cgaggaactc cttccaccga tgaaatggga |
| | cgcgtgggga gatcccgccg |
| 37981 | cggccaagcc actttctgat ggcgtccggt cgttgctgaa |
| | gcaggttgtg ggcctagcgg |
| 38041 | actcggagca gcccgaactc gaccccgcgc aggtgcagct |
| | gcgcccgtcc gccctgtcgg |
| 38101 | gggcagacca |

### 5.9. X-linked Inhibitor of Apoptosis Protein (“XIAP”)

GenBank Accession # U45880:

(SEQ ID NO: 25)

| | |
|---|---|
| 1 | gaaaaggtgg acaagtccta ttttcaagag aagatgactt |
| | ttaacagttt tgaaggatct |
| 61 | aaaacttgtg tacctgcaga catcaataag gaagaagaat |
| | ttgtagaaga gtttaataga |
| 121 | ttaaaaactt ttgctaattt tccaagtggt agtcctgttt |
| | cagcatcaac actggcacga |
| 181 | gcagggtttc tttatactgg tgaaggagat accgtgcggt |
| | gctttagttg tcatgcagct |
| 241 | gtagatagat ggcaatatgg agactcagca gttggaagac |
| | acaggaaagt atccccaaat |
| 301 | tgcagattta tcaacggctt ttatcttgaa aatagtgcca |
| | cgcagtctac aaattctggt |
| 361 | atccagaatg gtcagtacaa agttgaaaac tatctgggaa |
| | gcagagatca ttttgcctta |
| 421 | gacaggccat ctgagacaca tgcagactat cttttgagaa |
| | ctgggcaggt tgtagatata |
| 481 | tcagacacca tatacccgag gaaccctgcc atgtattgtg |
| | aagaagctag attaaagtcc |
| 541 | tttcagaact ggccagacta tgctcaccta accccaagag |
| | agttagcaag tgctggactc |
| 601 | tactacacag gtattggtga ccaagtgcag tgcttttgtt |
| | gtggtggaaa actgaaaaat |
| 661 | tgggaacctt gtgatcgtgc ctggtcagaa cacaggcgac |
| | actttcctaa ttgcttcttt |
| 721 | gttttgggcc ggaatcttaa tattcgaagt gaatctgatg |
| | ctgtgagttc tgataggaat |
| 781 | ttcccaaatt caacaaatct tccaagaaat ccatccatgg |
| | cagattatga agcacggatc |
| 841 | tttacttttg ggacatggat atactcagtt aacaaggagc |
| | agcttgcaag agctggattt |
| 901 | tatgctttag gtgaaggtga taaagtaaag tgctttcact |
| | gtggaggagg gctaactgat |
| 961 | tggaagccca gtgaagaccc ttgggaacaa catgctaaat |
| | ggtatccagg gtgcaaatat |
| 1021 | ctgttagaac agaagggaca agaatatata aacaatattc |
| | atttaactca ttcacttgag |
| 1081 | gagtgtctgg taagaactac tgagaaaaca ccatcactaa |
| | ctagaagaat tgatgatacc |
| 1141 | atcttccaaa atcctatggt acaagaagct atacgaatgg |
| | ggttcagttt caaggacatt |
| 1201 | aagaaaataa tggaggaaaa aattcagata tctgggagca |
| | actataaatc acttgaggtt |
| 1261 | ctggttgcag atctagtgaa tgctcagaaa gacagtatgc |
| | aagatgagtc aagtcagact |
| 1321 | tcattacaga aagagattag tactgaagag cagctaaggc |
| | gcctgcaaga ggagaagctt |
| 1381 | tgcaaaatct gtatggatag aaatattgct atcgtttttg |
| | ttccttgtgg acatctagtc |
| 1441 | acttgtaaac aatgtgctga agcagttgac aagtgtccca |
| | tgtgctacac agtcattact |
| 1501 | ttcaagcaaa aaatttttat gtcttaatct aactctatag |
| | taggcatgtt atgttgttct |
| 1561 | tattaccctg attgaatgtg tgatgtgaac tgactttaag |
| | taatcaggat tgaattccat |
| 1621 | tagcatttgc taccaagtag gaaaaaaaat gtacatggca |
| | gtgttttagt tggcaatata |
| 1681 | atctttgaat ttcttgattt ttcagggtat tagctgtatt |
| | atccattttt tttactgtta |
| 1741 | tttaattgaa accatagact aagaataaga agcatcatac |
| | tataactgaa cacaatgtgt |
| 1801 | attcatagta tactgattta atttctaagt gtaagtgaat |
| | taatcatctg gattttttat |
| 1861 | tcttttcaga taggcttaac aaatggagct ttctgtatat |
| | aaatgtggag attagagtta |
| 1921 | atctccccaa tcacataatt tgttttgtgt gaaaaaggaa |
| | taaattgttc catgctggtg |
| 1981 | gaaagataga gattgttttt agaggttggt tgttgtgttt |
| | taggattctg tccattttct |
| 2041 | tgtaaaggga taaacacgga cgtgtgcgaa atatgtttgt |
| | aaagtgattt gccattgttg |
| 2101 | aaagcgtatt taatgataga atactatcga gccaacatgt |
| | actgacatgg aaagatgtca |
| 2161 | gagatatgtt aagtgtaaaa tgcaagtggc gggacactat |
| | gtatagtctg agccagatca |
| 2221 | aagtatgtat gttgttaata tgcatagaac gagagatttg |
| | gaaagatata caccaaactg |
| 2281 | ttaaatgtgg tttctcttcg gggagggggg gattggggga |
| | ggggccccag aggggtttta |
| 2341 | gaggggcctt ttcactttcg acttttttca ttttgttctg |
| | ttcggatttt ttataagtat |
| 2401 | gtagaccccg aagggtttta tgggaactaa catcagtaac |
| | ctaacccccg tgactatcct |
| 2461 | gtgctcttcc tagggagctg tgttgtttcc cacccaccac |
| | ccttccctct gaacaaatgc |
| 2521 | ctgagtgctg gggcactttg |

General Target Region:

Internal Ribosome Entry Site (IRES) in 5′ untranslated region:

(SEQ ID NO: 26)

5′AGCUCCUAUAACAAAAGUCUGUUGCUUGUGUUUCACAUUUUGGAUUU
CCUAAUAUAAUGUUCUCUUUUUAGAAAAGGUGGACAAGUCCUAUUUUC
AAGAGAAG3′

Initial Specific Target Motif:

RNP core binding site within XIAP IRES

| | |
|---|---|
| 5′GGAUUUCCUAAUAUAAUGUUCUCUUUUU3′ | (SEQ ID NO: 27) |

### 5.10. Survivin

GenBank Accession # NM_{—}001168:

(SEQ ID NO: 28)

| | |
|---|---|
| 1 | ccgccagatt tgaatcgcgg gacccgttgg cagaggtggc |
| | ggcggcggca tgggtgcccc |
| 61 | gacgttgccc cctgcctggc agccctttct caaggaccac |
| | cgcatctcta cattcaagaa |
| 121 | ctggcccttc ttggagggct gcgcctgcac cccggagcgg |
| | atggccgagg ctggcttcat |
| 181 | ccactgcccc actgagaacg agccagactt ggcccagtgt |
| | ttcttctgct tcaaggagct |
| 241 | ggaaggctgg gagccagatg acgaccccat agaggaacat |
| | aaaaagcatt cgtccggttg |
| 301 | cgctttcctt tctgtcaaga agcagtttga agaattaacc |
| | cttggtgaat ttttgaaact |
| 361 | ggacagagaa agagccaaga acaaaattgc aaaggaaacc |
| | aacaataaga agaaagaatt |
| 421 | tgaggaaact gcgaagaaag tgcgccgtgc catcgagcag |
| | ctggctgcca tggattgagg |
| 481 | cctctggccg gagctgcctg gtcccagagt ggctgcacca |
| | cttccagggt ttattccctg |
| 541 | gtgccaccag ccttcctgtg ggccccttag caatgtctta |
| | ggaaaggaga tcaacatttt |
| 601 | caaattagat gtttcaactg tgctcctgtt ttgtcttgaa |
| | agtggcacca gaggtgcttc |
| 661 | tgcctgtgca gcgggtgctg ctggtaacag tggctgcttc |
| | tctctctctc tctctttttt |
| 721 | gggggctcat ttttgctgtt ttgattcccg ggcttaccag |
| | gtgagaagtg agggaggaag |
| 781 | aaggcagtgt cccttttgct agagctgaca gctttgttcg |
| | cgtgggcaga gccttccaca |
| 841 | gtgaatgtgt ctggacctca tgttgttgag gctgtcacag |
| | tcctgagtgt ggacttggca |
| 901 | ggtgcctgtt gaatctgagc tgcaggttcc ttatctgtca |
| | cacctgtgcc tcctcagagg |
| 961 | acagtttttt tgttgttgtg tttttttgtt tttttttttt |
| | ggtagatgca tgacttgtgt |
| 1021 | gtgatgagag aatggagaca gagtccctgg ctcctctact |
| | gtttaacaac atggctttct |
| 1081 | tattttgttt gaattgttaa ttcacagaat agcacaaact |
| | acaattaaaa ctaagcacaa |
| 1141 | agccattcta agtcattggg gaaacggggt gaacttcagg |
| | tggatgagga gacagaatag |
| 1201 | agtgatagga agcgtctggc agatactcct tttgccactg |
| | ctgtgtgatt agacaggccc |
| 1261 | agtgagccgc ggggcacatg ctggccgctc ctccctcaga |
| | aaaaggcagt ggcctaaatc |
| 1321 | ctttttaaat gacttggctc gatgctgtgg gggactggct |
| | gggctgctgc aggccgtgtg |
| 1381 | tctgtcagcc caaccttcac atctgtcacg ttctccacac |
| | gggggagaga cgcagtccgc |
| 1441 | ccaggtcccc gctttctttg gaggcagcag ctcccgcagg |
| | gctgaagtct ggcgtaagat |
| 1501 | gatggatttg attcgccctc ctccctgtca tagagctgca |
| | gggtggattg ttacagcttc |
| 1561 | gctggaaacc tctggaggtc atctcggctg ttcctgagaa |
| | ataaaaagcc tgtcatttc |

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

1. A method for identifying a test compound that binds to a target RNA molecule, comprising the steps of:
(a) contacting a detectably labeled target RNA molecule with a library of solid support-attached test compounds under conditions that permit direct binding of the labeled target RNA to a member of the library of solid support-attached test compounds so that a detectably labeled target RNA:support-attached test compound complex is formed;
(b) separating the detectably labeled target RNA:support-attached test compound complex formed in step (a) from uncomplexed target RNA molecules and test compounds; and
(c) determining a structure of the test compound of the RNA support-attached test compound complex.

2. The method of claim 1 in which the target RNA molecule contains an [symbol] TAR element, internal ribosome entry site, “slippery site”, instability element, or [symbol] ylate uridylate-rich element.

3. The method of claim 1 in which the RNA molecule is an element [symbol] ed from the mRNA for tumor necrosis factor alpha (“TNF-α), granulocyte- [symbol] ophage colony stimulating factor (“GM-CSF”), interleukin 2 (“IL-2”), interleukin 6 [symbol] -6”), vascular endothelial growth factor (“VEGF”), human immunodeficiency virus I [symbol] V-1”), hepatitis C virus (“HCV”—genotypes 1a & 1b), ribonuclease P RNA [symbol] aseP”), X-linked inhibitor of apoptosis protein (“XIAP”), or survivin.

4. The method of claim 1 in which the detectably labeled RNA is [symbol] ed with a fluorescent dye, phosphorescent dye, ultraviolet dye, infrared dye, visible radiolabel, enzyme, spectroscopic colorimetric label, affinity tag, or nanoparticle.

5. The method of claim 1 in which the test compound is selected from a [symbol] binatorial library of solid support-attached test compounds comprising peptoids; [symbol] om bio-oligomers; diversomers such as hydantoins, benzodiazepines and dipeptides; [symbol] gous polypeptides; nonpeptidal peptidominetics; oligocarbamates; peptidyl [symbol] honates; peptide nucleic acid libraries; antibody libraries; carbohydrate libraries; and [symbol] organic molecule libraries.

6. The method of claim 5 in which the small organic molecule libraries [symbol] braries of benzodiazepines, isoprenoids, thiazolidinones, metathiazanones, [symbol] lidines, morpholino compounds, or diazepindiones.

7. The method of claim 1 in which screening a library of scid support- [symbol] ed test compounds comprises contacting the test compound with the target nucleic [symbol] n the presence of an aqueous solution wherein the aqueous solution comprises a buffer [symbol] combination of salts.

8. The method of claim 7 wherein the aqueous solution approximates or [symbol] cs physiologic conditions.

9. The method of claim 7 in which the aqueous solution optionally [symbol] er comprises non-specific nucleic acids comprising DNA, yeast tRNA, salmon sperm [symbol] , homoribopolymers, and nonspecific RNAs.

10. The method of claim 7 in which the aqueous solution further [symbol] rises a buffer, a combination of salts, and optionally, a detergent or a surfactant.

11. The method of claim 10 in which the aqueous solution further [symbol] rises a combination of salts, from about 0 mM to about 100 mM KCl, from about 0 [symbol] o about 1 M NaCl, and from about 0 mM to about 200 mM MgCl₂.

12. The method of claim 11 wherein the combination of salts is about [symbol] nM KCl, 500 mM NaCl, and 10 mM MgCl₂.

13. The method of claim 10 wherein the solution optionally comprises [symbol] about 0.01% to about 0.5% (w/v) of a detergent or a surfactant.

14. The method of claim 1 in which separating the detectably labeled [symbol] RNA:support-attached test compound complex formed in step (a) from uncomplexed [symbol] RNA and test compounds is by flow cytometry, affinity chromatography, manual [symbol] mode separation, suspension of beads in electric fields, or microwave.

15. The method of claim 1 in which the library of solid support-attached [symbol] mpounds are small organic molecule libraries.

16. The method of claim 15 in which the structure of the test compound [symbol] rmined by mass spectroscopy, NMR, or vibration spectroscopy.

17. The method of claim 1 in which the library of solid support-attached [symbol] mpounds are peptide or peptide-based libraries.

18. The method of claim 17 in which the structure of the test compound [symbol] rmined by Edman degradation.
